(19) United States
(12) Patent Application Publication
(10) Pub. No.: US 2024/0226168 A1
(43) Pub. Date: Jul. 11, 2024

(54) ENGINEERED NK CELLS AND USES THEREOF

(71) Applicant: UNIVERSITY OF CENTRAL FLORIDA RESEARCH FOUNDATION, INC., Orlando, FL (US)

(72) Inventors: Alicja J. COPIK, Orlando, FL (US); MD Faqrul Hasan, Orlando, FL (US); Tayler CROOM-PEREZ, Orlando, FL (US)

(21) Appl. No.: 18/621,864

(22) Filed: Mar. 29, 2024

Related U.S. Application Data
(63) Continuation of application No. PCT/US2022/077244, filed on Sep. 29, 2022.
(60) Provisional application No. 63/249,801, filed on Sep. 29, 2021.

(51) Int. Cl. A61K 35/17; A61K 39/00; A61K 45/06; A61P 35/00; C07K 16/28; C12N 5/0783

(52) U.S. Cl. CPC A61K 35/17; A61K 45/06; A61P 35/00; C07K 16/2818; C07K 16/2827; C12N 5/0646; A61K 2039/505

(57) ABSTRACT

Disclosed herein are engineered NK cells suppressed in the expression of T Cell Immunoreceptor with Ig And ITIM Domains (TIGIT) and uses thereof for treating cancers and infectious diseases.

### CROSS-REFERENCE TO RELATED APPLICATIONS

This application is a continuation application of International Application No. PCT/US2022/077244, filed Sep. 29, 2022, which claims the benefit of U.S. Provisional Application No. 63/249,801, filed Sep. 29, 2021, which are expressly incorporated herein by reference in their entireties.

### REFERENCE TO SEQUENCE LISTING

The sequence listing submitted on Mar. 29, 2024, as an .XML file entitled “10613-085US1_ST26” created on Mar. 29, 2024, and having a file size of 15,702 bytes is hereby incorporated by reference pursuant to 37 C.F.R. § 1.52(e)(5).

### BACKGROUND

NK cells have gained acceptance as a promising cellular therapy against cancers, in part, because of their robust cytotoxicity, better safety, and their potential as off-the-shelf therapy. NK cells are first responders as part of the innate immune system and have an inherent ability to recognize and directly kill cancerous cells. Unlike T cells, NK cells kill cancer cells in an MHC and antigen independent manner. NK cells express a set of germline-encoded activation receptors and inhibitory receptors that bind with their ligands and the balance of these inhibitory and activating receptors' signaling controls NK cell activity. While activation receptors activate NK cells upon binding with their ligands which are commonly expressed in malignant and virally infected cells, inhibitory receptors prevent NK cell functions upon binding with their ligands usually expressed on healthy cells, such as MHC molecules. NK cell activation and killing of target cells depends on fine-tuning of these activation and inhibitory signals. Importantly, NK cells express FcγR CD16 activating receptors which bind with the Fc region of antibodies targeting tumor antigens and can kill cancer cells in an antibody-dependent cellular cytotoxicity (ADCC) manner. Cancer cells upregulate ligands for inhibitory receptors and binding of these ligands with inhibitory receptors prevents the anti-tumor activity of NK cells and promotes their exhaustion. In various cancers, upregulation of inhibitory receptor expression correlates with exhaustive phenotypes of NK cells including lower cytotoxicity against cancer cells and reduced expression of proinflammatory cytokine IFNγ.

NK cells not only directly kill tumor cells, but also recruit, coordinate and activate other immune cells including cells of the adaptive immune system through secretion of pro-inflammatory cytokines and chemokines. Recent studies have shown NK cells play an important role in the efficacy of many immunotherapies. Furthermore, NK cells can be efficiently expanded, viably cryopreserved without loss of efficacy and are not associated with graft vs. host disease (GVHD), supporting potential safe use of cryopreserved, donor derived material as an off-the-shelf cell therapy. There are now large number of clinical trials assessing efficacy of NK cells, including CAR-NK cells, in various cancer settings. Checkpoint inhibitors against PD-1/PD-L1 and CTLA-4 have shown success against several cancers. However, only a minority of patients can benefit from these treatments. Furthermore, cancers often develop resistance against these therapies and some patients experience immune-related adverse effects. These observations prompted interest in new inhibitory checkpoints to extend the benefits of immunotherapy to more patients and with a better safety profile.

T cell immunoreceptor with immunoglobulin and ITIM domain (TIGIT) is a major inhibitory receptor of both NK and T cells. TIGIT competes with inhibitory receptors CD96 (TACTILE) and PVRIG and activating receptor CD226 (DNAM-1) to bind with their ligands CD155 (PVR) and CD112 (Nectin-2 or PVRL2) which are commonly expressed on cancer cells and antigen-presenting cells (APC) as well as binds PVRL4 (Nectin4), a novel unique ligand of TIGIT that is almost exclusively expressed on tumor cells. TIGIT signaling regulates the tumor immunity cycle in multiple steps. It prevents T cell proliferation, cytotoxicity and the production of proinflammatory cytokine IFNγ by these cells. Previous studies reported that TIGIT is upregulated on both T and NK cells in cancers and often correlates with their exhaustion. In mouse models, blockade of TIGIT restored NK cell cytotoxicity, increased IFNγ and TNFα expression, and promoted tumor-specific T cell immunity. Additionally, TIGIT and PD-1 combined blockade had a synergistic effect improving tumor control and survival in preclinical mouse models and early clinical trials. In a B16 mouse model, the therapeutic efficacy of PD-1 and TIGIT blockade depended on the presence of NK cells, as NK cell depletion abolished the effect. Several anti-TIGIT antibodies alone or in combination with other checkpoint inhibitors (specifically anti-PD-1) are being tested in phase I, II, and III clinical trials. Although phase II trials showed enhancement of progression-free survival (PFS) and overall survival (OS) in NSCLC with this combination, the Phase III SKYSCRAPER-01 did not meet its co-primary endpoint of PFS, while the study was not far enough along to assess OS. Most of the current understanding of TIGIT regulation of immune cell function has been based on T cells and/or comes from murine models; while only limited mechanistic in vitro studies of TIGIT in human NK cells exists and predominantly rely on NK cell lines. There is a lack of understanding of the effect of the TIGIT blockade on the anti-tumor function of human NK cells on either the functional and/or molecular level that is needed when addressing potential shortcomings of the therapy and to evaluate the potential of combination treatments of adoptive NK cells and anti-TIGIT antibodies.

### SUMMARY

In some aspects, disclosed herein is an engineered NK cell that is suppressed in the expression of T cell immunoreceptor with Ig and ITIM domains (TIGIT). The expression of TIGIT can be suppressed by a deletion of a TIGIT gene or a fragment thereof. In some embodiments, the expression of TIGIT is suppressed using a method comprising introducing into the NK cell a CRISPR/Cas endonuclease (Cas)9 system with a CRISPR/Cas guide RNA, wherein the guide RNA targets the TIGIT gene or a fragment thereof. In some aspects, the expression of TIGIT is suppressed by a siRNA or a shRNA that targets a TIGIT polynucleotide. In some aspect, disclosed herein are NK cells of any preceding aspect is a primary NK cell or a NK cell line. Also disclosed herein are NK cells of any preceding aspect wherein the NK cell is an expanded NK cell or a non-expanded NK cell. In some aspects, the NK cell can be exposed in vitro/in vivo to an NK cell expanding composition (e.g., a feeder cell, an engineered PM particle, or an exosome). In some aspects, the feeder cell or engineered particle of any preceding aspect comprises an Fc domain bound to an external surface thereof. In some embodiments, the NK cell expanding composition further comprises an NK cell effector agent (including, for example, IL-21 and/or 41BBL).

The engineered NK cells disclosed herein shows enhanced function against cancer or infectious diseases. Accordingly, disclosed herein are methods of treating, decreasing, inhibiting, reducing, ameliorating, and/or preventing a cancer, metastasis, or an infectious disease in a subject comprising administering to the subject a therapeutically effective amount of the engineered NK cells of any preceding aspect. Similarly, the present disclosure provides therapeutically effective amounts of the engineered NK cells of any preceding aspect for use in treating, decreasing, inhibiting, reducing, ameliorating, and/or preventing a cancer, metastasis, or an infectious disease in a subject. In some aspects, the engineered NK cell can be incubated with a TIGIT inhibitor prior to administering the engineered NK cell to the subject. In some aspects, the method further comprises administering to the subject a therapeutically effective amount of a TIGIT inhibitor and/or a therapeutically effective amount of a checkpoint blockade (e.g., a PD-1 inhibitor, a PD-L1 inhibitor, a PD-L2 inhibitor, or a CTLA-4 inhibitor).

NK cell exhaustion occurs in subjects having cancers or some infectious diseases. The engineered NK cells disclosed herein show enhanced function (including, for example, enhanced cytotoxicity function and/or increased expression of IFNγ, TNFα, and/or CD107a). Accordingly, disclosed herein are methods for reactivating an NK cell, reversing NK cell exhaustion, and/or enhancing NK cell function, wherein said method comprises suppressing the expression of TIGIT of the NK cell or incubating the NK cell with a TIGIT inhibitor.

Also disclosed herein are methods for reactivating an NK cell, reversing NK cell exhaustion, and/or enhancing NK cell function of any preceding aspect and/or methods of treating, decreasing, inhibiting, reducing, ameliorating, and/or preventing a cancer and/or metastasis of any preceding aspect, wherein the cancer is selected from the group consisting of a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In some aspects, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, or eye.

It should be further understood that any of the therapeutic methods described herein are also considered to be medical uses of any of the compositions disclosed herein, for treating any of the cancers or infectious diseases as disclosed herein.

### DESCRIPTION OF DRAWINGS

FIGS. 1A-1H show that blockade of TIGIT signaling enhances the anti-tumor activity of PM21 NK cells. FigurelA shows RNA expression analysis of inhibitory receptors in naïve NK cells and PM21 NK cells by qRT-PCR. Data shown as the mean±SD of 4 biological replicates. FIG. 1B shows frequency of TIGIT expression on naïve NK cells and PM21 NK cells (n=7 per group). FIG. 1C shows representative figure of NK cell cytotoxicity against A549 spheroids in presence of Isotype or anti-TIGIT antibodies. Data shown as the mean of 3 technical replicates. FIGS. 1D-1F show frequency of cells expressing CD107a (FIG. 1D), IFNγ (FIG. 1E), and TNFα (FIG. 1F) in NK cells. Data shows as mean (n=4 per group). After co-culture of PM21 NK cells with A549 spheroids for 7 days, NK cells were restimulated with PVR+K562 cells for 6 hours in presence of Brefeldin A and Golgi stop and were stained with respective antibodies and isotype. All groups except unstimulated PM21 NK cells were stimulated with PVR+K562 cells. FIG. 1G shows representative TIGIT histogram overlap of parental PM21 NK cells (regular NK cells) and TIGIT knockout PM21 NK cells (KO PM21 NK cells) 5 days after TIGIT knockout in NK cells. FIG. 1H shows representative graph of relative expansion of A549 spheroids. Data shows as the mean of 3 technical replicates. For all graphs, P value * <0.05, ** <0.01, *** <0.001.

FIG. 2 shows that PM21-NK cells significantly upregulate TIGIT.

FIG. 3 shows that TIGIT is upregulated upon activation.

FIG. 4 shows that TIGIT is expressed on activated cells with higher cytotoxic function.

FIG. 5 shows that PVR expression enhances NK cell cytotoxicity likely through DNAM-1.

FIG. 6 shows that anti-TIGIT improves killing of 3D A549 cells by PM21 NK cells.

FIG. 7 shows that TIGIT blockade improves PM21-NK cell killing of 3D A549 cells.

FIG. 8 shows that TIGIT KO NK cells have higher overall cytotoxicity.

FIG. 9 shows that TIGIT knockout NK cells can prevent fratricide and restore NK cell numbers.

FIG. 10 shows the effect of Tiragolumab on NK cell fratricide.

FIG. 11 shows that PM21 NK cells significantly upregulate inhibitory receptors.

FIG. 12 shows a schematic showing an experiment design for testing the effect of TIGIT blockade on anti-tumor activity of PM21 NK cells.

FIGS. 13A and 13B show gene set enrichment analysis with all RNA-seq genes.

FIG. 14 shows gene set enrichment analysis with differentially expressed genes.

FIG. 15 illustrates a mechanism by which antibody blockade of TIGIT induces PVR-mediated exhaustion of NK cells during long-term exposure and restores antitumor activity of NK cells against lung cancer cell line spheroids.

FIGS. 16A-16C show that TIGIT⁺ NK cells have increased expression of NK cell receptors compared to TIGIT⁻ NK cells. NK cells were expanded with PM21 particles from T cell depleted PBMCs for 12 days from four donors. Expression of NK cell activating and inhibitory receptors were determined by flow cytometry and gated on TIGIT⁻ or TIGIT⁺ NK cells. Multiple activating and inhibitory receptors on TIGIT⁺ NK cells (red) compared to TIGIT⁻ NK cells (black) were upregulated (FIG. 16A). Percent of NK cells expressing activating receptors CD16, NKp30, NKp46, DNAM1, and NKG2D (FIG. 16B) and inhibitory receptors CD96, TIM3, NKG2A, LAG3, and PD1 (FIG. 16C) were determined for TIGIT⁺ NK cells (red triangles) and TIGIT⁻ NK cells (black circles). Data are presented as a radar plot or scatter plots with donor-pair lines. Statistical significance was determined by multiple paired t-tests. P values are shown as * if p<0.05 or ** if p<0.01.

FIGS. 17A-17D show that TIGIT blockade enhanced PM21-NK cell cytotoxicity against 3D lung tumor spheroids. NK cells were expanded with PM21-particles from T cell-depleted PBMCs obtained from multiple donors for 14-16 days. Cytotoxicity against A549-NLR lung cancer cells in a monolayer, measured by kinetic live-cell imaging, was not significantly improved in the presence of α-TIGIT antibody compared to isotype control. Representative cytotoxicity over time curves from one donor are shown in the presence of 0.3:1 NK cells:A549 cells either with isotype control (black circles) or in presence of α-TIGIT (red triangles) (FIG. 17A). Summary cytotoxicity at 24, 48, and 72 h from multiple donors at 0.3:1 of NK cells are shown (N=3) (FIG. 17A). Dose-dependent cytotoxicity curves are shown for one donor at multiple NK cells: A549 cells ratios at 24, 48, and 72 h (FIG. 17B). Expanded NK cells were co-cultured with A549 tumor spheroids for 7 days. NK cell cytotoxicity was determined by kinetic live-cell imaging. Representative cytotoxicity curves from one donor are shown with isotype control (black circles) or α-TIGIT (red triangles) present (FIG. 17C). Summary NK cell cytotoxicity from multiple donors at 72 h at 1:1 NK:A549 ratio shows TIGIT blockade significantly increased cytotoxicity (N=6 donors) and a relative cytotoxicity increased for each donor when normalized isotype control (FIG. 17C). After 7 days of co-culture, NK cells were analyzed to determine inhibitory and activating receptors expression by flow cytometry. TIGIT blockade did not significantly change the expression of any major inhibitory or activating receptors when co-cultured with A549 spheroids in the presence of α-TIGIT (red triangles) compared to isotype control (black circles) and expression levels were similar to unexposed NK cells (gray squares) (N=3-5 donors) (FIG. 17D). Data are presented as scatter plots with donor-pair lines or as mean with error bars representing standard deviation. Statistical significances were determined by multiple paired t-tests. For dose-dependent cytotoxicity curves, Area Under the Curve (AUC) was determined and the compared by unpaired t-tests. P values are shown as ns if p>0.05, **** if p<0.0001.

FIGS. 18A-18B show that TIGIT blockade enhanced NK cell-mediated killing in multiple long-term 3D lung tumor spheroid models. NK cells were expanded with PM21-particles from T cell-depleted PBMCs obtained from multiple donors for 14-16 days. Expanded NK cells were co-cultured with NCI-H1299-NLR, NCI-H358-NLR or NCI-1975-NLR lung tumor spheroids for 7 days. NK cell cytotoxicity was determined by kinetic live-cell imaging. Representative cytotoxicity curves from one donor are shown for each cell line tested either with isotype control (black circles) or α-TIGIT (red triangles) present (FIG. 18A). Summary NK cell cytotoxicity from multiple donors at 72 h shows TIGIT blockade significantly increases cytotoxicity and enhanced relative cytotoxicity when each donor normalized to isotype control (E:T ratio used were 3:1 for NCI-H1299, 1:1 for NCI-H358 and 1:3 for NCI-H1975; N=6 donors) (FIG. 18B). Data are presented as scatter plots with donor-pair lines or as mean with error bars representing standard deviation. Statistical significances were determined by multiple paired t-tests. P values are shown as * if p<0.05, ** if p<0.01.

FIGS. 19A-19C show that TIGIT blockade prevents PVR-mediated NK cell exhaustion during long-term exposure. NK cells were expanded from T cell depleted PBMCs for 14-16 days. Expanded NK cells were co-cultured with A549 spheroids for 7 days in the presence of α-TIGIT or isotype control. After 7 days of co-culture, NK cells were stimulated with K562 cancer cells with or without PVR expression for 4-6 hours in the presence of Brefeldin A and Golgi Stop and NK cell expression of surface CD107a, IFNγ and TNFα was analyzed with flow cytometry. Unexposed and either unstimulated or stimulated PM21-NK cells were used as controls. Additionally, NK cells were selected after co-culture with A549 spheroids and used for RNA extraction and transcriptomic analysis. A schematic of the experiment is shown in (FIG. 19A). For each IFNγ, TNFα, and CD107a expression, unstimulated unexposed NK cells (gray squares), unexposed stimulated NK cells (black squares), NK cells that were tumor exposed in the presence of isotype (gray circles) or anti-TIGIT antibody (red triangles) are shown for either re-stimulation with PVR⁻K562 cells or PVR+K562 cells. TIGIT blockade preserved IFNγ, TNFα, and CD107a expression when re-challenged with PVR+K562 cells (N=4 donors) (FIG. 19B). GSEA analysis of RNA-seq data shows TIGIT blockade upregulated IFNγ, TNFα, and other related inflammation response gene sets. Summary graphs show upregulated gene sets upon TIGIT blockade based on their −log₁₀(FDR) (FIG. 19C). Data are presented as scatter plots or bar graphs with error bars representing standard deviation. Statistical significance was determined by multiple unpaired t-tests. P values are shown as * if p<0.05, ** if p<0.01, *** if p<0.001, **** if p<0.0001.

FIG. 20 shows that feeder cell-free expansion technology was developed that utilizes plasma membrane particles derived from K562-mbIL21-41BBL cells (PM21-particles) to stimulate NK cell proliferation. This method results in an average 1,700-fold expansion of NK cells, termed PM21-NK cells, in 2 weeks (N=113 from 18 donors).

FIGS. 21A-21B show representative images from live-cell imaging cytotoxicity assay of NLR-expressing A549 cancer cell spheroids incubated with 10,000 NK cells in the presence of α-TIGIT or isotype control after 0, 48, 96, and 144 h show increased NK cell cytotoxicity in the presence of anti-TIGIT antibody (FIG. 21A). Representative raw data from one donor is shown for A549 relative expansion alone (gray squares) or the presence of 0.3:1 NK cells:A549 cells with isotype control (black circles) or anti-TIGIT (red triangles).

FIGS. 22A-22B show that NK cells were expanded with PM21-particles from T cell-depleted PBMCs obtained from multiple donors. These expanded NK cells were co-cultured with K562 cancer cells with or without PVR expression for 4-6 hours in the presence of α-TIGIT or isotype control with Brefeldin A and Golgi Stop. Expression of IFNγ and TNFα and surface CD107a was analyzed by flow cytometry. Compared to NK cells alone (black circles) TIGIT blockade (red triangles) did not significantly change the percentage of donor-matched NK cells expressing IFNγ, TNFα, or surface CD107a upon stimulation with PVR⁻K562 cells or PVR+K562 cells, except a small significant increase in TNFα expressing NK cells upon TIGIT blockade (36%±3% vs. 32%±2% p=0.008) (N=3 donors) (FIG. 22A). The effect of short-term TIGIT blockade on PM21-NK cell cytotoxicity was also determined. PM21-NK cells were co-cultured with PVR⁻or PVR+K562 cells in the presence of anti-TIGIT or isotype control antibodies for 1 hour and NK cell cytotoxicity was determined by annexin V staining of the K562 cells. Concentration-dependent cytotoxicity curves were generated using multiple NK:K562 ratios and the area under the curve determined. Short-term TIGIT blockade did not significantly improve PM21-NK cell cytotoxicity against either PVR⁻or PVR+K562 cells compared to isotype control antibodies (335±6%·ratio vs 325±10%·ratio against PVR⁻K562 cells and 346±3%·ratio vs 341±10%·ratio against PVR+K562 cells) (FIG. 22B).

FIG. 23 shows representative gating strategy and flow cytometry dot plots for raw data used in the analysis of the in vitro exhaustion model.

FIG. 24 shows a schematic summarizing certain findings about TIGIT-knockout NK cells.

FIGS. 25A-25D show that TIGIT can be efficiently knocked out in ex vivo expanded NK cells.

FIGS. 26A-26H show that TIGIT knockout does not change NK cell phenotype.

FIGS. 27A-27I show that TIGIT KO NK cell are more cytotoxic against various tumor spheroids.

FIGS. 28A-28C show that TIGIT KO NK cells kill better via ADCC.

FIGS. 29A-29C show that TIGIT have increased CD107a surface expression, a marker for degranulation after 48 h exposure to A549 spheroids.

FIGS. 30A-30D show a comparison of glycolysis and mitochondrial stress between WT PM21-NK cells and TIGIT KO PM21-NK cells.

FIGS. 31A-31B show that TIGIT KO NK prevents Tiragolumab induced fratricide and results in enhanced cytotoxicity against A549 spheroids in the presence of Tiragolumab.

FIG. 32 shows that TIGIT KO NK cells have improved in vivo persistence.

FIG. 33A-33C show heatmap of representative up and downregulated gene sets upon TIGIT KO in NK cells.

FIGS. 34A-34D show metabolic analysis of TIGIT KO NK cells.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another. In some embodiments, the compositions disclosed herein are administered parenterally, intravenously, intraperitoneally, or subcutaneously, or through arterial infusion, venous infusion, or artificial catheter mediated infusion.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “linker” refers at least a bivalent moiety with a site of attachment for a polypeptide and a site of attachment for another polypeptide. For example, a polypeptide can be attached to the linker at its N-terminus, its C-terminus or via a functional group on one of the side chains. The linker is sufficient to separate the two polypeptides by at least one atom and in some embodiments by more than one atom.

As used herein, the terms “optional” or “optionally” mean that the subsequently described event or circumstance may or may not occur, and that the description includes instances where said event or circumstance occurs and instances where it does not.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “peptide,” “polypeptide” and “protein” are used interchangeably to refer to a polymer of amino acid residues.

The term “polynucleotide” refers to a single or double stranded polymer composed of nucleotide monomers.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as inhibitory effect on NK cells.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 80%, optionally 50% or 25, 10%, 5% or 1%.

As used herein, “N-terminal side” or “amino terminal end” refers to directionality of a peptide, polypeptide, or protein and may not mean the N-terminus. In some aspects, where a chimeric or fusion peptide, polypeptide, or protein is discussed, the N-terminal side may refer only to a component of the chimeric or fusion peptide, polypeptide, or protein and not the entire structure. For example, where a Fc domain is discussed, and the Fc domain is described as fused with its amino terminal end or N-terminal side facing intracellularly, contemplated herein are chimeric or fusion peptides, polypeptides, or proteins wherein the signal anchor is at the N-terminus of the chimeric or fusion construct and actually spans the cellular membrane. Thus, in such a chimera, the trans-membrane anchor is attached to the amino terminal side of the Fc domain, with the directionality of the Fc domain has the N-terminal side facing the cell which is inverted relative to an Fc domain on a typical B cell which would typically have the carboxy end spanning the cellular membrane and amino terminal end extending to the extracellular matrix.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

### Engineered NK Cells

In some aspects, disclosed herein is an engineered NK cell that is suppressed in the expression of T Cell Immunoreceptor With Ig And ITIM Domains (TIGIT). The expression of TIGIT can be suppressed using any means, including, for example, by a deletion of a TIGIT gene or a fragment thereof (e.g., one or more exons of a TIGIT gene), or by a siRNA or a shRNA that targets a TIGIT polynucleotide.

“TIGIT” refers herein to a polypeptide that, in humans, is encoded by the TIGIT gene. In some embodiments, the TIGIT polypeptide is that identified in one or more publicly available databases as follows: HGNC: 26838, NCBI Entrez Gene: 201633, Ensembl: ENSG00000181847, OMIM®: 612859, UniProtKB/Swiss-Prot: Q495A1. In some embodiments, the TIGIT polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The TIGIT polypeptide of SEQ ID NO: 1 may represent an immature or pre-processed form of mature TIGIT, and accordingly, included herein are mature or processed portions of the TIGIT polypeptide in SEQ ID NO: 1. In some embodiments, the TIGIT polypeptide is encoded by a polynucleotide comprising at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 10 or a fragment thereof.

In some embodiments, the expression of TIGIT is suppressed using a method comprising introducing into the NK cell a CRISPR/Cas endonuclease (Cas)9 system with a CRISPR/Cas guide RNA, wherein the guide RNA targets the TIGIT gene or a fragment thereof.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

“Guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. The gRNA described herein for targeting a TIGIT polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: SEQ ID NO: 11 or a fragment thereof. In some embodiments, the gRNA comprises a sequence set forth in SEQ ID NO: 11. In some examples, the TIGIT gRNA targets nucleotides 372-391 in the curated TIGIT RefSeq NM_173799 (i.e., nucleotide residues 372-391 of SEQ ID NO: 10), corresponding to amino acid residues 113-119 in the extracellular domain of the TIGIT protein UniProtKB/Swiss-Prot: Q495A1 (i.e., amino acid residues 113-119 of SEQ ID NO: 1).

Herein, “expression” means generation of mRNA by transcription from nucleic acids such as genes, polynucleotides, and oligonucleotides, or generation of a protein or a polypeptide by transcription from mRNA. “Suppression of expression” refers to a decrease of a transcription product or a translation product in a significant amount as compared with the case of no suppression.

The suppression of TIGIT expression herein shows, for example, a decrease of a transcription product or a translation product in an amount of about 10% or more, about 20% or more, about 30% or more, about 40% or more, about 50% or more, about 60% or more, about 70% or more, about 80% or more, about 90% or more, about 95% or more, or about 99% or more in comparison to the amount of transcription product or the translation product in an NK cell (e.g., a primary NK cell, a NK cell line, a non-expanded NK, or expanded NK) with no suppression of TIGIT.

In some embodiments, the engineered NK cell is suppressed in the expression of an inhibitory receptor. In some embodiments, the inhibitor receptor is selected from the group consisting of poliovirus receptor-related immunoglobulin domain-containing (PVRIG), CD96, lymphocyte activating 3 (LAG3), TIM-3, NKG2A, PD-1, and CTLA-4.

It should be understood that poliovirus receptor (PVR) (also termed CD155) is expressed on different types of tumor cells and is recognized by activating or inhibitory receptors on NK cells that exert opposite functions after interacting with this ligand. The activating receptor includes DNAM-1 (CD226) and inhibitory receptor receptors include TIGIT, CD96, and PVRIG. Accordingly, in some embodiments, the engineered NK cell described herein is further suppressed in the expression of an inhibitory receptor selected from the group consisting of CD96 and PVRIG.

“Poliovirus receptor-related immunoglobulin domain-containing (PVRIG)” refers herein to a polypeptide that, in humans, is encoded by the PVRIG gene. In some embodiments, the PVRIG polypeptide is that identified in one or more publicly available databases as follows: HGNC: 32190, NCBI Entrez Gene: 79037, Ensembl: ENSG00000213413, OMIM®: 617012, UniProtKB/Swiss-Prot: Q6DKI7. In some embodiments, the PVRIG polypeptide comprises the sequence of SEQ ID NO: 2, or a polypeptide sequence having at or greater than about 80%, about 85%, about 90%, about 95%, or about 98% homology with SEQ ID NO: 2, or a polypeptide comprising a portion of SEQ ID NO: 2. The PVRIG polypeptide of SEQ ID NO: 2 may represent an immature or pre-processed form of mature PVRIG, and accordingly, included herein are mature or processed portions of the PVRIG polypeptide in SEQ ID NO: 2.

“CD96” refers herein to a polypeptide that, in humans, is encoded by the CD96 gene. In some embodiments, the CD96 polypeptide is that identified in one or more publicly available databases as follows: HGNC: 16892, NCBI Entrez Gene: 10225, Ensembl: ENSG00000153283, OMIM®: 606037, UniProtKB/Swiss-Prot: P40200. In some embodiments, the CD96 polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. The CD96 polypeptide of SEQ ID NO: 3 may represent an immature or pre-processed form of mature CD96, and accordingly, included herein are mature or processed portions of the CD96 polypeptide in SEQ ID NO: 3.

“DNAX Accessory Molecule-1 (DNAM-1)” or “CD226” refers herein to a polypeptide that, in humans, is encoded by the CD226 gene. In some embodiments, the DNAM-1 polypeptide is that identified in one or more publicly available databases as follows: HGNC: 16961, NCBI Entrez Gene: 10666, Ensembl: ENSG00000150637, OMIM®: 605397, UniProtKB/Swiss-Prot: Q15762. In some embodiments, the DNAM-1 polypeptide comprises the sequence of SEQ ID NO: 4, or a polypeptide sequence having at or greater than about 80%, about 85%, about 90%, about 95%, or about 98% homology with SEQ ID NO: 4, or a polypeptide comprising a portion of SEQ ID NO: 4. The DNAM-1 polypeptide of SEQ ID NO: 4 may represent an immature or pre-processed form of mature DNAM-1, and accordingly, included herein are mature or processed portions of the DNAM-1 polypeptide in SEQ ID NO: 4.

“Lymphocyte activating 3 (LAG3)” refers herein to a polypeptide that, in humans, is encoded by the LAG3 gene. In some embodiments, the LAG3 polypeptide is that identified in one or more publicly available databases as follows: HGNC: 6476, NCBI Entrez Gene: 3902, Ensembl: ENSG00000089692, OMIM®: 153337, UniProtKB/Swiss-Prot: P18627. In some embodiments, the LAG3 polypeptide comprises the sequence of SEQ ID NO: 5, or a polypeptide sequence having at or greater than about 80%, about 85%, about 90%, about 95%, or about 98% homology with SEQ ID NO: 5, or a polypeptide comprising a portion of SEQ ID NO: 5. The LAG3 polypeptide of SEQ ID NO: 5 may represent an immature or pre-processed form of mature LAG3, and accordingly, included herein are mature or processed portions of the LAG3 polypeptide in SEQ ID NO: 5.

“PD-1” refers herein to a polypeptide that, in humans, is encoded by the PDCD1 gene. In some embodiments, the PD-1 polypeptide is that identified in one or more publicly available databases as follows: HGNC: 8760 NCBI Entrez Gene: 5133 Ensembl: ENSG00000188389 OMIM®: 600244 UniProtKB/Swiss-Prot: Q15116. In some embodiments, the PD-1 polypeptide comprises the sequence of SEQ ID NO: 6, or a polypeptide sequence having at or greater than about 80%, about 85%, about 90%, about 95%, or about 98% homology with SEQ ID NO: 6, or a polypeptide comprising a portion of SEQ ID NO: 6. The PD-1 polypeptide of SEQ ID NO: 6 may represent an immature or pre-processed form of mature PD-1, and accordingly, included herein are mature or processed portions of the PD-1 polypeptide in SEQ ID NO: 6.

“CTLA-4” refers herein to a polypeptide that, in humans, is encoded by the CTLA4 gene. In some embodiments, the CTLA-4 polypeptide is that identified in one or more publicly available databases as follows: HGNC: 2505, NCBI Entrez Gene: 1493, Ensembl: ENSG00000163599, OMIM®: 123890, UniProtKB/Swiss-Prot: P16410. In some embodiments, the CTLA-4 polypeptide comprises the sequence of SEQ ID NO: 7, or a polypeptide sequence having at or greater than about 80%, about 85%, about 90%, about 95%, or about 98% homology with SEQ ID NO: 7, or a polypeptide comprising a portion of SEQ ID NO: 7. The CTLA-4 polypeptide of SEQ ID NO: 7 may represent an immature or pre-processed form of mature CTLA-4, and accordingly, included herein are mature or processed portions of the CTLA-4 polypeptide in SEQ ID NO: 7.

“TIM-3” refers herein to a polypeptide that, in humans, is encoded by the HAVCR2 gene. In some embodiments, the TIM-3 polypeptide is that identified in one or more publicly available databases as follows: HGNC: 18437, NCBI Entrez Gene: 84868, Ensembl: ENSG00000135077, OMIM®: 606652, UniProtKB/Swiss-Prot: Q8TDQ0. In some embodiments, the TIM-3 polypeptide comprises the sequence of SEQ ID NO: 8, or a polypeptide sequence having at or greater than about 80%, about 85%, about 90%, about 95%, or about 98% homology with SEQ ID NO: 8, or a polypeptide comprising a portion of SEQ ID NO: 8. The TIM-3 polypeptide of SEQ ID NO: 8 may represent an immature or pre-processed form of mature TIM-3, and accordingly, included herein are mature or processed portions of the TIM-3 polypeptide in SEQ ID NO: 8.

“NKG2A” refers herein to a polypeptide that, in humans, is encoded by the KLRC1 gene. In some embodiments, the NKG2A polypeptide is that identified in one or more publicly available databases as follows: HGNC: 6374 NCBI Entrez Gene: 3821 Ensembl: ENSG00000134545 OMIM®: 161555 UniProtKB/Swiss-Prot: P26715. In some embodiments, the NKG2A polypeptide comprises the sequence of SEQ ID NO: 9, or a polypeptide sequence having at or greater than about 80%, about 85%, about 90%, about 95%, or about 98% homology with SEQ ID NO: 9, or a polypeptide comprising a portion of SEQ ID NO: 9. The NKG2A polypeptide of SEQ ID NO: 9 may represent an immature or pre-processed form of mature NKG2A, and accordingly, included herein are mature or processed portions of the NKG2A polypeptide in SEQ ID NO: 9.

Methods of increasing expression levels of a polypeptide are known in the art, comprising, for example, introducing and expressing genes into the cell. In the context of an expression vector, the vector can be readily introduced into a host cell, e.g., mammalian, bacterial, yeast, or insect cell by any method in the art. For example, the expression vector can be transferred into a host cell by physical, chemical, or biological means. See, e.g., WO2012079000A1, incorporated by reference herein in its entirety.

In some embodiments, the NK cell described herein is a primary NK cell or a NK cell line. In some embodiments, the NK cell described herein is an expanded NK cell or a non-expanded NK cell. In some embodiments, the NK cell expanding composition comprises a feeder cell, an engineered PM particle, or an exosome. In some embodiments, the feeder cell or engineered particle comprises an Fc domain bound to an external surface thereof. In some embodiments, the NK cell expanding composition further comprises an NK cell effector agent.

### (I) Engineered Feeder Cells, Engineered Plasma Membrane Particles and Engineered Exosomes Comprising Membrane Bound Fc

Compositions according to the disclosure include compositions comprising Fc-bound feeder cells (FCs), compositions comprising Fc-bound engineered plasma membrane (PM) particles, and compositions comprising Fc-bound engineered exosomes. Fc-bound engineered PM particles include PM nanoparticles derived from Fc-bound feeder cells. Fc bound engineered exosomes include exosomes or other extracellular vesicles derived from Fc-bound feeder cells, as also described in further detail below. Alternatively, exosomes may be derived from other sources such as platelets and megakaryocytes.

As used herein, the term “Fc-bound” shall be understood as referring to the coupling of an Fc domain in an inverted orientation (i.e., the amino terminal end facing intracellularly) to the external surface of a feeder cell or engineered particle via a transmembrane peptide. This can be achieved using the Fc fusion peptides disclosed herein. Thus, one aspect of the present disclosure provides a feeder cell composition comprising at least one Fc-bound feeder cell, i.e., a feeder cell comprising an Fc domain bound to an external surface of the feeder cell, as described in further detail below. For example, a feeder cell can be genetically modified to express an Fc domain bound to an external surface of the feeder cell, i.e., to express an Fc fusion peptide as described further below. Another aspect of the disclosure provides an NK cell expanding composition free of feeder cells, comprising at least one Fc-bound engineered particle, i.e., an engineered particle comprising an Fc domain bound in inverted orientation to an external surface of the feeder cell. In some aspect, the feeder cells can be engineered to express a ligand that can be tagged with a humanized antibody.

In a feeder cell composition, the at least one Fc-bound feeder cell optionally comprises at least one cell NK cell effector agent. In one example, an Fc-bound feeder cell comprises one cell NK cell effector which is IL-15 or IL-21. Fc-bound feeder cells can comprise at least two or more different NK cell effector agents.

In an NK cell expanding composition free of feeder cells, Fc-bound engineered PM particles optionally comprise at least one cell NK cell effector agent. In one example, an Fc-bound engineered particle comprises one cell NK cell effector which is IL-15 or IL-21. Fc-bound engineered PM particles can comprise at least two or more different NK cell effector agents.

In either a feeder cell composition, or a composition free of feeder cells, in which at least two NK cell effector agents are present, the second NK cell effector agent can for example be 41BBL. In either a feeder cell composition, or an NK cell expanding composition free of feeder cells, in which the feeder cells or engineered PM particles comprise one or more NK cell effector agents, NK cell effector agents can be selected from 41BBL, IL-15, IL-2, IL-12, IL-18, IL-21, MICA, UBLP, 2B4, LFA-1, a Notch ligand, ligands for NKp46, or BCM1/SLAMF2, TLR ligands, and NKG2D ligands, or a cytokine. In an exemplary such composition, at least one additional NK cell effector agent is IL-15 or IL-21. In some embodiments, the NK cell effector agents can be selected from IL-12, IL-15, and IL-18.

### (a) Fc-Bound Feeder Cells

The present disclosure provides feeder cells comprising an Fc fusion peptide as detailed above. NK cell feeder cells for use in the methods disclosed herein, and for use in making the PM particles and exosomes disclosed herein, can be either irradiated autologous or allogeneic peripheral blood mononuclear cells (PBMCs) or nonirradiated autologous or allogeneic PBMCs, RPMI8866, HFWT, 721.221 or K562 cells as well as EBV-LCLs, other non-HLA or low-HLA expressing cell lines or patient derived primary tumors which can be used as a tumor vaccine. Fc-bound feeder cells can be prepared by transfecting or transducing feeder cells with any Fc fusion peptide as described herein, using standard transduction or transfection techniques well known in the art. For example, cDNA vectors for Fc fusion peptides disclosed herein can be ligated into an expression plasmid, which allows expression in bacterial (E. coli), insect, or mammalian cells. The cDNA vector can be FLAG- or HIS-tagged. Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, NY, 3rd edition, 2001). Alternatively, molecules can be introduced into a cell by microinjection. For example, molecules can be injected into the cytoplasm or nuclei of the cells of interest. The amount of each molecule introduced into the cell can vary, but those skilled in the art are familiar with means for determining the appropriate amount.

It will be understood that various molecules can be introduced into a cell simultaneously or sequentially. For example, an Fc fusion peptide and one or more membrane bound NK cell effector agents can be introduced to a feeder cell at the same time. Alternatively, one can be introduced first and then the other molecule(s) can later be introduced into the cell. For example, feeder cells once having been transfected or transduced with an Fc fusion peptide can be further transfected with membrane bound NK cell effector agents such as IL-15 and/or IL-21 and/or 41BBL and/or infected as an EBV-LCL and/or other NK cell effector agent(s). Alternatively, feeder cells can be simultaneously transfected or transduced with an Fc fusion peptide and membrane bound NK cell effector agents such as IL-15 and/or IL-21 and/or 41BBL and/or EBV-LCL and/or other NK cell effector agent(s). Alternatively, feeder cells previously transfected or transduced and expressing membrane bound NK cell effector agents such as IL-15 and/or IL-21 and/or 41BBL and/or infected as an EBV-LCL and/or other NK cell effector agent(s), can be transfected or transduced with an Fc fusion peptide. It will be also appreciated that other means such as chemical conjugation methods known in the art can be used to achieve a membrane bound Fc.

In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

Fc-bound feeder cells can be used in cell culture to stimulate NK cells directly, or can be used to prepare PM particles or exosomes derived from the feeder cells.

### (b) Fc-Bound PM Particles

Fc-bound engineered PM particles include Fc-bound PM particles, which can be prepared from Fc-bound NK cell feeder cells using well known methods. PM particles are vesicles made from the plasma membrane of a cell or artificially made (e.g., liposomes). A PM particle can contain a lipid bilayer or simply a single layer of lipids. A PM particle can be prepared in single lamellar, multi-lamellar, or inverted form. PM particles can be prepared from Fc-bound feeder cells as described herein, using known plasma membrane preparation protocols or protocols for preparing liposomes such as those described in U.S. Pat. No. 9,623,082, the entire disclosure of which is herein incorporated by reference. In certain aspects, PM particles as disclosed herein range in average diameter from about 170 to about 300 nm.

### (c) Fc-Bound Exosomes

Fc-bound exosomes as disclosed herein can be prepared from exosome-secreting cells, which can be prepared from Fc-bound NK cell feeder cells using well known methods, wherein the exosome is an extracellular product of exosome-secreting cells, as described in United States Pat. App. Pub. No. 20170333479, the entire disclosure of which is herein incorporated by reference. Exosomes comprise lipids and proteins and the identity of the proteins found in a particular exosome is dependent on the cell(s) that produced them. Exosomes disclosed herein comprise an Fc fusion peptide as disclosed herein (i.e., are Fc-bound), and optionally one or more stimulatory peptides (NK cell effector agents) present in the exosome membrane. Exosomes can be produced for example from cell lines engineered for improved formation or release of exosomes. Such cell lines include, but are not limited to, Fc-bound cell lines as described above in Section I(a). Non-limiting cell lines are Fc-bound K562-mb15-41BBL and Fc-bound K562. In certain aspects, exosomes as disclosed herein range in average diameter from about 30 to about 100 nm, or to about 160 nm. In one aspect, exosomes average about 60-80 nm in diameter. The ability with exosomes to achieve particle sizes smaller than readily achieved with PM particles means that exosomes can be more readily adapted to uses where a smaller size is preferable. For example, exosomes may be preferred in applications requiring diffusion through physiological barriers, enhanced biodistribution through tissue compartments, or intravenous injections.

In some embodiments, the NK cell expanding composition disclosed herein is combined with a cell medium solution comprising at least one soluble media component such as a cytokine, IL-2, IL-12, IL-15, IL-18, IL-21, NAM, ascorbate or any combination thereof.

In some examples, The NK cell expanding compositions used herein and the methods for NK cell expansion described herein are those described in U.S. Pat. Pub. No. 20200237822, the entire disclosure of which is herein incorporated by reference.

### Compositions and Methods

Also disclosed herein is a method of treating, decreasing, inhibiting, reducing, ameliorating, and/or preventing a cancer, metastasis, or an infectious disease in a subject comprising administering to the subject a therapeutically effective amount of the engineered NK cell described herein, wherein the engineered NK cell is suppressed in the expression of T Cell Immunoreceptor with Ig and ITIM Domains (TIGIT). Similarly, the present disclosure provides a therapeutically effective amount of the engineered NK cells disclosed herein for use in treating, decreasing, inhibiting, reducing, ameliorating, and/or preventing a cancer, metastasis, or an infectious disease in a subject, wherein the engineered NK cell is suppressed in the expression of T Cell Immunoreceptor with Ig and ITIM Domains (TIGIT).

Also disclosed herein is a method of treating, decreasing, inhibiting, reducing, ameliorating, and/or preventing a cancer, metastasis, or an infectious disease in a subject comprising administering to the subject a therapeutically effective amount of an NK cell and a therapeutically effective amount of a T Cell Immunoreceptor with Ig and ITIM Domains (TIGIT) inhibitor. Similarly, the present disclosure provides a therapeutically effective amount of an NK cell and a therapeutically effective amount of a TIGIT inhibitor for use in treating, decreasing, inhibiting, reducing, ameliorating, and/or preventing a cancer, metastasis, or an infectious disease in a subject. In some embodiments, the NK cell is a primary NK cell or a NK cell line. In some embodiments, the NK cell is an expanded NK cell. In some embodiments, the expanded NK cell is exposed in vitro/in vivo to the NK cell expanding composition disclosed herein (for example, a feeder cell, an engineered PM particle, or an exosome). In some embodiments, the feeder cell or engineered particle comprises an Fc domain bound to an external surface thereof. In some embodiments, the NK cell expanding composition further comprises an NK cell effector agent. NK cell effector agents can be selected from 41BBL, IL-15, IL-2, IL-12, IL-18, IL-21, MICA, UBLP, 2B4, LFA-1, a Notch ligand, ligands for NKp46, or BCM1/SLAMF2, TLR ligands, NKG2D ligands, and a cytokine. In an exemplary such composition, at least one NK cell effector agent is IL-15 or IL-21. In some embodiments, the NK cell effector agents can be selected from IL-12, IL-15, and IL-18. In some embodiments, the NK cell effector agent comprises IL-21 and/or 41BBL.

In some embodiments, the method described herein further comprises administering to the subject a therapeutically effective amount of a TIGIT inhibitor. In some embodiments, the TIGIT inhibitor is an anti-TIGIT antibody (e.g., tiragolumab, vibostolimab, domvanalimab, BMS-986207, etigilimab, EOS-448, COM902, ASP8374, SEA-TGT, BGB-A1217, IBI-939, or M6223).

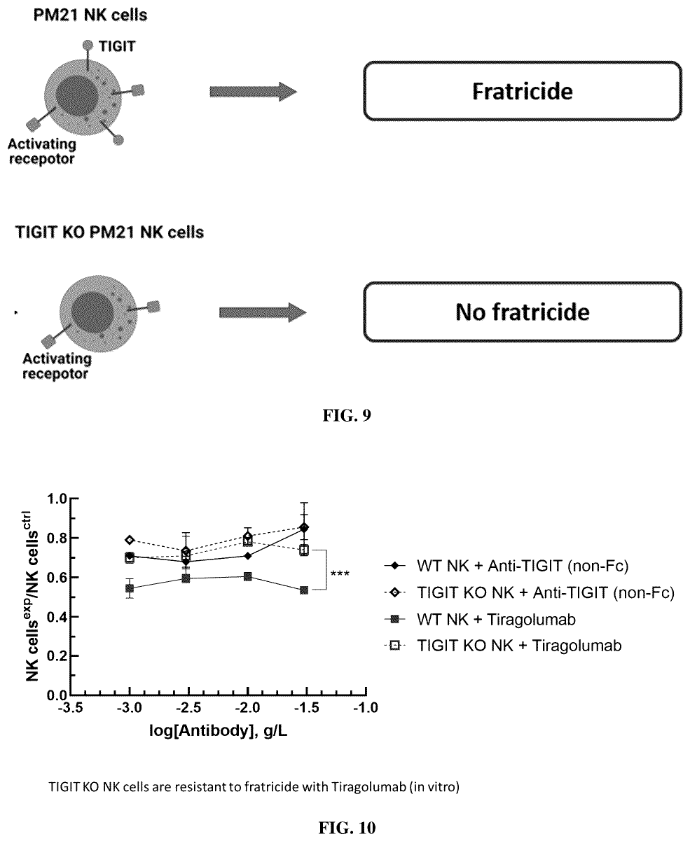

It should be understood that many anti-TIGIT therapies (e.g., anti-TIGIT antibodies) are in development. Fc-competent therapeutic antibodies targeting TIGIT can induce NK cell fratricide in NK cells expressing TIGIT. TIGIT knockout NK cells can prevent fratricide and restore NK cell numbers. This is illustrated by the schematic shown, for example, in FIG. 9. Accordingly, in some embodiments, the method disclosed herein further comprises administering to the subject a therapeutically effective amount of a TIGIT inhibitor. In some embodiments, the TIGIT inhibitor is an anti-TIGIT antibody. In some embodiments, the anti-TIGIT antibody comprises a fragment crystallizable region (Fc region) that binds to an Fc receptor (e.g., CD16). Such bindings of the Fc region to the Fc receptor may trigger effector functions of the immune system (e.g., ADCC). In some embodiments, the anti-TIGIT antibody lacks a Fc region or comprises a Fc region having a reduced affinity to an Fc receptor (e.g., CD16) relative to a reference control. In some examples, the anti-TIGIT antibody comprises one or more mutations on the Fc region that reduce the binding affinity of the Fc region to the Fc receptor.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with TIGIT such that TIGIT is inhibited from interacting with its receptor. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, nanobodies, and the like, including hybrid fragments. Thus, fragments of the antibodies that retain the ability to bind their specific antigens are provided. For example, fragments of antibodies which maintain TIGIT binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response. As will be understood by those in the art, the immunological binding reagents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof. The term “antibody” is thus used to refer to any antibody-like molecule that has an antigen binding region, and this term includes antibody fragments that comprise an antigen binding domain such as Fab′, Fab, F(ab′)₂, single domain antibodies (DABs), T and Abs dimer, Fv, scFv (single chain Fv), dsFv, ds-scFv, Fd, linear antibodies, nanobodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics.

In some embodiments, the method further comprises administering to subject a therapeutically effective amount of a checkpoint blockade. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). In some embodiments, the checkpoint blockade comprises a PD-1 inhibitor, a PD-L1 inhibitor, a PD-L2 inhibitor, or a CTLA-4 inhibitor.

A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, and eye

It is intended herein that the disclosed methods of inhibiting, reducing, and/or preventing cancer metastasis and/or recurrence can comprise the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride , Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, , Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab). It is also intended herein that the disclosed uses of the disclosed compositions and/or an engineered NK cell population for inhibiting, reducing, and/or preventing cancer metastasis and/or recurrence can comprise use in combination the use of any anti-cancer agent known in the art including, but not limited to those agents listed above.

In some aspects, the engineered NK cells and uses of the cells all as disclosed herein are for treating an infectious disease caused by a viral infection, wherein the viral infection comprises an infection of Herpes Simplex virus- 1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

Alternatively, in any of the therapeutic methods or uses for treatment, the additional therapeutic agent can be an antiviral agent selected from but not limited to a 5-substituted 2-deoxyuridine analog, a nucleoside analogs, a (nonnucleoside) pyrophosphate analog, a nucleoside reverse transcriptase (RT) inhibitors (NRTI), a nonnucleoside reverse transcriptase inhibitor (NNRTI), a protease inhibitor (PI), and integrase inhibitor, an entry inhibitor, and acyclic guanosine analog, an acyclic nucleoside phosphonate (ANP) analog, a hepatitis C virus (HCV) NS5A and NS5B inhibitor, and influenza virus inhibitor, an immunostimulator, an interferon, an oligonucleotide, and an antimitotic inhibitor. Non-limiting examples of antiviral agents are acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; leveteracetam; tramadol; or any of their isomers tautomers, analogs, polymorphs, solvates, derivatives, or pharmaceutically acceptable salts.

In some aspects, the engineered NK cells and uses of the cells all as disclosed herein are for treating infectious disease caused by a bacterial infection, wherein the bacterial infection comprises an infection of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species.

In some aspects, the engineered NK cells and uses of the cells all as disclosed herein are for treating infectious disease caused by a fungal infection, wherein the fungal infection comprises an infection of Candida albicans, Cryptococcus neoformans, Histoplama capsulatum, Aspergillus fumigatus, Coccidiodes immitis, Paracoccidioides brasiliensis, Blastomyces dermitidis, Pneumocystis carinii, Penicillium marneffi, or Alternaria alternate.

In some aspects, the engineered NK cells and uses of the cells all as disclosed herein are for treating infectious disease caused by a parasitic infection, wherein the parasitic infection comprises an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

Alternatively, in any of the methods or uses the additional therapeutic agent can be an antibiotic agent selected from but not limited to penicillin, tetracycline, cephalosporin, lincomycin, a macrolide, a sulfonamide, a glycopeptide, an aminoglycosides, and a carbapenem. Non-limiting examples of antibacterial agents are amoxicillin, doxycycline, cephalexin, ciprofloxacin, clindamycin, metronidazole, azithromycin, sulfamethoxazole and trimethoprim, clavulanate, and levofloxacin.

In some embodiments, the engineered NK cells administered or used in any of the methods for reactivating an NK cell, reversing NK cell exhaustion, and/or enhancing NK cell function disclosed herein and/or methods of treating, decreasing, inhibiting, reducing, ameliorating, and/or preventing a cancer, metastasis, and/or infectious disease disclosed herein are formulated in a pharmaceutically acceptable carrier and a pharmaceutically acceptable excipient.

As the timing of a cancer, metastatic condition, or infection can often not be predicted, it should be understood the disclosed methods of treating, preventing, reducing, and/or inhibiting a cancer, metastatic condition, or infection, or the use of any of the disclosed compositions or combinations for such treating, preventing, reducing, and/or inhibiting of a cancer, metastatic condition, or infection, can be practiced prior to or following the onset of the cancer, metastatic condition, or infection, to treat, prevent, inhibit, and/or reduce the muscular disease.

NK cell exhaustion is observed in subjects having cancers or certain infectious diseases. The engineered NK cells disclosed herein shows enhanced function (including, for example, enhanced cytotoxicity function and/or increased expression of IFNγ, TNFα, and/or CD107a). Accordingly, disclosed herein is a method for reactivating an NK cell, reversing NK cell exhaustion, and/or enhancing NK cell function, said method comprising suppressing the expression of TIGIT of the NK cell or incubating the NK cell with a TIGIT inhibitor. The disclosed methods have the added benefit of providing cells with higher cytotoxicity and/or ADCC functionality. In some examples, the engineered NK cell exhibits about 2× the cytotoxicity or expression of the cytokines, at least about 5× the cytotoxicity or expression of the cytokines, or at least about 10× the cytotoxicity or expression of the cytokines of NK cells that are not manipulated to suppress the expression of TIGIT. In some embodiments, the unmanipulated NK cells are exhausted NK cells. In some embodiments, the exhausted NK cells have increased levels of one or more of PD-1, TIGIT, LAG3, and TIM3. In some embodiments, the exhausted NK cells have decreased levels of Ki67, IFNγ, TNFα, and/or CD107a. In some embodiments, the unmanipulated NK cells are primary NK cells derived from a subject (e.g., a healthy person or a cancer patient). In some embodiments, the NK cell is an expanded NK cell or a non-expanded NK cell. In some embodiments, the expanded NK cell is exposed in vitro or ex vivo to an NK cell expanding composition (e.g., a feeder cell, engineered PM particle, or exosome disclosed herein). The manipulated NK cells can be administered to a subject having a cancer or a certain infectious disease.

In some aspects, disclosed herein is a method of treating, decreasing, inhibiting, reducing, ameliorating, and/or preventing a cancer, metastasis, or an infectious disease in a subject comprising 1) obtaining an NK cell; 2) reactivating the NK cell, reversing exhaustion of the NK cell, and/or enhancing function of the NK cell in vitro or ex vivo by suppressing the expression of TIGIT of the NK cell or contacting the NK cell with a TIGIT inhibitor; and 3) administering a therapeutically effective amount of the NK cells to the subject. In some embodiments, the NK cell of step a) is an exhausted NK cell. In some embodiments, the exhausted NK cell has increased levels of one or more of PD-1, TIGIT, LAG3, and TIM3. In some embodiments, the exhausted NK cell has decreased levels of Ki67, IFNγ, TNFα, and/or CD107a. In some embodiments, the NK cell of step a) is a primary NK cell derived from a subject (e.g., a healthy person or a cancer patient). In some embodiments, the methods for reactivating an NK cell, reversing NK cell exhaustion, and/or enhancing NK cell function disclosed herein and/or methods of treating, decreasing, inhibiting, reducing, ameliorating, and/or preventing a cancer, metastasis and/or infectious disease disclosed herein further comprises administering to the subject a TIGIT inhibitor. In some embodiments, said methods can further comprises administering to subject a therapeutically effective amount of a checkpoint blockade. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). In some embodiments, the checkpoint blockade comprises a PD-1 inhibitor, a PD-L1 inhibitor, a PD-L2 inhibitor, or a CTLA-4 inhibitor.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1. TIGIT Blockade Improves Anti-Tumor Activity of Ex Vivo Expanded NK Cells

In this study, TIGIT signaling was examined in the context of ex vivo expanded NK cells and the effect of TIGIT blockade on the anti-tumor activities of NK cells was evaluated.

Methods. NK cells were activated overnight with cytokines or ex vivo expanded with PM21-particles. TIGIT expression was determined on NK cells with qRT-PCR and flow cytometry. Cytotoxicity was assessed by kinetic, imaging-based assay (Incucyte S3) with A549 and NCI-H1299 cells cultured in 3D. Cytotoxicity was calculated based on untreated controls at different time-points. Results from two multiple donors were normalized to cytotoxicity of NK cells with isotype for individual donors and was compared to the cytotoxicity of NK cells with anti-TIGIT. Unpaired t test was used to determine statistical significance. PVR+K562 cells, stably expressing PVR, were used to restimulate A549 spheroid-exposed NK cells to measure IFNγ, TNFα and surface CD107a. Furthermore, phenotypic changes of NK cells upon TIGIT blockade were examined by analyzing a set of activating and inhibitory receptors by flow cytometry.

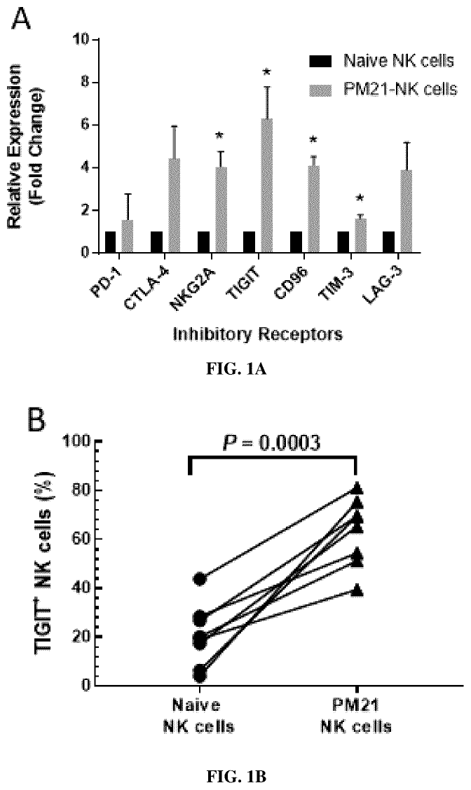

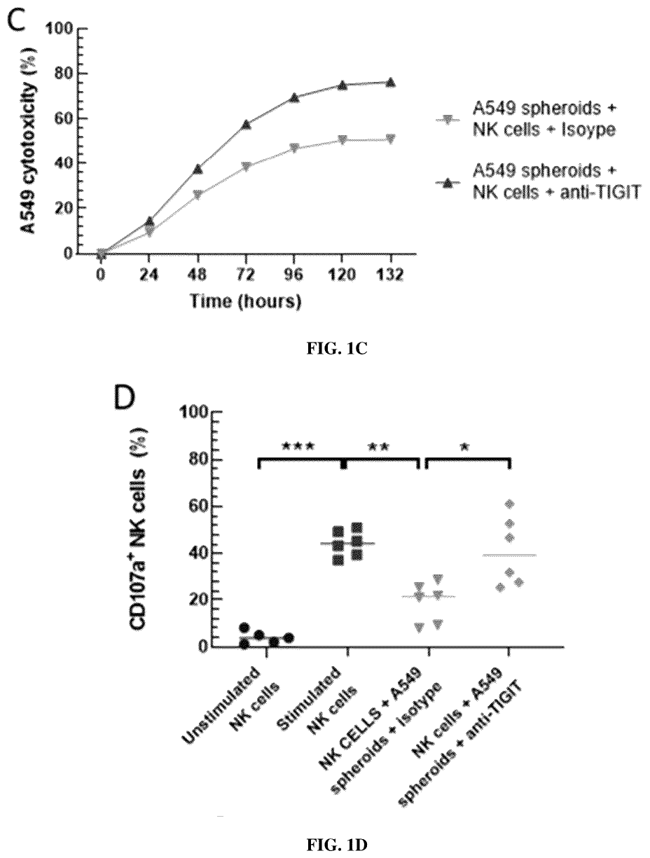

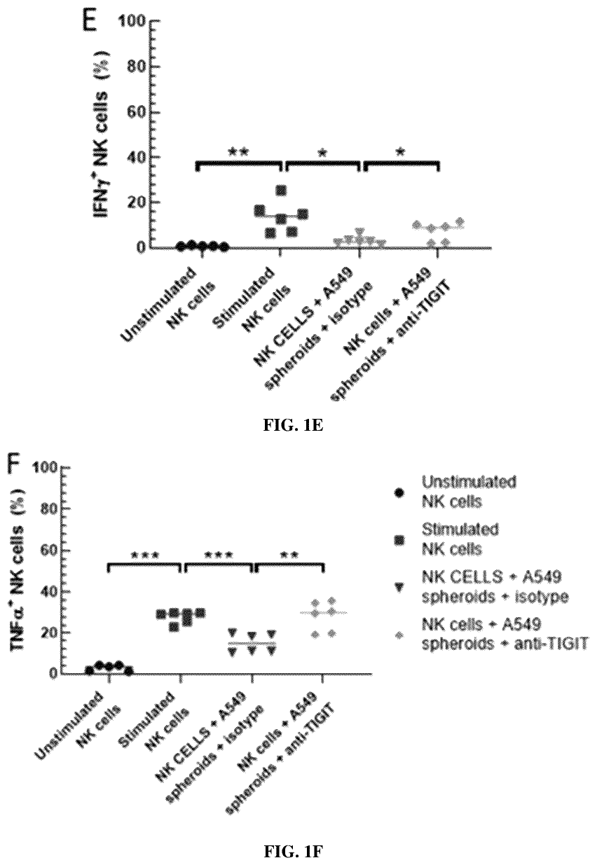

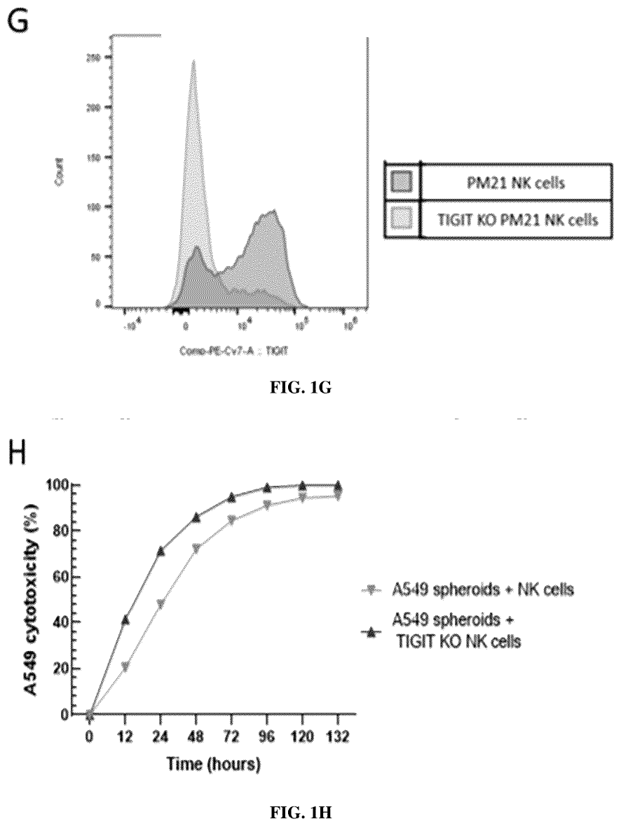

Results: The effect of NK cell expansion/activation on TIGIT expression was assessed. Transcriptomic analysis showed that TIGIT is upregulated on either expanded NK cells or cytokine-activated NK cells both at the mRNA and protein level. The effect of TIGIT blockade on NK cell cytotoxicity was examined by co-culturing ex vivo expanded NK cells with cancer cells in the presence of anti-TIGIT antibodies or respective isotypes. TIGIT blockade significantly increased cytotoxicity of ex vivo expanded NK cells against A549 (1.3 fold, P<0.0001) and NCI-H1299 (1.29 fold, P=0.0003) spheroids after 48 h. To access exhaustion, NK cells exposed to A549 spheroids for 7 days were restimulated with PVR+K562 cells. TIGIT blockade prevented NK cell exhaustion resulting in increased expression of IFNγ, TNFα and surface CD107a on restimulated NK cells (FIGS. 1D-1F). TIGIT blockade did not result in any significant change of the expression of inhibitory and activating receptors on ex vivo expanded NK cells.

Conclusion: TIGIT is highly expressed on ex vivo expanded and cytokine-activated NK cells. TIGIT blockade with anti-TIGIT antibodies significantly improves anti-tumor activities of ex vivo expanded NK cells. Thus, ex vivo expanded NK cells and anti-TIGIT antibodies have translational potential as a promising combination therapy to improve overall anti-tumor activity.

### Example 2. Investigation of TIGIT Signaling in PM21 Expanded NK Cells to Improve their Anti-Tumor Response

TIGIT signaling in Treg cells increases inhibitory IL-10 and Fg12 expression and enhances their immunosuppressive properties. Previous studies also observed that TIGIT is upregulated on NK cells in cancers and often correlates with their exhaustion. In preclinical mouse models, blockade of TIGIT restored NK cell cytotoxicity and increased IFNγ and TNFα expression, and promoted tumor-specific T cell immunity. Anti-TIGIT in combination with anti-PD-L1 further enhanced anti-tumor activity and this effect depended on the presence of NK cells. Anti-TIGIT antibodies are now showing results in both preclinical models and human trials. In preclinical mouse models, anti-TIGIT antibodies alone or in combination with checkpoint inhibitors such as anti-PD-1/anti-PD-L1/anti-TIM-3 prevent tumor growth and increase their survival. A Phase II clinical trial (NCT03563716) by Genentech examined that anti-TIGIT in combination with anti-PD-L1 shows a higher overall response rate (55.2%) compared to anti-TIGIT alone (17.2%) in PD-L1 expressing non-small cell lung cancer (NSCLC) patients. Therapeutic efficacy of anti-TIGIT antibodies depends on multiple mechanisms including the restoration of T cell effector function and the ADCC dependent depletion of Treg cells. Although there are multiple preclinical and clinical studies combining anti-TIGIT with other checkpoint inhibitors, the efficacy of adoptive NK cells and anti-TIGIT as combination therapy is yet to be examined.

NK cells are a component of the innate immune system that comprise only 5-10% of total peripheral blood lymphocytes and genetically modifying NK cells remains a challenge, which makes it difficult to collect enough NK cells as a therapy. Several methods are currently being used to expand NK cells including cytokine and feeder cell-based expansion methods. However, these methods have some disadvantages. For example, cytokine-based methods cannot expand NK cells significantly. Furthermore, feeder cell expanded NK cells need to be purified after expansion as the presence of any residual feeder cells may create a potential risk of further malignancies in cancer patients. A particle (PM21) based NK cell expansion method was developed that significantly expands clinical-grade cytotoxic NK cells without the requirement of further purification. These NK cells exhibit robust antitumor activity both in vitro and in vivo. Previous studies demonstrated that PM21-NK cells induced PD-L1 expression on cancer cells both in vitro and in vivo. PD-L1 blockade enhanced anti-tumor activity and persistence of PM21-NK cells in vivo and extended the survival of tumor-bearing mice, although PM-21 NK cells are mostly PD-1 negative. These findings indicate that PD-L1 blockade can, directly and indirectly, enhance the anti-tumor response of PM21-NK cells.

This example 1) investigates the role of TIGIT signaling in PM21 NK cells and evaluates the potential of anti-TIGIT and PM21 NK cells as a combination therapy; and 2) shows that modifying TIGIT signaling in PM21 NK cells enhances their anti-tumor activity. The data herein show that TIGIT is highly expressed on NK cells and the blockade of TIGIT improved cytotoxicity against TIGIT ligand+ cancer spheroids and increased expression of IFNγ, TNFα, and surface CD107a. The data herein also show that TIGIT deletion in PM21 NK cells increased cytotoxicity against TIGIT ligand+ cancer spheroids. These data indicate that blockade of TIGIT signaling enhances anti-tumor activity of PM21 NK cells by preventing their exhaustion. This example shows how TIGIT signaling regulates PM21 NK cell properties and supports the use of engineered PM21 NK cells with suppressed TIGIT expression and other combination therapies to improve anti-tumor activity.

The analysis herein identified that PM21 NK cells express inhibitory receptors including TIGIT, CD96, NKG2A, TIM-3, and LAG-3, with TIGIT being the most upregulated receptor among them. The data provided herein show that the blockade or TIGIT deletion increased the cytotoxicity of PM21 NK cells and enhanced the expression of IFNγ, TNFα, and surface CD107a.

This example analyzed the role of TIGIT signaling in PM21 NK cells, leading to the development of strategies to improve the effector function of PM21 NK cells. TIGIT signaling in NK cells was blocked to analyze its effect on the phenotypic, metabolic, and functional properties of these NK cells. Transcriptome analysis was conducted to understand how TIGIT signaling regulates human NK cells. This example not only determined the functional consequence of TIGIT signaling in NK cells, but also helped to develop modified NK cells and combination immunotherapy approaches with better therapeutic efficacy against cancer cells.

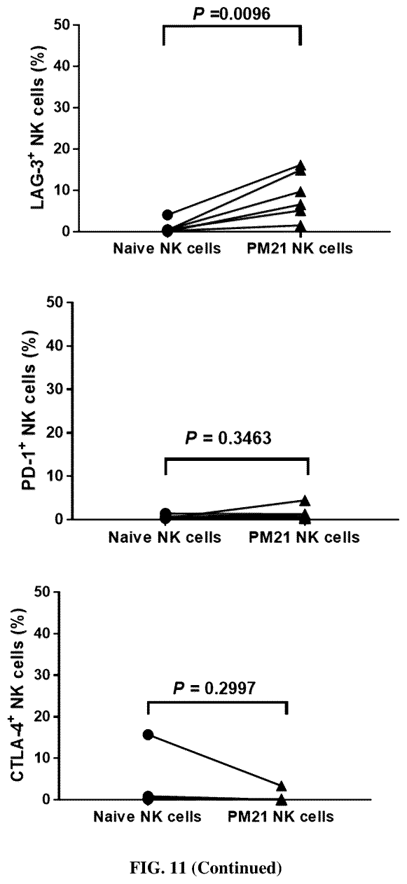

To understand the expression of inhibitory receptors on PM21 NK cells, a set of inhibitory receptors including PD-1, CTLA-4, TIGIT, NKG2A, TIM-3, CD96, and LAG-3 were analyzed by measuring RNA expression with qRT-PCR and protein expression with flow cytometry. In these studies, significant upregulation of multiple inhibitory receptors including TIGIT, NKG2A, TIM-3, CD96, and LAG-3 was observed at both the RNA (FIG. 1A; FIG. 11) and protein level. Among them, TIGIT was the most upregulated in NK cells (FIG. 1B). However, no significant protein expression of PD-1 or CTLA-4 was observed (FIG. 11). Based on this expression analysis, TIGIT, which is a major inhibitory receptor of NK cells, were selected for further studies. Previous studies showed that blockade of TIGIT signaling restores NK cell anti-tumor activity in mouse models. To understand the effect of TIGIT blockade on cytotoxicity and functional properties of PM21 NK cells, PM21 NK cells were co-cultured with TIGIT ligand+A549 3D lung cancer spheroids for 7 days and then were analyzed with a live imaging system. In addition to a cytotoxicity assay, the functional phenotypes of PM21 NK cells were further analyzed. These experiments showed that the blockade of TIGIT on PM21 NK cells with anti-TIGIT antibodies significantly enhanced the cytotoxicity of PM21 NK cells against A549 3D lung cancer cell spheroids (FIG. 1C). The blockade of TIGIT signaling also enhanced the expression of IFNγ, TNFα, and surface CD107a against A549 cell spheroids (FIGS. 1D, 1E, and 1F).

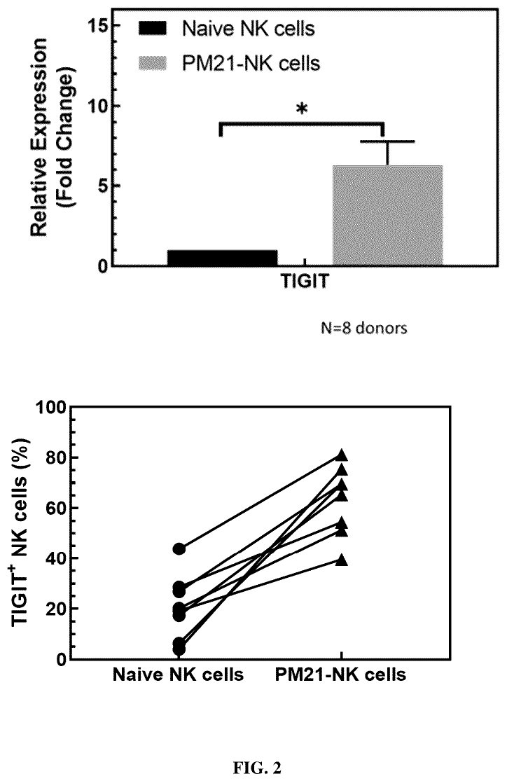

1) Investigate the role of TIGIT signaling in PM21 NK cells and evaluate anti-TIGIT and PM21 NK cells as a combination therapy. It was observed that PM21 NK cells significantly upregulate TIGIT compared to naïve or unexpanded NK cells (FIG. 1B and FIG. 2). Here, it is shown that inhibitory TIGIT signaling promotes the exhaustion of PM21 NK cells, and the blockade of this receptor can restore the anti-tumor properties of PM21 NK cells against TIGIT ligand+ cancer cells (FIG. 1). For this study, TIGIT signaling on PM21 NK cells was blocked to determine TIGIT signaling regulated phenotypic, functional, and metabolic properties involved in NK cell exhaustion. Furthermore, transcriptome analysis was performed to identify signaling pathways regulated by TIGIT signaling. Finally, the combination of anti-TIGIT and PM21 NK cells was studied to determine it the combination can improve anti-tumor activity.

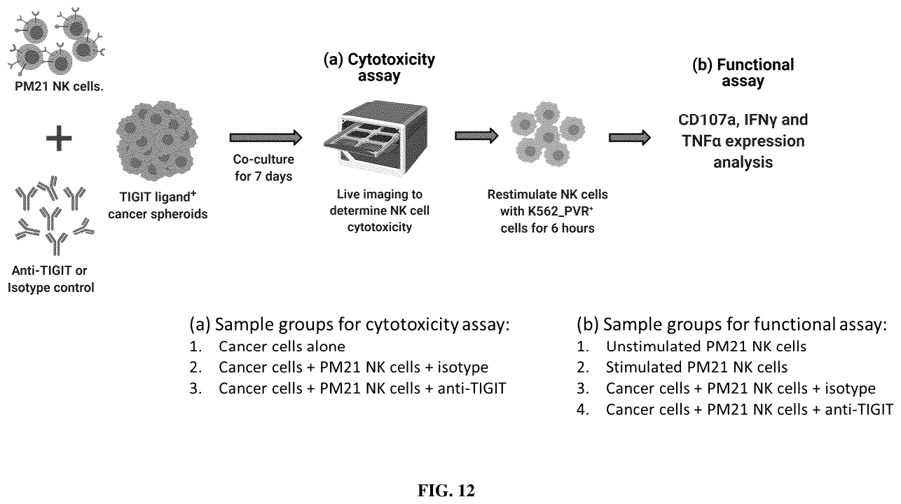

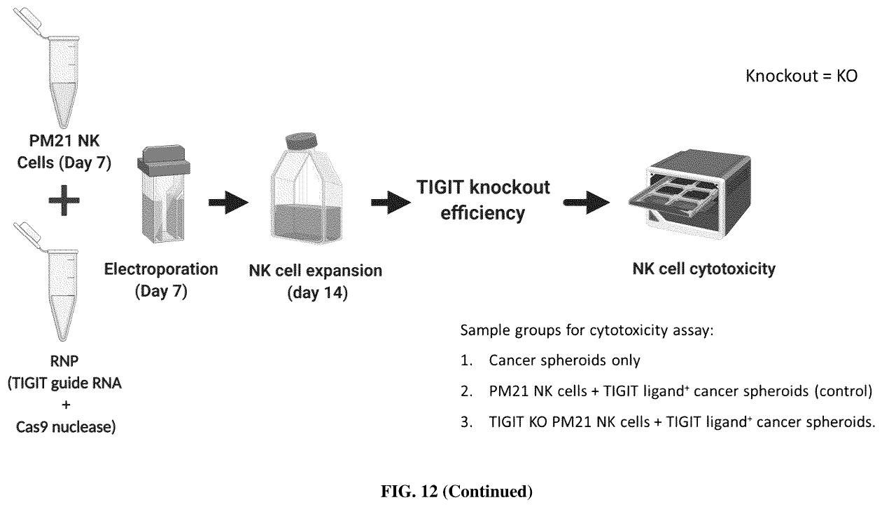

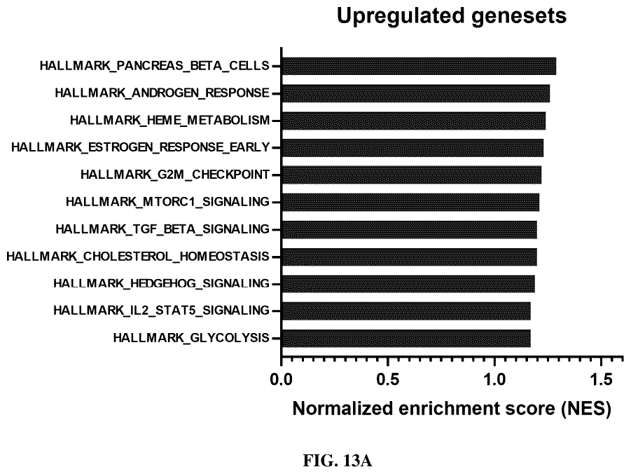

1.1. Determine the effect of TIGIT signaling on cytotoxicity and effector function of PM21 NK cells. It was observed that blockade of TIGIT improved cell cytotoxicity and the expression of IFN-7, TNF-α, and surface CD107a of NK cells against A549 spheroids (FIGS. 1D and 1F). TIGIT signaling was further blocked with anti-TIGIT antibodies during co-culture of PM21 NK cells with other TIGIT ligand+ cancer cell 3D spheroids for 7 days and then cytotoxicity was analyzed with the live imaging system (FIGS. 6 and 7). PM21 NK cells were co-cultured with cancer cell spheroids in the presence of isotype control antibodies as a negative control. After co-culture, NK cells were restimulated with PVR+K562 cells, and the expression of IFN-7, TNF-α, and surface CD107a were analyzed to determine their effector function. PM21 NK cells that were not co-cultured were used as a positive control. A schematic of these experiments is shown in FIG. 12.

PM21 expanded NK cells have higher cytotoxicity in the presence of TIGIT antibodies. PM21 NK cells express less IFN-7, TNF-α, and surface CD107a upon co-culture with other cancer spheroids, while blockade of TIGIT signaling can restore their expression (FIGS. 1D-1F).

1.2. Identify the TIGIT signaling regulated pathways and genes in PM21 NK cells by transcriptome analysis. Inhibitory receptor signaling may promote cellular reprogramming in NK cells to prevent anti-tumor activity and promote exhaustion. To understand which cellular pathways and genes are regulated by TIGIT signaling in NK cells, PM21 NK cells were co-cultured with A549 cancer spheroids in the presence of anti-TIGIT and isotype antibodies. After incubation with cancer cells, NK cells were selected with a CD56 positive selection kit and RNA was extracted to perform RNA-seq. After assembly and normalization, significantly differentially expressed genes can be analyzed with a cut-off P value <0.05. dysregulated pathways, biological processes, and functions are determined by performing KEGG pathways and gene ontology (GO) analysis.

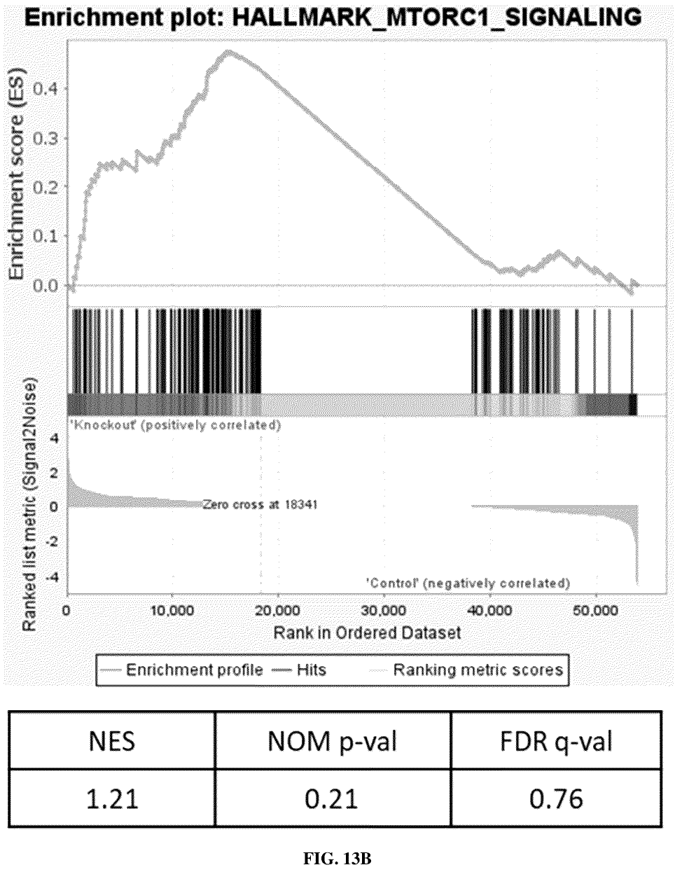

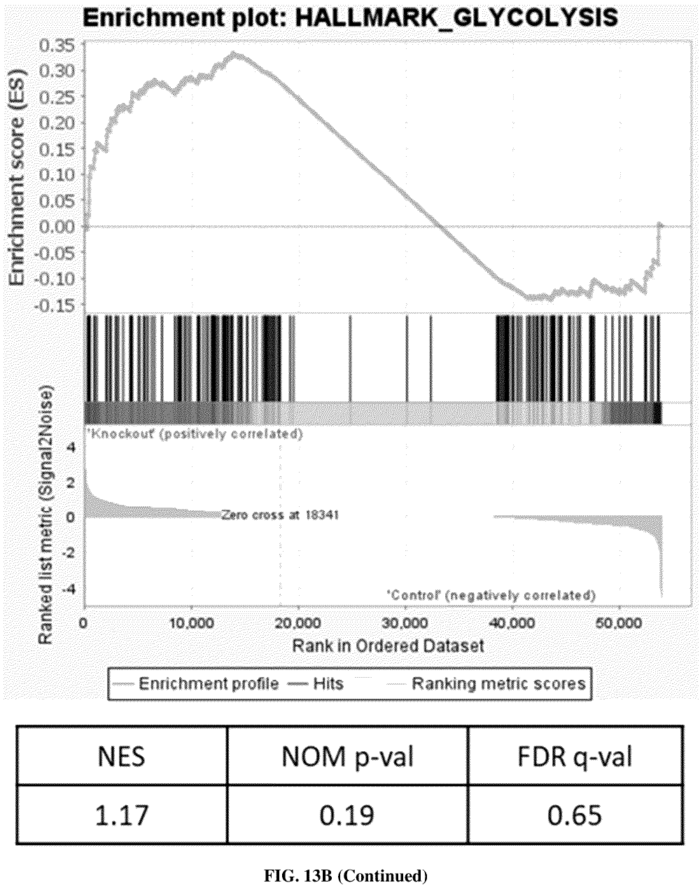

Additionally, gene set enrichment analysis (GSEA) was conducted to understand functionally related genes that are involved in the exhaustion process (FIGS. 13A-B and 14). RNA-seq expression analysis results are further validated by qRT-PCR, western blot, and flow cytometry.

In these studies, it was observed that the specific pathways and genes regulated by TIGIT signaling, especially those related to the immune system, metabolism, and epigenetic regulation, can be dysregulated after the co-culture of NK cells and cancer spheroids. These findings provide significant insight related to the exhaustion process, which can be exploited further to improve the anti-tumor activity of PM21 NK cells.

1.3. Examine the effect of TIGIT signaling on metabolic properties of PM21 NK cells. To understand whether TIGIT signaling regulates NK cell metabolism, cell energy phenotyping is conducted by simultaneously measuring mitochondrial respiration and glycolysis potential. PM21 NK cells can be positively selected from co-culture with A549 cancer spheroids in the presence of anti-TIGIT (experimental) and isotype control (negative control) antibodies and metabolic phenotypes are analyzed using the Seahorse XF analyzer.

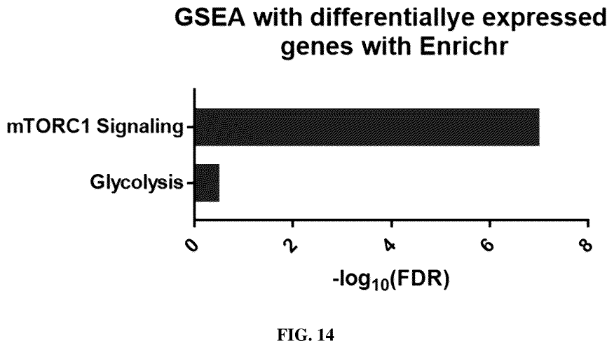

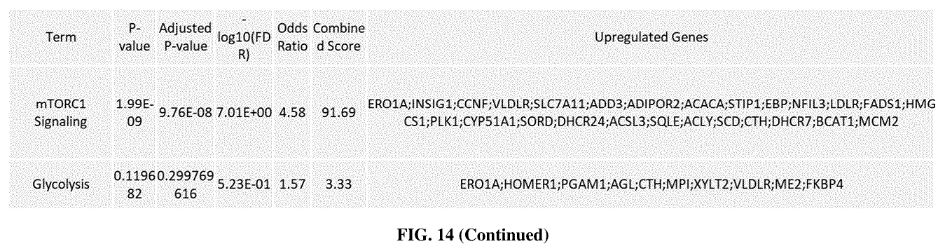

GSEA gene enrichment analysis was conducted for genes related to glycolysis, mitochondrial respiration, and pathways related to metabolism including mTOR and PI3K signaling with dysregulated genes that was determined (FIGS. 13 and 14).

These studies generate valuable insights into whether TIGIT signaling regulates NK cell metabolism. Additionally, the study determines metabolic pathways and genes involved in NK cell exhaustion that are regulated through TIGIT signaling.

### Example 3. Knockout of the Inhibitory Receptor TIGIT Enhances Anti-Tumor Response of Ex Vivo Expanded NK Cells

NK cells are an important immune cell population that are crucial for the success of many immunotherapies due to their role in both the innate response of the immune system and in priming an adaptive immune response. Recently, much focus has been on generating highly cytotoxic NK cells for use in adoptive cell therapy and combinatorial immune-oncology therapies. The robust cytotoxicity against cancer cells and NK cell activation relies on fine tuning of activating and inhibitory signals. NK cell inhibitory receptors are often upregulated upon stimulation and activation and can be a marker for exhaustion. One of the major inhibitory receptors on both NK and T cells, TIGIT, is highly expressed in ex vivo expanded NK cells. This study investigates if knockout of TIGIT in ex vivo expanded NK cells enhances their anti-tumor activity.

CRISPR was used to make a targeted TIGIT knockout out (KO) in ex vivo expanded NK cells. TIGIT KO NK cells were then compared to wild type NK cells to determine any changes in phenotypic markers. IFNγ, TNFα, and the degranulation marker CD107a expression were then analyzed after co-culture with cancer cells. Finally, cytotoxicity of TIGIT KO NK cells was compared to wild type NK cells against multiple different cancer cell spheroids using a kinetic live-cell imaging assay. Multiple NK cell:target cell ratios were analyzed over time to determine killing half-time and maximum killing. Data were also fit to a dose-response curves to determine cytotoxicity EC₅₀ values.

To examine the effect of suppressing TIGIT expression, PM21 NK cells were electroporated with TIGIT specific guide RNA and Cas9 RNP complex to knockout TIGIT. The initial CRISPR-based knockout efficiency was more than 75% (FIG. 1G). To examine the effect of TIGIT knockout on the cytotoxicity of PM21 NK cells, TIGIT KO NK cells were co-cultured with A549 spheroids. Similar to TIGIT blockade with anti-TIGIT antibodies, TIGIT knockout in PM21 NK cells increased the cytotoxicity against A549 cell spheroids (FIG. 1H). These findings strongly indicate that suppressing TIGIT expression improves anti-tumor activity of PM21 NK cells.

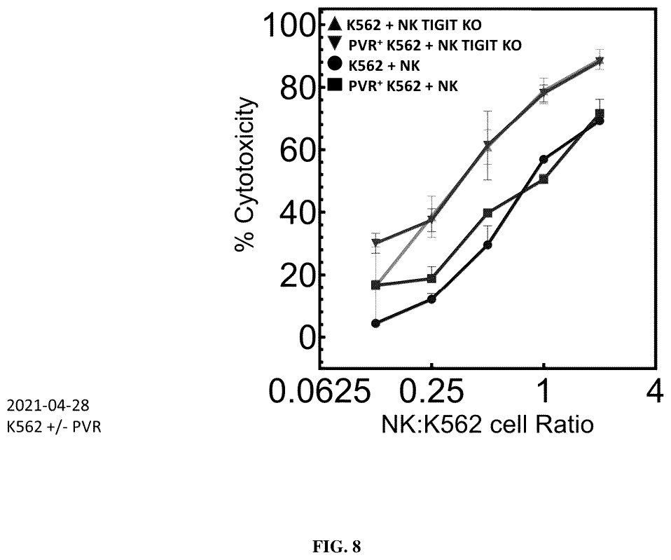

1) Modify TIGIT signaling of PM21 NK cells to enhance anti-tumor activity. In this study, PM21 expanded TIGIT knockout NK (TIGIT KO PM21 NK) cells were developed and expanded (FIG. 1G) and their anti-tumor response against cancer cells was evaluated (FIG. 1H and FIG. 8). These experiments showed that TIGIT KO PM21 NK cells exhibited higher cytotoxicity against TIGIT ligand+A549 lung cancer cell spheroids (FIG. 1H).

A CRISPR-based knockout method was optimized and established for achieving better knockout efficiency, viability, and expansion of TIGIT KO PM21 NK cells. For further studies, the cytotoxicity of TIGIT KO PM21 NK cells against multiple TIGIT ligand+ cancer cell spheroids was determined. To understand the effect on TIGIT KO PM21 NK cells, the functional and phenotypic properties of TIGIT KO PM21 NK cells were examined with flow cytometry-based protein expression analysis and RNA-seq based transcriptome analysis after expansion and after co-culture with cancer spheroids in vitro. To determine the effect of TIGIT knockout on PM21 NK cell metabolism, the metabolic phenotypes of TIGIT KO PM21 NK cells were examined. Finally, the effect of TIGIT knockout on NK cell persistence and anti-tumor activity is determined in NSG mice.1.1. Develop and characterize TIGIT KO PM21 NK cells. To optimize the TIGIT knockout efficacy, PM21 expanded NK cells were electroporated with multiple guide RNAs and Cas9 nuclease. After achieving desired (>90%) knockout efficacy, the best guide RNA for our method was selected. Off-target effects can be analyzed with the genome-wide sequencing (guide-seq) method. TIGIT KO PM21 NK cells and regular PM21 NK cells (negative control) were co-cultured with TIGIT ligand+ cancer cell spheroids and analyzed with a live imaging system to determine the effect of TIGIT knockout on PM21 NK cell cytotoxicity. After cytotoxicity, NK cells were restimulated with PVR+K562 cells to examine the expression of IFNγ, TNFα, and surface CD107a to understand the functional properties of TIGIT KO NK cells.

This method generated TIGIT KO PM21 NK cells with high knockout efficacy (>90%) and high viability. Furthermore, TIGIT KO PM21 NK cells were shown to have significantly superior cytotoxicity and express more IFNγ, TNFα compared to regular PM21 NK cells against TIGIT ligand+ cancer cells compared to parental PM21 NK cells.

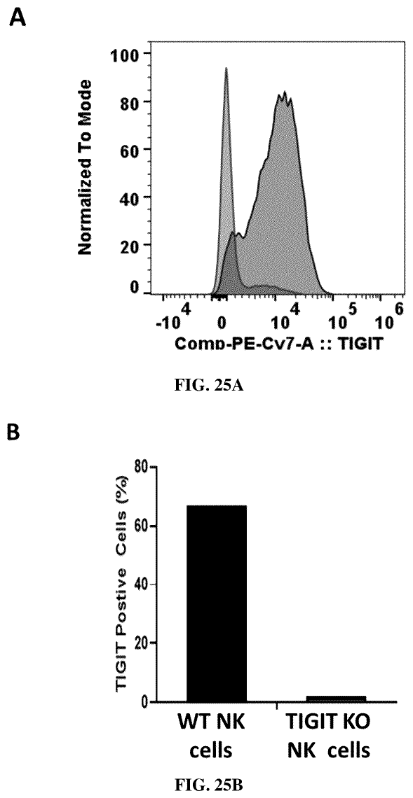

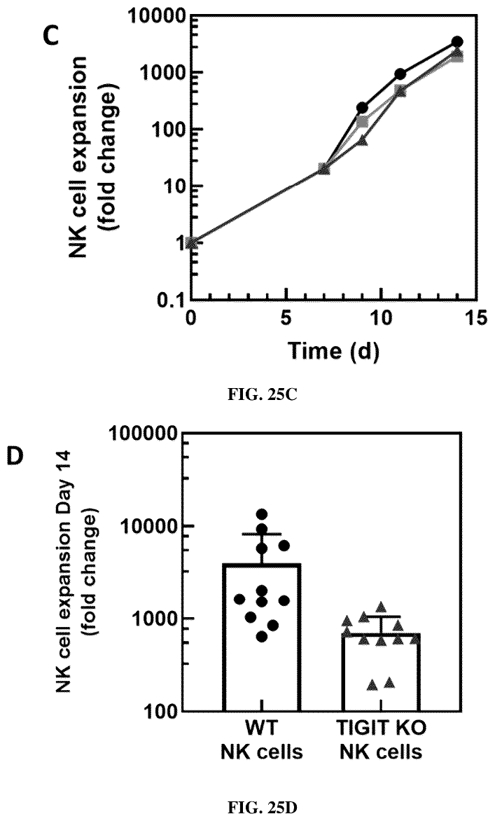

NK cells were electroporated with TIGIT specific CRISPR/Cas9 ribonucleoprotein (RNP) complex (TIGIT KO PM21-NK cells) or without RNP (no RNP control PM21-NK cells) on day 7 and expanded with PM21 particles (PM21-NK cells) for total of 2 weeks. See FIG. 12. Representative histogram showing wild-type PM21-NK cells (WT PM21-NK cells) (grey) and TIGIT KO PM21-NK cells (red) (FIG. 25A). TIGIT knockout (KO) efficiency was more than 90% in TIGIT KO PM21-NK cells compared to WT PM21-NK cells (FIG. 25B). Representative expansion curve indicates significant expansion of TIGIT KO PM21-NK cells comparable to WT PM21-NK cells (black) and no RNP control PM21-NK cells (grey) (FIG. 25C). Summary graph represents the expansion of WT PM21-NK cells (mean 3,999-fold) and TIGIT KO PM21-NK cells (mean 702.5-fold) (N=11 donors) (FIG. 25D). Data are presented as scatter plots or bar graphs with error bars representing standard deviation.

1.2. Effect of TIGIT deletion on phenotypic, metabolic, and transcriptomic properties of NK cells. To understand the overall effect of TIGIT knockout on PM21 NK cells, phenotypic, metabolic, and transcriptomic profiles of TIGIT KO and parental PM21 NK cells were determined after expansion and after co-culture with cancer spheroids. NK cells were collected after expansion and after co-culturing with cancer spheroids. To study phenotypic properties, NK cells were stained with antibodies against NK cell markers and analyzed with flow cytometry. To examine metabolic properties, collected NK cells were analyzed to determine glycolysis and mitochondrial respiration with a Seahorse analyzer.

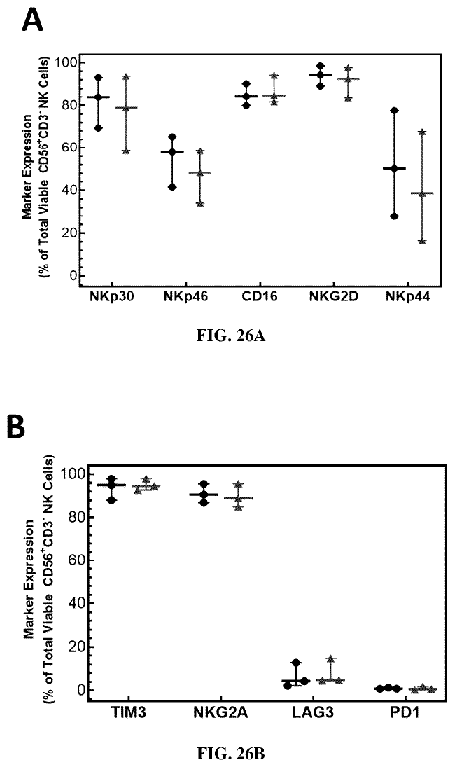

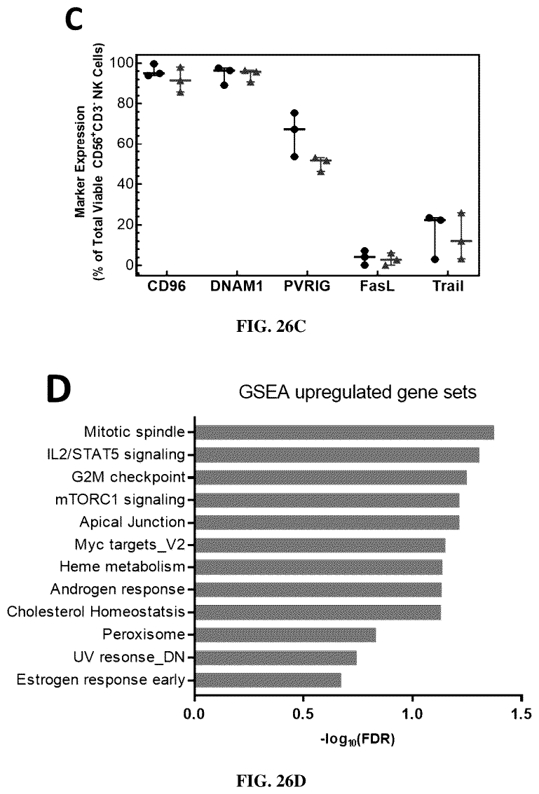

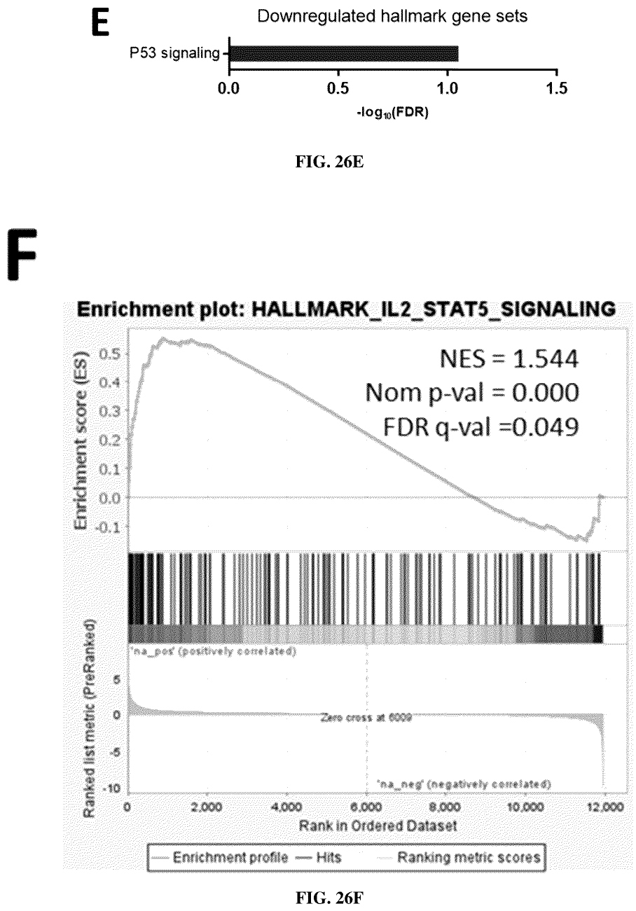

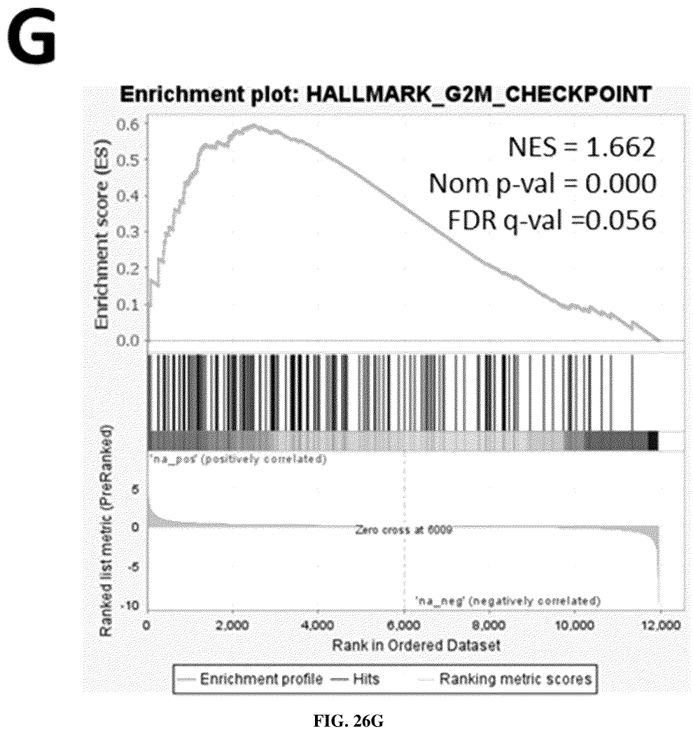

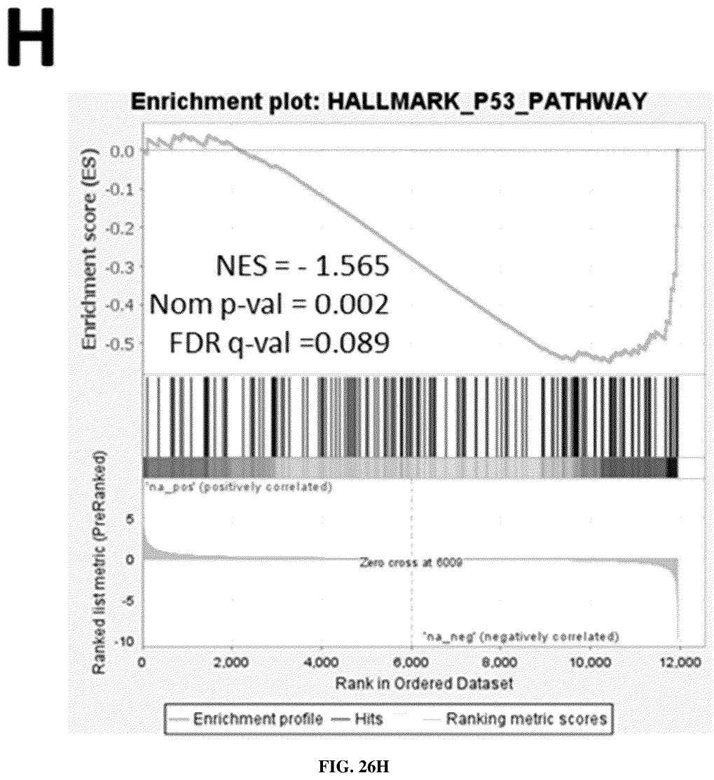

WT PM21-NK cells and TIGIT KO PM21-NK cells were expanded for 12 days and expression of the activating, and inhibitory receptors on WT PM21-NK cells (black) and TIGIT KO PM21-NK cells (red) were determined by flow cytometry. TIGIT knockout did not significantly alter the expression of activating receptors (FIG. 26A), inhibitory receptors (FIG. 26B) and CD96-DNAM1 axis, FasL and Trail (FIG. 26C) (N=3 donors, each average of duplicates). Expanded WT and TIGIT KO PM21-NK cells were used for RNA extraction and bulk RNA sequencing was conducted to determine dysregulated gene sets. Gene set enrichment (GSEA) analysis indicates that TIGIT knockout upregulated multiple hallmark gene sets including IL2/STAT5 signaling and G2M checkpoint related gene sets (FIG. 26D), while downregulated P53 pathway (FIG. 26E). Enrichment plots for IL2/STAT5 signaling (FIG. 26F), G2M checkpoint (FIG. 26G) and P53 pathway (FIG. 26H). Data are presented as scatter plots or bar graphs with error bars representing standard deviation. Statistical significance was determined by multiple unpaired t-tests. P values are shown as * if p<0.05, ** if p<0.01, *** if p<0.001, **** if p<0.0001

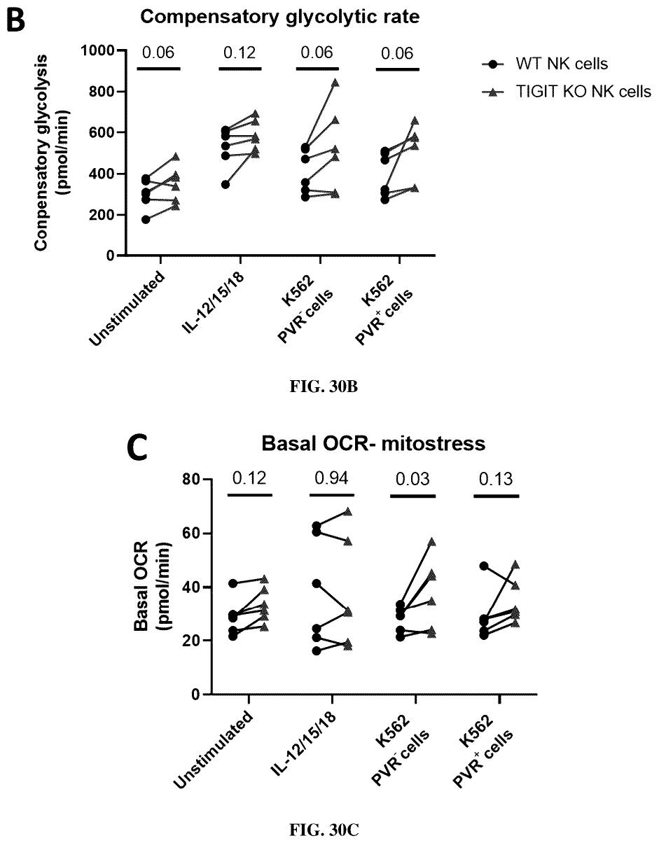

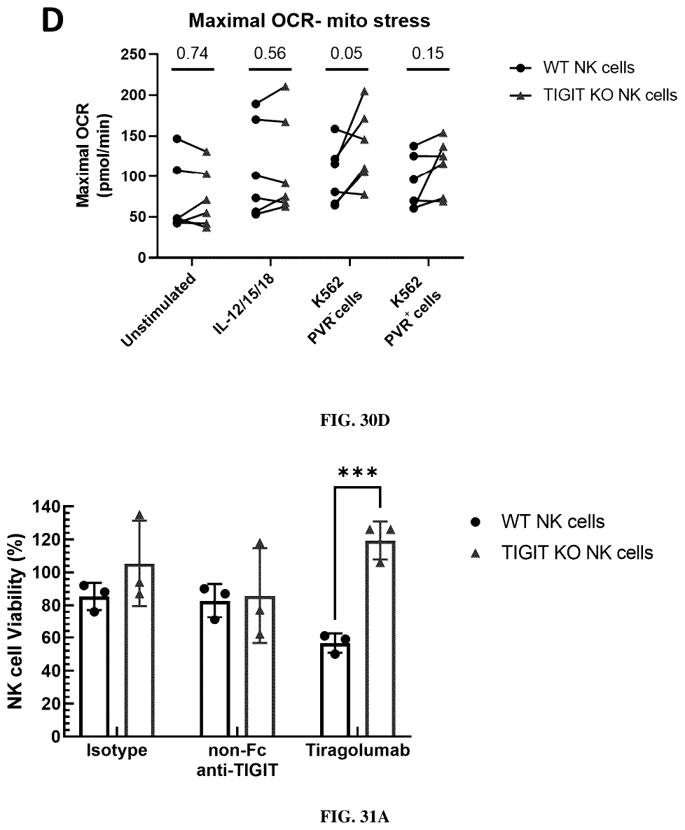

WT PM21-NK cells and TIGIT KO PM21-NK cells were stimulated with or without IL12 (10 ng/ml), IL15 (100 ng/ml) and IL18 (50 ng/ml), with PVR negative K562 cells (K562_PVR⁻ cells) or PVR positive K562 cells (K562_PVR+ cells) for 24 hours and NK cells were used for glycolysis rate and mitochondrial stress test with seahorse system. K562_PVR⁻ and K562_PVR+ cell stimulation significantly increased both basal and compensatory glycolytic rate in TIGIT KO PM21-NK cells (solid red triangle) compared to WT PM21-NK cells (black circle) (N=6 donors) (FIGS. 30A-30B), while only K562_PVR⁻ cells increased basal oxygen consumption rate (OCR) in TIGIT KO PM21-NK cells (N=4 donors) (FIGS. 30C-30D). Data are presented as scatter plots with donor-pair lines. Statistical significance was determined by multiple paired t-tests. P values are shown as * if p<0.05, ** if p<0.01, *** if p<0.001, **** if p<0.0001.

RNA was extracted from collected NK cells and was used to do transcriptome profiling of parental and TIGIT KO PM21 NK cells with total RNA seq. Raw data were normalized and significantly differentially expressed genes with a cut-off P value <0.05.

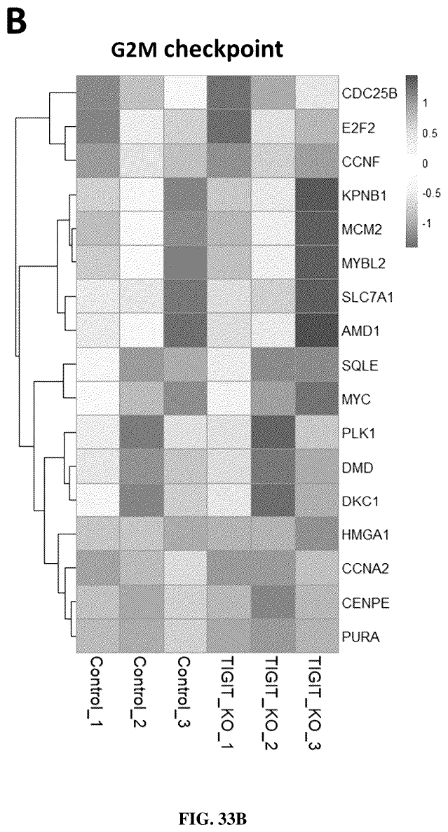

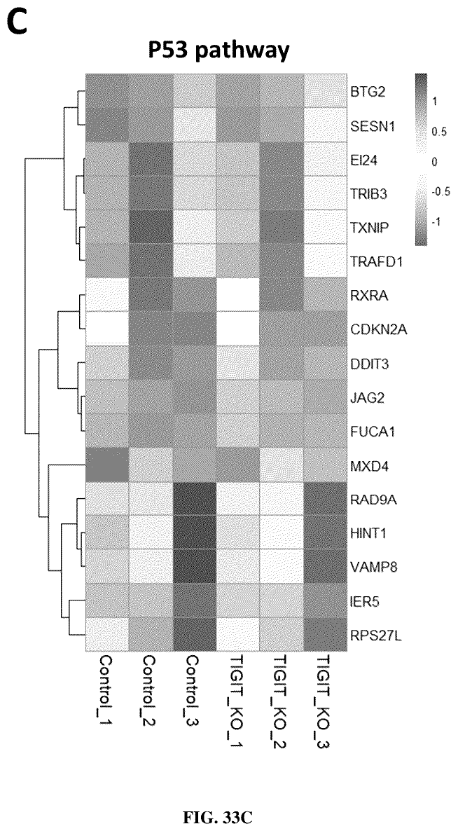

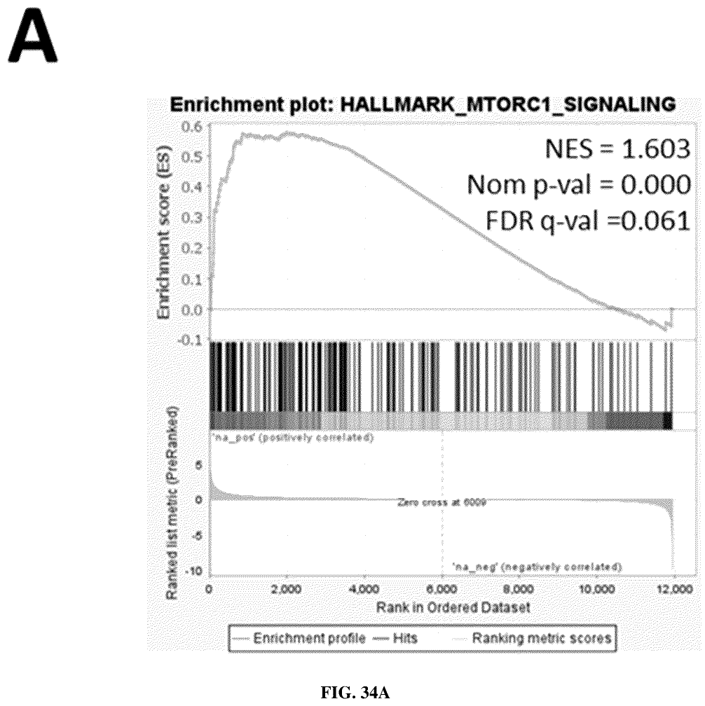

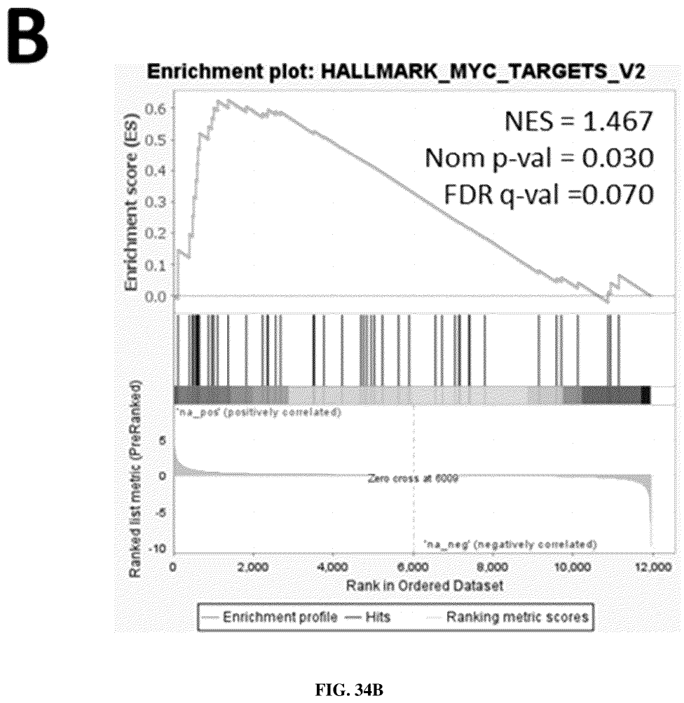

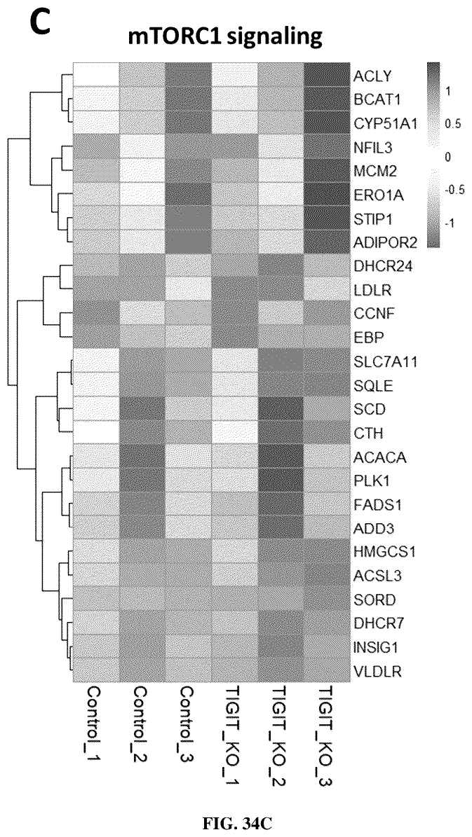

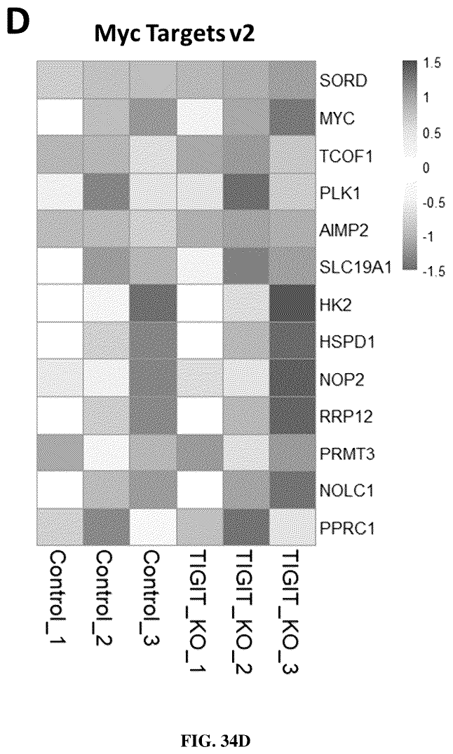

KEGG pathway analysis, GO analysis and GSEA gene enrichment analysis were conducted to determine TIGIT regulated pathways, biological processes, and functions of related gene sets. For all these experiments, parental PM21 NK cells were used as control. Gene set enrichment analysis of RNA-seq data showed that TIGIT KO NK cells upregulated hallmark gene sets for mTORC1 signaling and glycolysis, indicating increased growth and metabolism in the TIGIT KO NK cells. FIG. 33 shows heatmap of representative up and downregulated gene sets upon TIGIT KO in NK cells, and FIGS. 34A-34D show metabolic analysis of TIGIT KO NK cells.

TIGIT knockout NK cells can also prevent dysregulation of important pathways and genes involved in inhibiting anti-tumor activity and exhibit more active phenotypes. Furthermore, metabolic profiling determines TIGIT signaling regulated NK cell metabolism.

CRISPR was used to efficiently knockout TIGIT in ex vivo expanded NK cells and decreased expression levels to less than 5%. After co-culture with Raji cells expressing the TIGIT ligand PVR, TIGIT KO NK cells showed increased expression of IFNγ, TNFα and CD107a. TIGIT KO NK cells showed improved killing compared to wild type NK cells. TIGIT KO cells killed more target cells faster with significant decreases in half-killing time and EC₅₀ cytotoxicity values in 3D spheroid models of six different cancer cell lines. When NK cell:target cell ratios were low the maximum cytotoxicity was also higher in TIGIT KO cells.

1.3 Effect of TIGIT deletion on cytokine expression, degranulation and cytotoxicity of NK cells. Deletion of the TIGIT gene in ex vivo expanded NK cells resulted in NK cells with increased cytokine expression, degranulation, and cytotoxicity. These TIGIT knockout NK cells with improved antitumor activity provide a universal effector population with the potential for enhanced therapeutic efficacy.

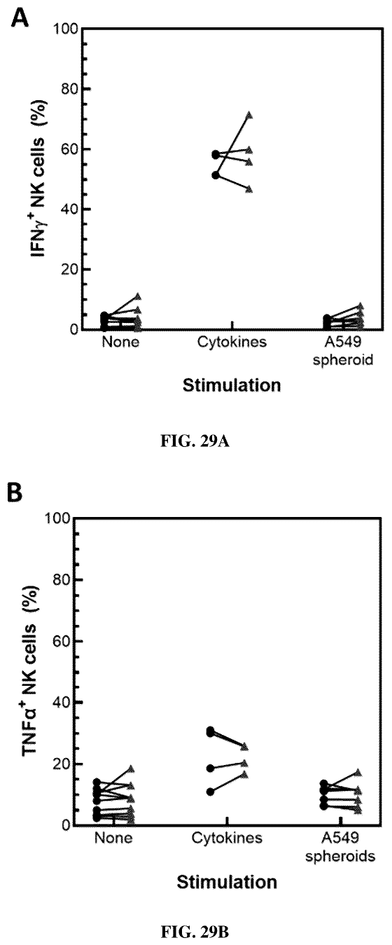

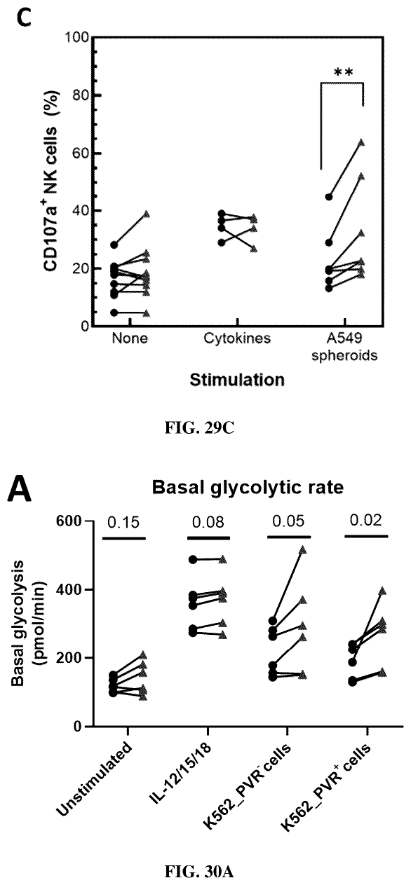

Ex vivo expanded WT PM21-NK cells and TIGIT KO PM21-NK cells were left untreated, co-cultured with A549 spheroids for 48 h or stimulated 4 hours with IL12 (10 ng/ml), IL15 (100 ng/ml) and IL18 (50 ng/ml). Golgi stop and Brefeldin A were added 4 hours before analysis and NK cell expression of IFNγ, TNFα and surface CD107a by flow cytometry. TIGIT knockout significantly increased surface CD107a expression on TIGIT KO PM21-NK cells (solid red triangle) against A549 spheroids (FIG. 29C) but did not increase IFNγ (FIG. 29A) and TNFα (FIG. 29B) compared to WT PM21-NK cells (solid black circle) (N=4-8 donors, each average of duplicates). Data are presented as scatter plots with donor-pair lines. Statistical significance was determined by multiple paired t-tests. P values are shown as * if p<0.05, ** if p<0.01, *** if p<0.001, **** if p<0.0001.

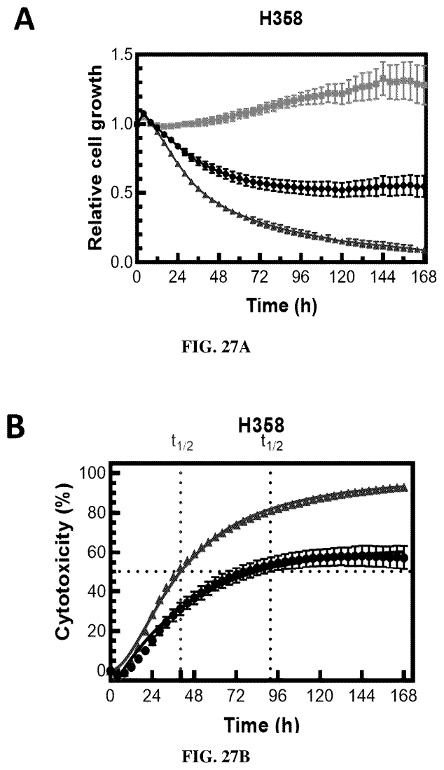

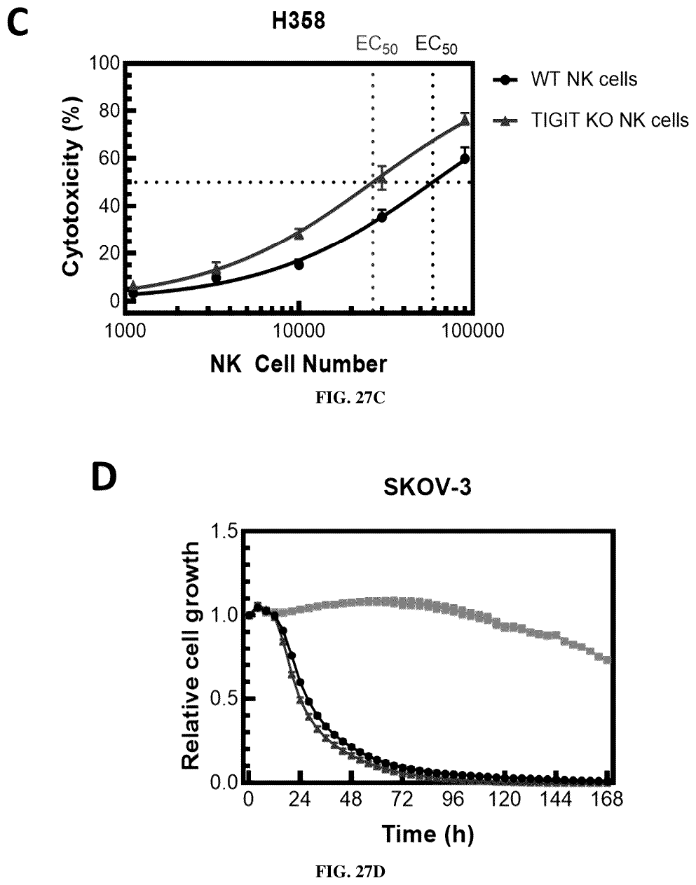

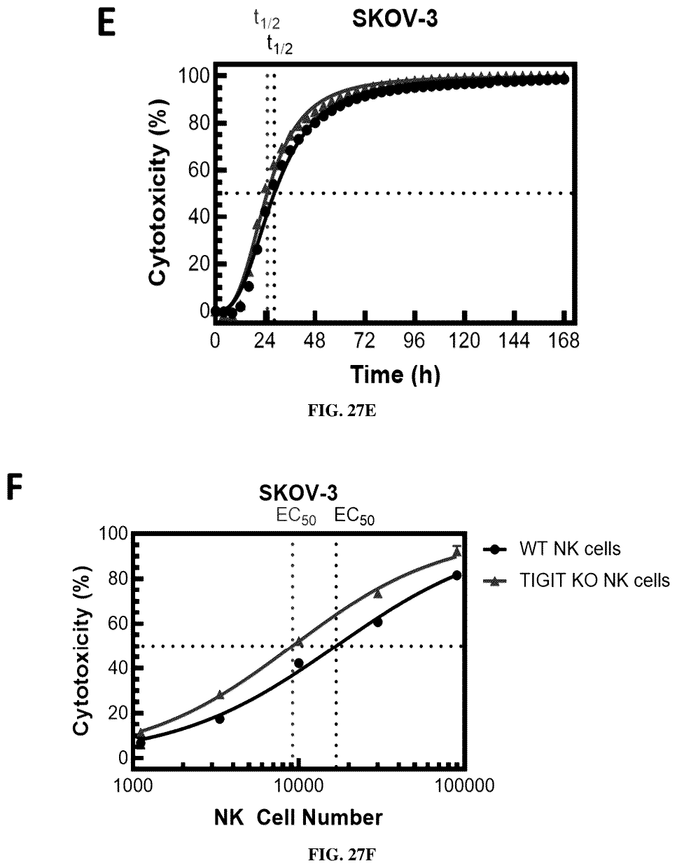

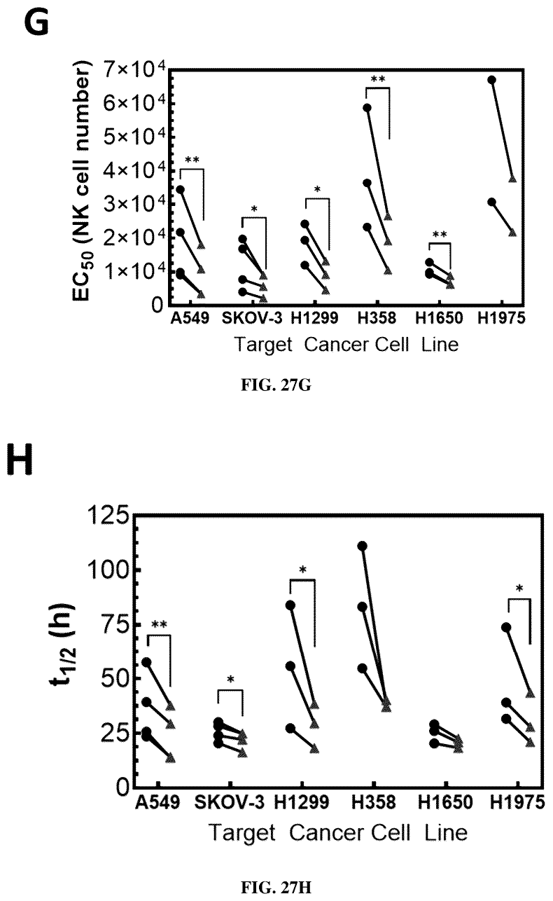

Expanded WT PM21-NK cells and TIGIT KO PM21-NK cells were co-cultured with ovarian SKOV3 and lung A549, NCI-H358, NCI-H1299, NCI-H1650 or NCI-H1299 cancer cell spheroids for 7 days. NK cell cytotoxicity was determined by kinetic live-cell imaging. Representative raw data from one experiment is shown for NCI-H358 and SKOV-3 relative expansion alone (grey squares) or in the presence of WT PM21-NK cells (black) or TIGIT KO PM21-NK cells (red) with 10000 NK cells (NCI-H358) and 3333 NK cells (SKOV3) (FIGS. 27A and 27D). Representative plots for NK cell cytotoxicity (FIGS. 27B and 27E) and EC₅₀ (FIGS. 27C and 27F) are also shown. Summary plots of NK cell cytotoxicity shows that TIGIT KO-PM21 NK cells significantly increases EC₅₀ (FIG. 27G), t_{1/2} (FIG. 27H) and cytotoxicity maximum (FIG. 27I) compared to WT PM21-NK cells (N=3 donors, each average of triplicates). Data are presented as scatter plots with donor-pair lines or as mean with error bars representing standard deviation. Statistical significances were determined by multiple unpaired or paired t-tests. P values are shown as * if p<0.05, ** if p<0.01, *** if p<0.001, **** if p<0.0001.

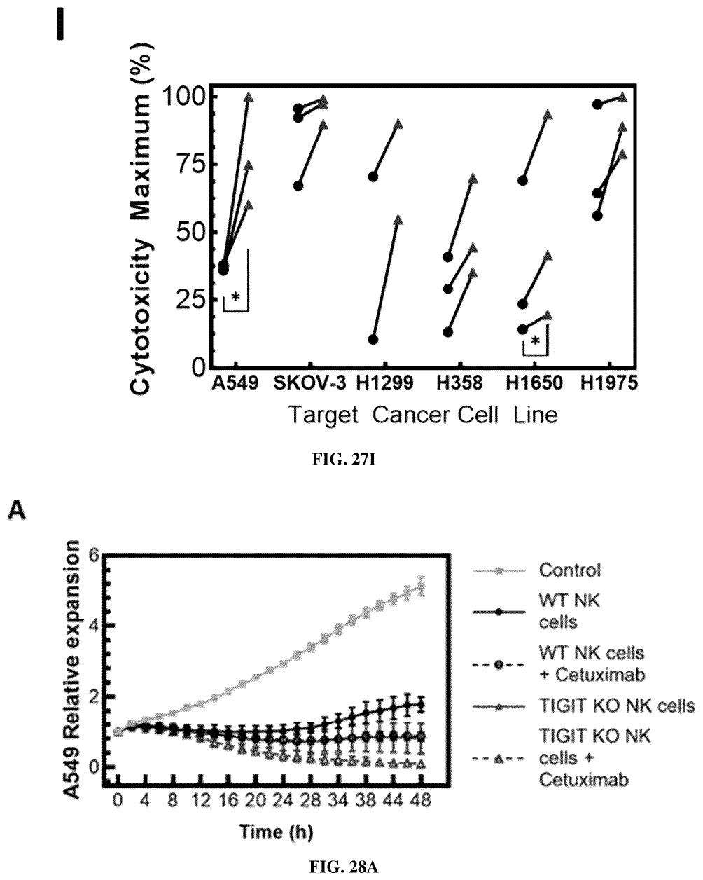

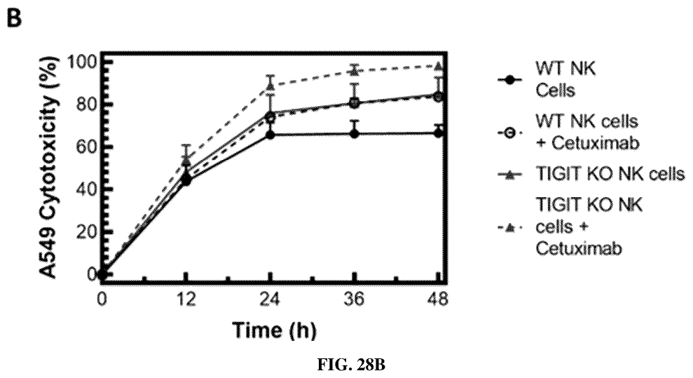

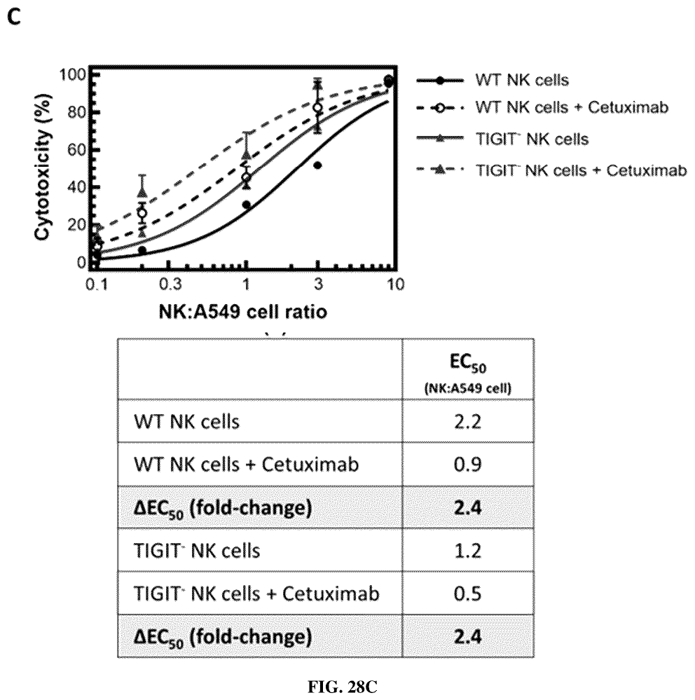

Ex vivo expanded WT PM21-NK cells and TIGIT KO PM21-NK cells were co-cultured with A549 cells (2D) for 48 hours with and without Cetuximab. NK cell cytotoxicity was determined by kinetic live-cell imaging. Representative graph shows relative expansion of A549 cells alone (grey square), WT PM21-NK cells (solid black circle), WT PM21-NK cells with cetuximab (black circle), TIGIT KO PM21-NK cells (solid red triangle) and TIGIT KO PM21-NK cells with cetuximab (red triangle) (FIG. 28A). representative cytotoxicity plots (FIG. 28B) demonstrate that TIGIT KO (red) killed better A549 cells as compared to WT NK cells (black) and the addition of cetuximab further improved NK cell cytotoxicity of both WT and TIGIT KO NK cells (dotted lines). Summary plot and table of NK cell cytotoxicity against A549 cells at multiple NK:T ratio showed cetuximab further improves cytotoxicity of TIGIT KO PM21-NK cells with a EC₅₀ fold change 2.4 (N=2 donors, each average of duplicate) (FIG. 28C).

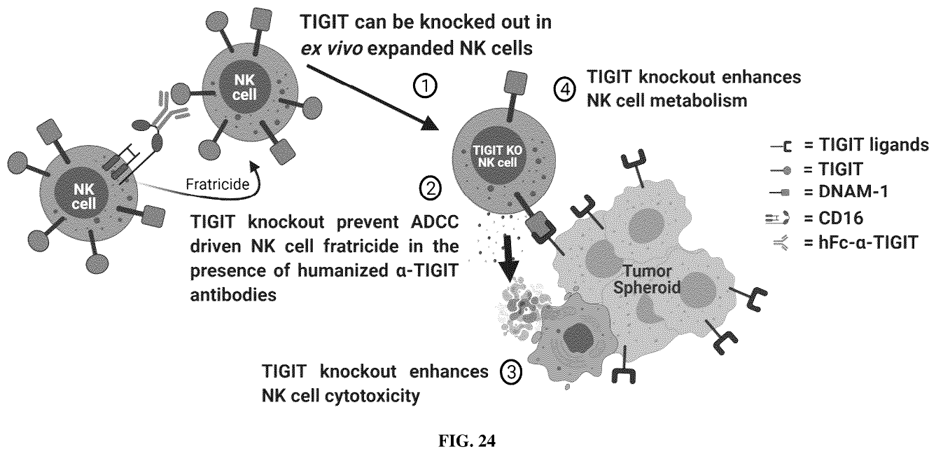

As summarized in the schematic of FIG. 24, TIGIT KO NK cells have enhanced proliferation and cytotoxicity, including ADCC, compared to WT NK cells. 1) TIGIT KO NK cells have increased TNFα production upon stimulation with PVR⁺ tumor cells compared to WT NK cells. 2) Tumor spheroid-exposed TIGIT KO NK cells have increased degranulation compared to WT PM21-NK cells. 3) Upregulation of mTOR signaling, glycolysis, and IL2-STAT5 signaling was observed in TIGIT KO PM21-NK cells by RNA-seq analysis. 4) Enhanced metabolic fitness was observed compared WT PM21-NK cells upon to stimulation with PVR⁺ tumor cells. 5) TIGT KO prevents ADCC driven NK cell fratricide and prevents decrease in cytotoxicity when combined with Fc-competent α-TIGIT antibody.

### Example 4. Examine the Persistence and Anti-Tumor Activity of TIGIT KO PM21 NK Cells In Vivo

Cancer cells are seeded intraperitoneally in NSG mice and incubated for 7 days. After that, parental and TIGIT KO PM21 NK cells are injected intraperitoneally. IL-2 is injected 2× each week to support NK cells. Mice are monitored over time to examine tumor growth and survival. A subgroup of mice are sacrificed after 14 days of NK cell injection. NK cells are collected to determine NK cell persistence and phenotypes. Additionally, NK cells re restimulated with K562_PVR+ cells and the effector function of TIGIT KO and parental PM21 NK cells re analyzed and compared. Parental PM21 NK cells are used as a control in these experiments.

TIGIT KO PM21 NK cells have better persistence and retain more robust antitumor activity in vivo compared to parental PM21 NK cells. Furthermore, TIGIT KO PM21 NK cells reduce tumor growth and increase tumor-bearing mice survival compared to parental PM21 NK cells.

Previous clinical studies showed that TIGIT blockade alone was not able to reduce tumor growth and survival significantly. Other studies showed that TIGIT blockade increased the anti-tumor immunity of NK cells significantly. However, anti-TIGIT antibodies in combination with anti-PD-1/PD-L1 or anti-TIM3 significantly improved survival and further reduced tumor growth. A combination therapy of TIGIT KO PM21 NK cells with anti-PD-1/PD-L1 or anti-TIM3 can be utilized.

### Example 5. Evaluate the Therapeutic Efficacy of PM21 NK Cells in Combination with Blockade of TIGIT In Vivo

This example examines whether PM21 NK cells can exhibit better anti-tumor activity in presence of anti-TIGIT, TIGIT ligand+ cancer cells are injected intraperitoneally in NSG mice. PM21 NK cells along with anti-TIGIT (experimental) or isotype control (positive control) are administered to treat the engrafted mice. IL-2 is injected 2× each week to support the NK cells. The tumor growth and survival is analyzed over time. A subgroup of mice are sacrificed after 14 days, and NK cells are collected via peritoneal wash. Recovered NK cells are analyzed in order to determine persistence and phenotypic properties. NK cells can exhibit higher anti-tumor activity in combination with anti-TIGIT, which improves tumor control, and thus the overall survival compared to expanded NK cells with isotype or anti-TIGIT alone injected mice groups. Furthermore, the TIGIT blockade increases the persistence of NK cells.

In in vivo studies, both anti-TIGIT and anti-PD-L1 antibodies are injected along with PM21 NK cells. Previous studies also showed that Treg cells play a role in TIGIT signaling and anti-TIGIT efficacy depends on NK cell-based deletion of Treg cells. Treg cells can be injected with PM21 NK cells and anti-TIGIT antibodies and the effect of TIGIT signaling on PM21 NK cells is examined in presence of Treg cells in vivo.

### Example 6. TIGIT Blockade Improves Anti-Tumor Activity of Ex Vivo Expanded NK Cells by Preventing PVR⁻Mediated Exhaustion in Models of Lung Cancer

Ex vivo expanded Natural Killer (NK) cells have gained interest as an adoptive immunotherapy because of their robust response against cancers. NK cells express activating and inhibitory receptors that regulate their activities. The inhibitory receptor TIGIT is often upregulated on NK cells in cancers, inhibits NK cell activity and promotes NK cell exhaustion. Most of the understanding on TIGIT's role in NK cell function comes from murine models while mechanistic in vitro studies using human NK cells are limited. In this study, the effect of TIGIT blockade on anti-tumor activities of human ex vivo expanded NK cells was evaluated.

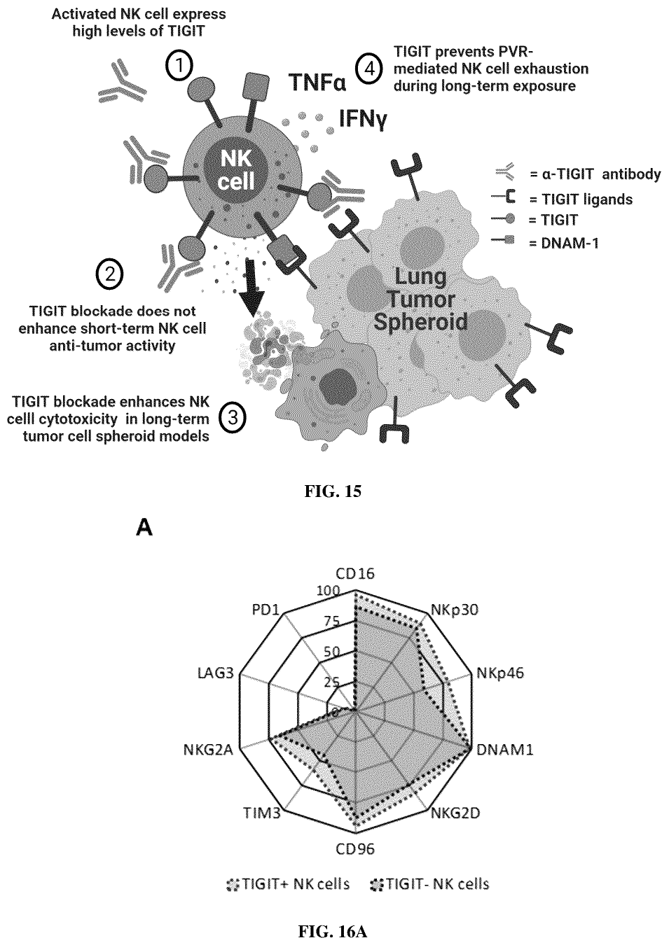

TIGIT expression was determined on cytokine activated and PM21-particle expanded human NK cells (PM21-NK cells) by qRT-PCR and flow cytometry. NK cell cytotoxicity against 2D and 3D models of multiple lung cancer cell lines was assessed by kinetic live imaging-based assays. To assess NK cell exhaustion, PVR+ or PVR⁻K562 cells were used to restimulate A549 spheroid-exposed PM21-NK cells and surface expression of CD107a and production of IFNγ and TNFα were measured. Without intending to be limited by theory, a potential mechanism of TIGIT blockade preventing PVR⁻mediated exhaustion is illustrated in FIG. 15. RNA-seq analysis was performed on PM21-NK cells selected after co-culture with A549 spheroids with or without TIGIT blockade.

TIGIT is highly expressed on ex vivo expanded and activated human NK cells. TIGIT blockade significantly improves cytotoxicity of PM21-NK cells against lung cancer spheroids and restores effector functions against PVR positive cancer cells after long-term exposure to cancer cell spheroids.

In this example, the effect of TIGIT blockade on the anti-tumor activities of PM21-particle expanded-NK cells (PM21-NK cells) was investigated in lung cancer model. Ex vivo expansion and/or activation upregulated TIGIT on NK cells. While TIGIT blockade did not increase anti-tumor activities of PM21-NK cells against lung cancer cells in monolayers, TIGIT blockade increased PM21-NK cell cytotoxicity against 3D lung cancer spheroids and prevented PVR⁻mediated exhaustion after long term exposure to tumor spheroids. TIGIT blockade upregulated multiple gene sets related to NK cell anti-tumor responses including inflammatory response-related genes, TNFα signaling via NFkB, and IFNγ response-related gene sets. This study demonstrated that TIGIT blockade prevents PVR⁻induced decrease of NK cell function and increases overall anti-tumor response of ex vivo expanded primary human NK cells.

### Methods

Cell culture. Buffy coats (Leukocyte Source) from de-identified healthy donors were used as a source of NK cells and were purchased from a local blood bank (OneBlood). Peripheral blood mononuclear cells (PBMC) were separated by density gradient (Ficoll-Paque Plus solution; GE Healthcare, Chicago, IL, USA) and cryopreserved for further use. NK cells were expanded with PM21-particles as described previously (19,20,47). Briefly, T cell-depleted PBMC (EasySep CD3 positive selection kit; StemCell Technologies, Vancouver, Canada) were stimulated with 200 μg/mL PM21-particles and cultured for 2 to 3 weeks in SCGM media (CellGenix GmbH, Freiburg im Breisgau, Germany) and RPMI media with 100 U/mL IL-2 (PeproTech, Cranbury, NJ, USA). For activated NK cells, T cell depleted PBMCs were stimulated overnight with IL-2 (1000 U/ml) or IL-12 (10 μg/ml)+IL-15 (100 μg/ml)+IL-18 (50 μg/ml). Cancer cell lines K562, A549, NCI-H358, and NCI-H1975 cells (ATCC) and NCI-H1299 (a generous gift from Dr. Griffith Parks, UCF) were maintained in RPMI media with 10% FBS, 1% antibiotic/antimycotic and 2 mM Glutamax. A549-NLR, NCI-H358-NLR, NCI-H1975-NLR and NCI-H1299-NLR cells were generated through stable transduction using commercial NucLight Red Lentivirus (Sartorius). All cell lines were positively selected via puromycin selection followed by sorting on uniform positive populations (BD FACS Aria II). All cells were maintained in a humidified atmosphere at 37° C. supplemented with 5% (vol/vol) CO2 in air. Cell lines were routinely tested for mycoplasma (E-Myco Plus Mycoplasma PCR Detection Kit, Bulldog-Bio, Inc., Portsmouth, NH, USA) and authenticated via Human STR Profiling (serviced by ATCC).

Stable cell line generation. PVR expressing K562-GFPLuc cells were generated via stable transduction using lentiviral particles generated in-house (VectorBuilder Inc., Chicago, IL, USA) containing PVR coding gene sequences and sorted for positive and negative populations with a BD FACSAria II Cell Sorter (BD Biosciences, Franklin Lakes, NJ, USA). PVR⁺ and PVR⁻ K562-GFPLuc cell lines were cryopreserved until needed.

qRT-PCR. NK cells were selected (EasySep CD56 positive selection kit StemCell Technologies, Vancouver, Canada) before and after expansion and total RNA isolated (Direct-zol RNA Microprep kit; Zymo research, Irvine, CA, USA). cDNA was synthesized (High-Capacity cDNA Reverse Transcription Kit; Applied Biosystems, Waltham, MA, USA) and the gene expression primer set for TIGIT (QuantiTect Primer Assay; Qiagen, Hilden, Germany) was used to determine RNA expression levels by qRT-PCR (Quantstudio 7 PCR system, Applied Biosystems, USA). EIF3D and RPL13A were used as control genes. The 2-AA^{CT}method (48) was used to determine relative RNA expression of the target gene.

Flow cytometry. The following antibodies were used for flow cytometry analysis: CD56-PE (clone:5.1H11), CD56-APC/Fire™750 (clone:NCAM), CD56-AF®647 (clone:5.1H11), CD3-FITC (Clone:UCHT1), TIGIT-PE/Cy7 (Clone:A15153G), CD96-PE (Clone:NK92.39), DNAM-1-FITC (Clone:TX25), PVRIG-APC (Clone:W16216D), PVR⁻PE (Clone:SKIL2), PVRL2-APC (Clone:TX31), PVRL4-AF488 (Clone:337516), NKp30-PE (Clone:P30-15), NKp46-PE/Dazzle™594 (Clone:9E2), CD16-PeCy5 (Clone:3G8), NKG2D-APC (Clone:ID11), NKp44-PE-Cy7 (Clone:P44-8), LAG-3-FITC (Clone:11C3C65), PD-1-PE-Dazzle™594 (Clone:EH12.2H7), TIM-3-PE (Clone:F38-2F2), TNFα-PE/Dazzle™594 (Clone:MAB11), IFN7-PerCP5.5 (Clone:B27), CD107a-PE (Clone:H4A3), CD3-PerCP-eF710 (Clone:OKT3), and NKG2A-APC (Clone:Z199). NK cells were stained with pre-conjugated protein-specific or the corresponding isotype control antibodies. All samples were acquired on a Cytoflex (Beckman Coulter, Brea, CA, USA) or Northern Lights 2000 Full Spectrum (Cytek, Fremont, CA, USA) flow cytometer and analyzed with CytExpert (Beckman Coulter, Brea, CA, USA; v2.4) or FlowJo software (v10.6.2).

Kinetic live-cell imaging cytotoxicity assays. Lung cancer cell lines A549-NLR, NCI-H358-NLR, NCI-H1975-NLR and NCI-H1299-NLR, stably expressing nuclear red fluorescent protein (NucLight Red; NLR) for tracking were used as target cells. For monolayer cytotoxicity assays, 6,000 cancer cells were seeded per well in a flat bottom 96-well plate the day prior to adding NK cells. For spheroid cytotoxicity assays, 5,000 cancer cells were seeded in a 96-well clear round bottom ultra-low attachment microplate (Corning, Corning, NY, USA), centrifuged at 130×g for 10 minutes and incubated for 3 days prior to form spheroids. Cancer cell monolayers or spheroids were co-cultured with NK cells at the indicated effector-to-target (E:T) ratios in the presence of Ultra-LEAF isotype or anti-TIGIT antibodies (Biolegend, San Diego, CA, USA). Monolayers were imaged for 72 h while spheroid experiments for 7 days with an IncuCyte® S3 Live-Cell Analysis System (Sartorius, Gattingen, Germany). Target tumor cell growth was tracked over time by red object count per well (ROC) in 2D assays and total red object integrated intensity (ROII) (RCU×μm²/Image) in 3D assays. Relative growth of the target cells alone or in the presence of NK cells with or without TIGIT blockade was determined by normalizing ROC or ROII to the value at time 0 (ROCₜ/ROCₜ₌₀ or ROIIₜ/ROIIₜ₌₀) when NK cells were initially added to determine normalized ROC (nROC) or normalized ROII (nROII). Cytotoxicity (%) was then determined based on the following equations.

$$2 D {Cytotoxicity}^{E : T} ( \% ) = ( 1 - ( \frac{{nROC}^{E : T}}{{nROC}^{T}} ) ) \times 100$$

$$3 D {Cytotoxicity}^{E : T} ( \% ) = ( 1 - ( \frac{{nROII}^{E : T}}{{nROII}^{T}} ) ) \times 100$$

Annexin V cytotoxicity assay. PM21-NK cells were co-cultured with target PVR+ or PVR⁻ K562-GFPLuc cells at indicated effector vs. target (E:T) ratios in the presence of Ultra-LEAF isotype or anti-TIGIT antibodies (Biolegend, San Diego, CA, USA) for 60 minutes at 37° C. in a tissue culture incubator. Cells were then centrifuged and stained with an Annexin-V-Pacific Blue antibody, incubated for 15 minutes at 4° C. and analyzed by flow cytometry. The cytotoxicity was determined based on the absolute amount of Viable Target Cells (GFP+/Annexin V−) remaining in each well with effectors (VTCE:T) and referenced to average VTC in “target alone” control wells (VTCT ctrl)

$$Cytotoxicity {}^{∧} ( E : T ) ( \% ) = 1 / ( ( VTC {}^{∧} ( E : T ) / ( Avg VTC {}^{∧} Tctrl ) ) ) \times 100$$

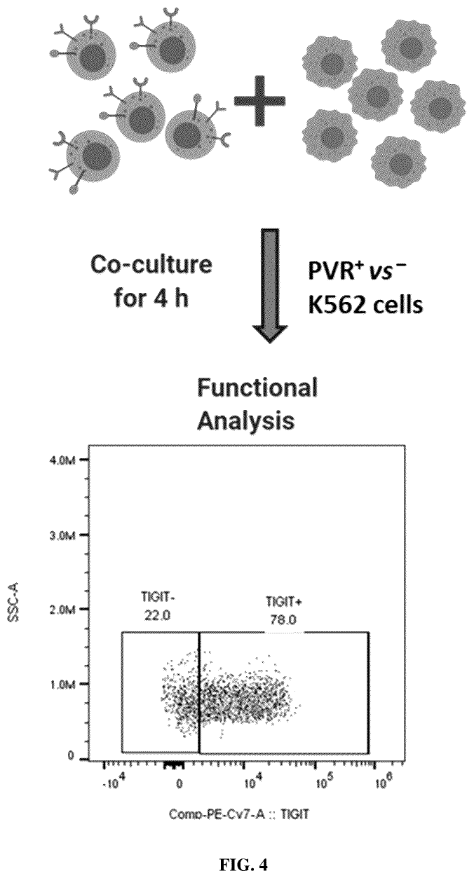

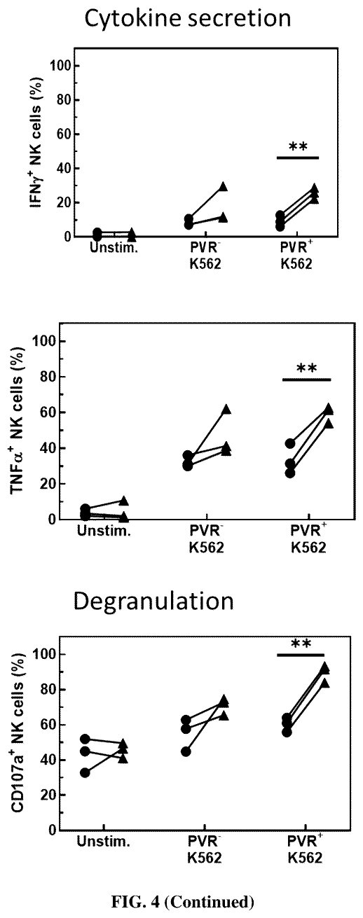

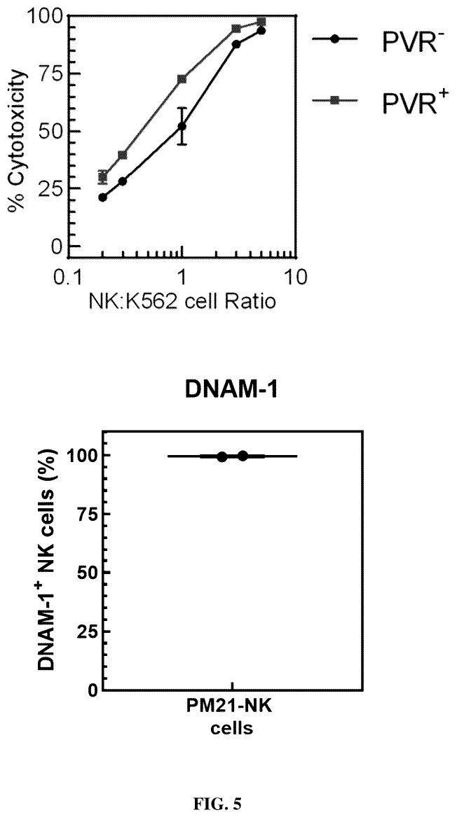

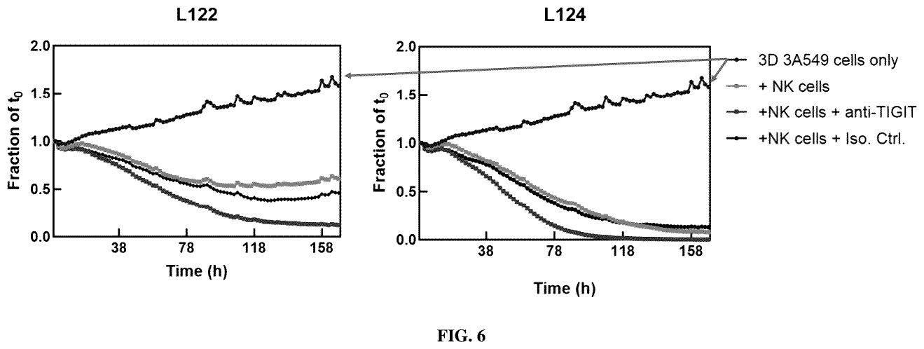

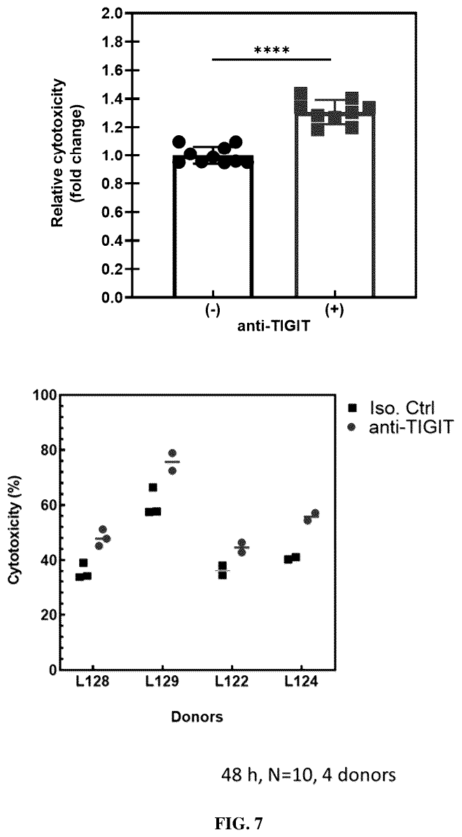

IFNγ and TNFα expression, and degranulation. 30,000 NK cells were co-cultured with PVR⁻ or PVR⁺ K562 cells in the presence of Ultra-LEAF isotype or anti-TIGIT antibodies (Biolegend, San Diego, CA, USA) for 4-6 hours in the presence of Brefeldin A and Golgi Stop™ at 37° C. Samples were stained with extracellular target protein-specific antibodies (CD3, CD56 and CD107a). NK cells were then fixed and permeabilized (eBiosciences IC Fixation and permeabilization buffers) and stained for intracellular protein targets (IFNγ and TNFα). Data was acquired by flow cytometry and analyzed by FlowJo software. See FIG. 4.

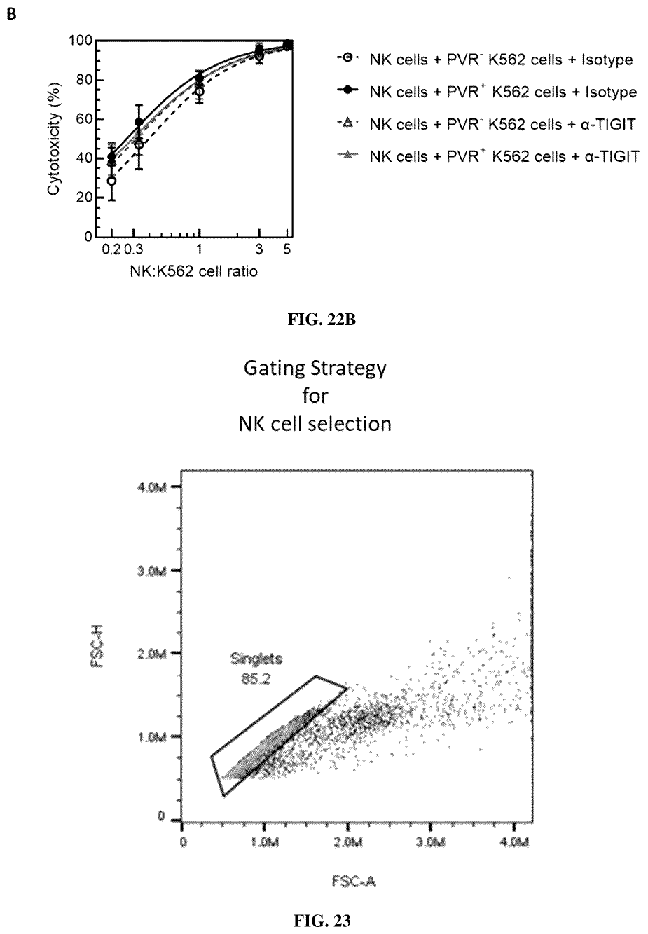

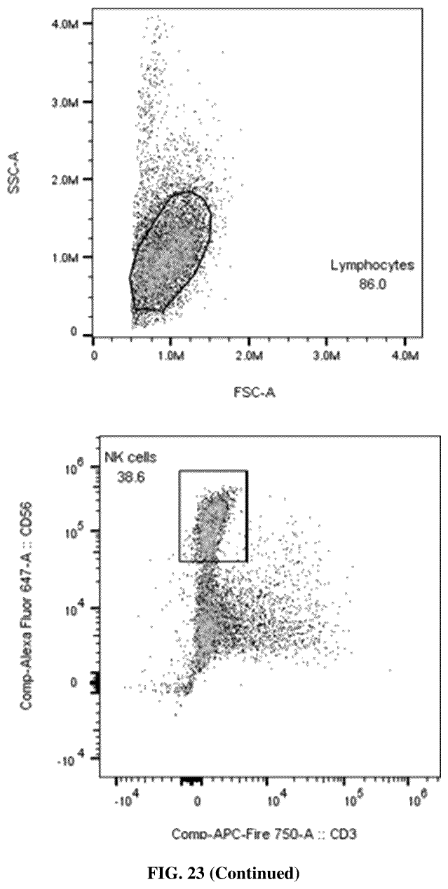

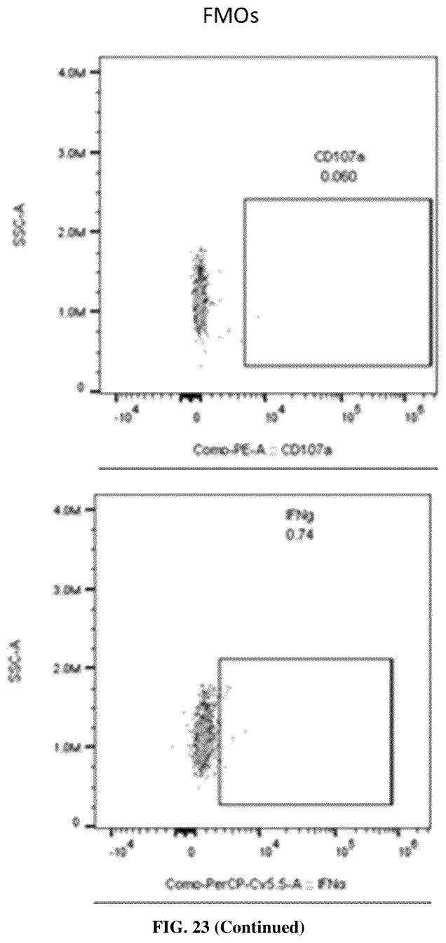

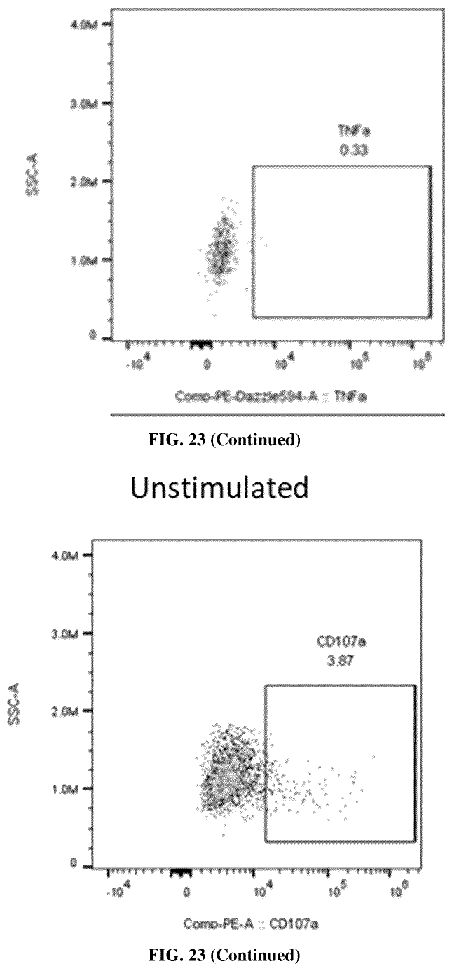

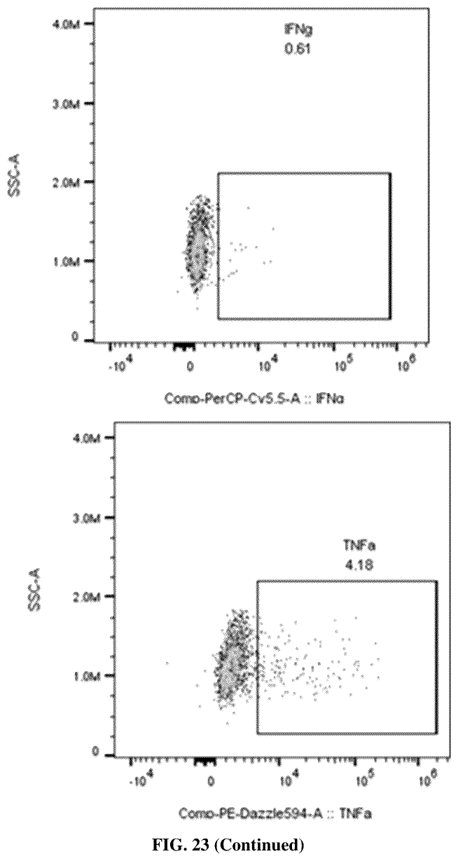

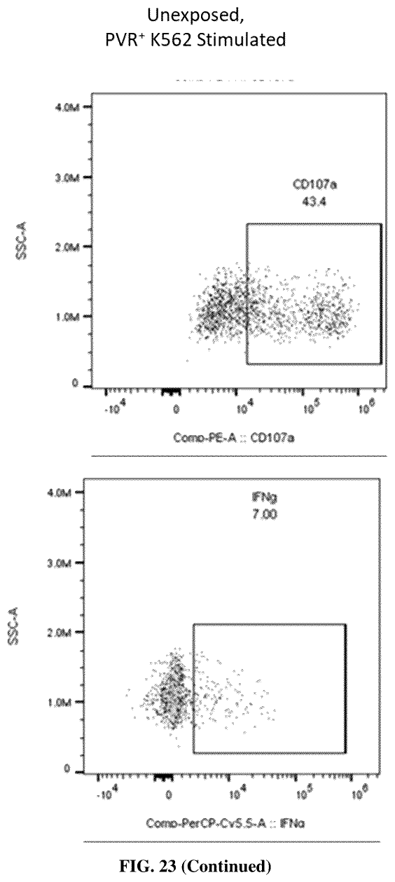

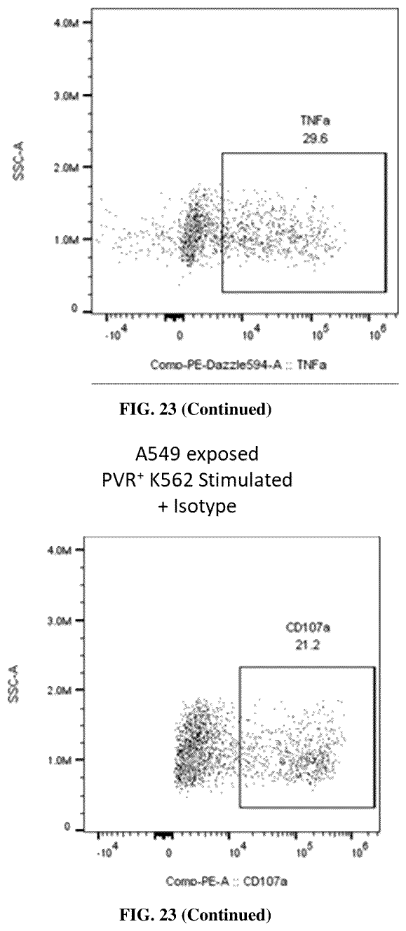

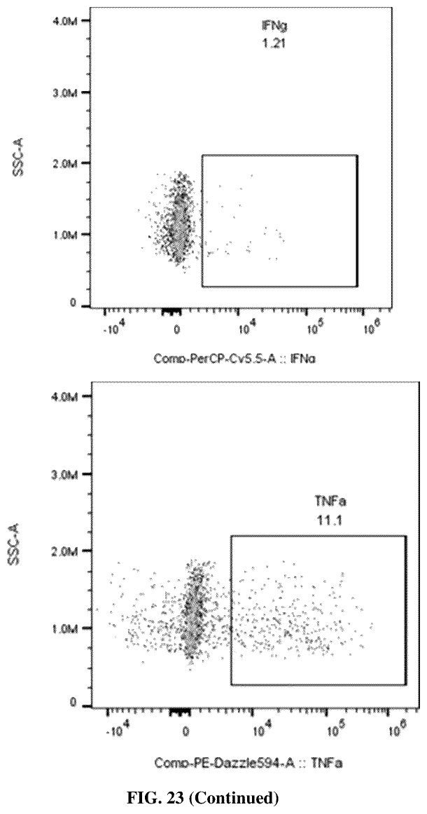

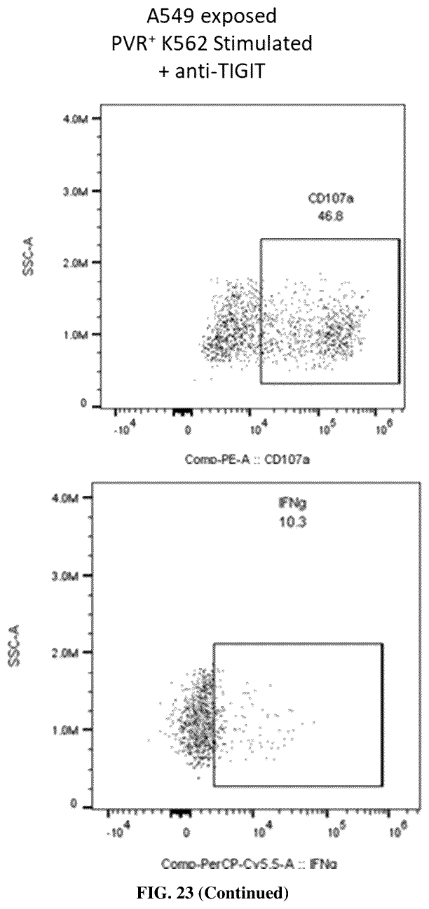

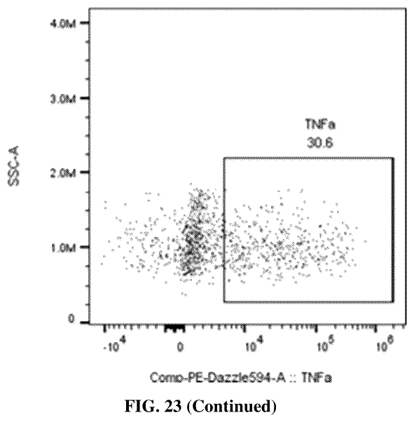

In vitro exhaustion model. A549-NLR cells (5000/well) were seeded in a 96-well clear round bottom ultra-low attachment microplate (Corning, Corning, NY, USA), centrifuged at 130×g for 10 minutes, and incubated for 3-4 days to form spheroids. NK cells were then added in the presence of Ultra-LEAF isotype or anti-TIGIT antibodies (Biolegend, San Diego, CA, USA). After 7 days of incubation, NK cells were stimulated with PVR⁻ or PVR+K562-GFPLuc cells for 4-6 hours in the presence of Brefeldin A (eBioscience, San Diego, CA, USA) and Golgi Stop™ (BD Biosciences, Franklin Lakes, NJ, USA). Samples were harvested and stained with CD56, CD3 and CD107a antibodies, fixed and permeabilized (eBioscience IC Fixation and Permeabilization buffers), and probed with antibodies for IFNγ and TNFα followed by analysis using flow cytometry. Representative gating strategies are shown in FIG. 23.

RNA-seq. NK cells were set up as described in exhaustion model. After 7 days of coincubation, NK cells were isolated with an NK cell selection kit (EasySep CD56+ selection kit; StemCell technologies) and analyzed by flow cytometry (Northern Lights 2000 Full Spectrum, Cytek) to determine NK cell purity. Total RNA was extracted from the NK cells (Direct-zol microprep kit, Zymo research). RNA quality (RIN value) was determined by TapeStation and used for polyA selection, library preparation, and RNA sequencing (Genewiz, Inc, South Plainfield, NJ). Raw RNA-seq data (Fastq) were analyzed with FastQC for quality control. Trimmomatic was used for trimming adaptor and low-quality reads. HISAT2 was used for mapping genes with hg38 human genome and Stringtie was used for assembly and quantification of read counts. Combat-seq was used to remove batch effects among samples. EdgeR was used to normalize gene expression and determine differentially expressed genes. Fold changes and P value of individual genes obtained from EdgeR were multiplied to make ranked gene list and was used for Pre-ranked Gene Set Enrichment (GSEA) analysis to determine enriched hallmark gene sets.

Statistical analysis. Statistical analysis was performed by GraphPad Prism 9.3.1. Paired or unpaired two-tailed Student's t test was used to analyze TIGIT expression, cytotoxicity and functional assays. All experiments were performed for at least 3 biological replicates. P value less than 0.05 was considered as statistically significant. P values are shown as * if p<0.05, ** if p<0.01, *** if p<0.001, **** if p<0.0001.

### Results

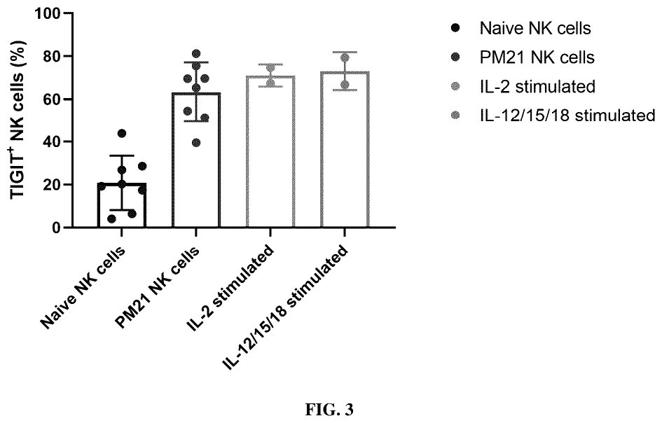

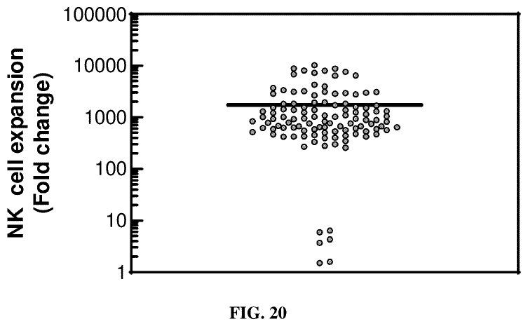

PM21-particle expanded or cytokine activated NK cells highly express TIGIT. NK cells obtained from healthy donors were expanded using PM21-particles (19,20). This feeder cell-free expansion technology utilizes plasma membrane particles derived from K562-mbIL21-41BBL cells (PM21-particles) to stimulate NK cell proliferation, resulting in an average 1,700-fold expansion of NK cells in 2 weeks (N=113 from 18 donors, FIG. 20). Resting NK cells isolated from PBMCs and NK cells expanded from matching donors using PM21-particles (PM21-NK cells) were analyzed for TIGIT expression by qRT-PCR and flow cytometry. TIGIT was upregulated on RNA level in PM21-NK cells as compared to resting NK cells (6±3-fold; p=0.04) (FIG. 2, left panel). Furthermore, the percentage of TIGIT⁺ NK cells was increased in PM21-NK cells as compared to resting NK cells (N=8, p<0.0001), with the expression ranging from 2% to 44% in resting NK cells and 39% to 88% in PM21-NK cells (FIG. 2, right panel). To determine if other NK cell activation methods also induce TIGIT expression, T cell-depleted PBMCs were stimulated overnight with either IL-2 (1000 U/mL) or the combination of IL-12 (10 μg/mL), IL-15 (100 μg/mL) and IL-18 (50 μg/mL) and the percentage of NK cells expressing TIGIT was compared to that of resting NK cells. TIGIT expression was upregulated on IL-2 activated NK cells (69±3%, p<0.0001) and IL-12/15/18 activated NK cells (60±15%, p=0.0001) compared to resting NK cells (21±13%) and was comparable to the level of expression on PM21-NK cells (67±16%) (FIG. 3). All together, these findings demonstrate that NK cells, upon expansion with PM21-particles or cytokine activation, upregulate TIGIT on both RNA and surface protein levels.

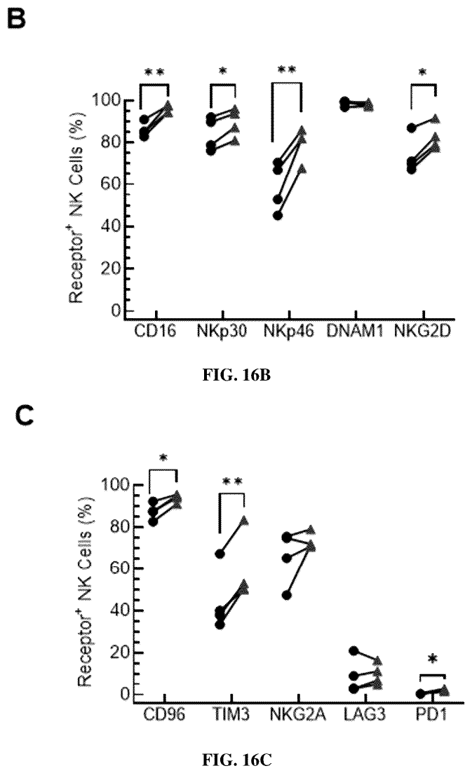

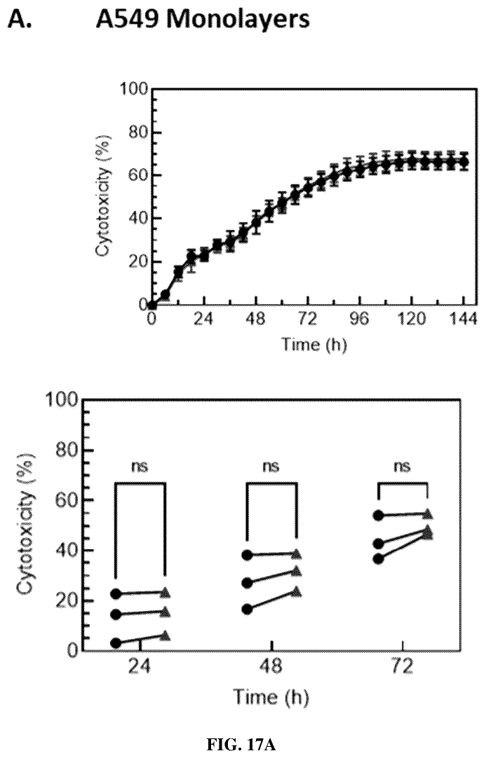

TIGIT positive PM21-NK cells express higher levels of activating and inhibitory receptors compared to TIGIT negative PM21-NK cells. To determine if the expression of TIGIT is associated with any changes to the phenotype and/or to the activation state of NK cells, the level of expression of major activating and inhibitory receptors was compared between TIGIT⁺ and TIGIT⁻ PM21-NK cells. Differences were evaluated on cells prior to day 14 of expansion when the expression is not at the maximal level and differential expression still can be assessed. In general, expression of activating and other inhibitory receptors was increased on TIGIT⁺ PM21-NK cells as compared to TIGIT⁻ PM21-NK cells, summarized in FIG. 16A. The percent of NK cells expressing the activating receptors CD16, NKp30, NKp46, DNAM-1, and NKG2D varied between donors, but was increased (p=0.02 or less) for TIGIT⁺ vs. TIGIT⁻ NK cells in donor-matched pairs for all activating receptors, except for DNAM-1. DNAM-1 was ubiquitously and highly expressed on all PM21-NK cells, averaging more than 98% of both TIGIT⁻ and TIGIT⁺ NK cells (FIG. 16B). The inhibitory receptors CD96, TIM-3, NKG2A, and LAG-3 were also expressed on PM21-NK cells, while PD-1 was detected on fewer than 2% of PM21-NK cells (FIG. 16C). A significantly higher percentage of TIGIT⁺ NK cells expressed CD96 (p=0.01), and TIM-3 (p=0.003) compared to donor-matched TIGIT⁻ NK cells while expression of NKG2A and LAG-3 were not significantly different between TIGIT⁺ and TIGIT⁻ NK cells. These findings indicates that TIGIT⁺ NK cells represent a more activated cell subpopulation, expressing higher levels of important activating and some inhibitory receptors, typically induced upon activation.

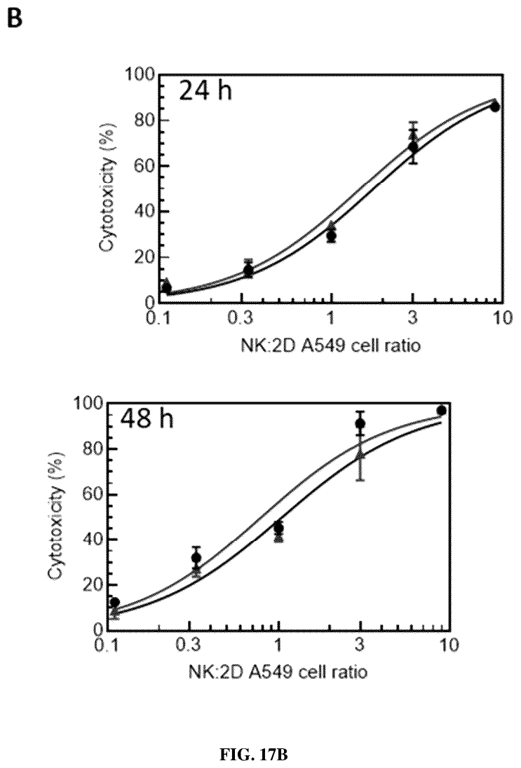

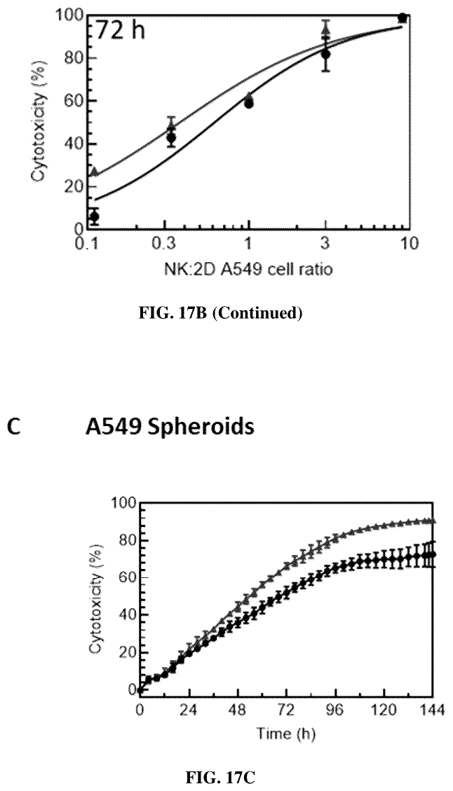

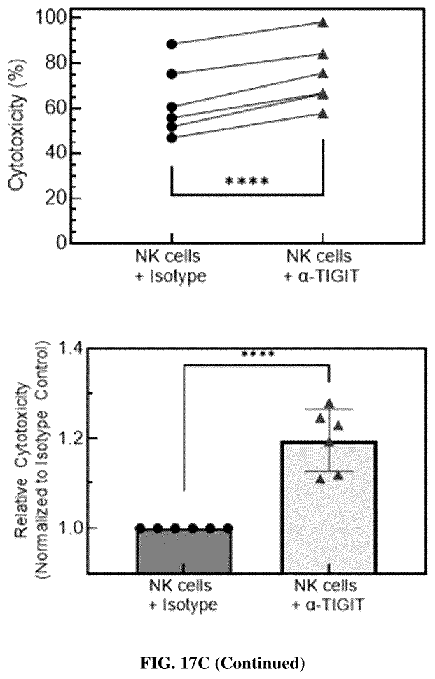

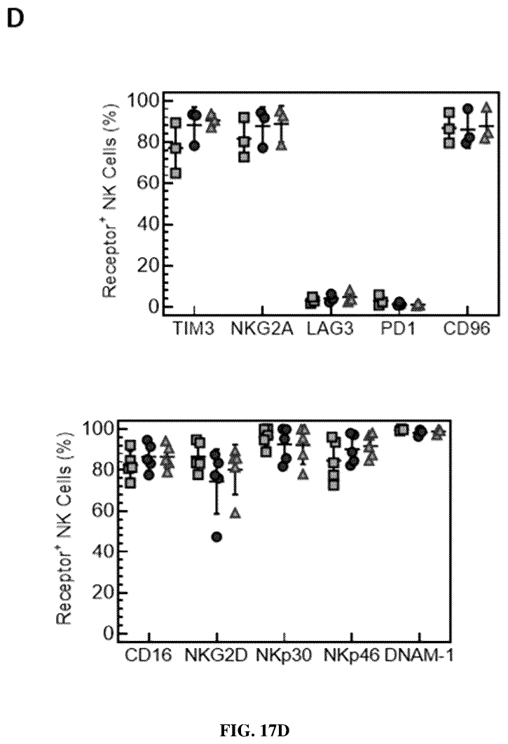

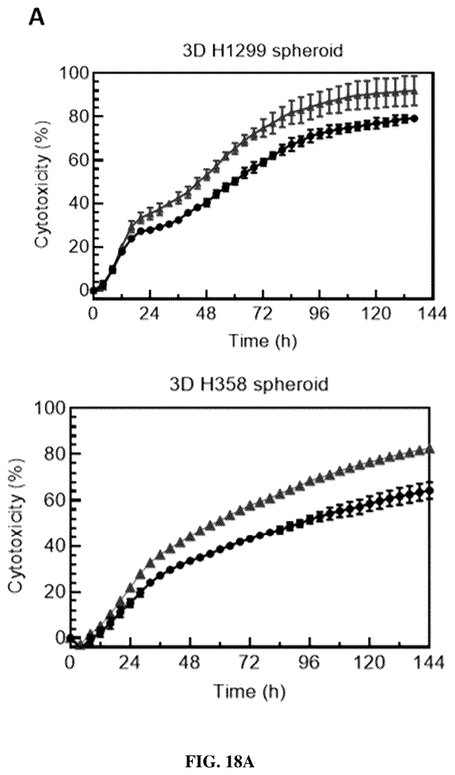

TIGIT blockade enhances PM21-NK cell cytotoxicity against 3D lung tumor spheroids. To access the effect of TIGIT blockade on PM21-NK cell anti-tumor functions, PM21-NK cell cytotoxicity against A549 lung tumor cells was examined. This cell line expresses PVR and PVRL2 but not the PVRL4 that can bind TIGIT (Table 1). PM21-NK cells from 3 donors were co-cultured with 2D A549 lung cancer cell monolayers at a 0.33:1 NK:A549 ratio in the presence of anti-TIGIT antibodies or isotype controls and cytotoxicity was measured with a live-cell imaging assay. No significant enhancement of PM21-NK cell killing occurred with TIGIT blockade over 72 h (cytotoxicity in the presence of isotype control vs. anti-TIGIT antibodies was 14±10% vs. 15±9% at 24 h, 27±11% vs. 31±7% at 48 h, and 44±9% vs. 50±4% for 72 h) (FIG. 17A). Concentration-dependent cytotoxicity curves were also determined for each of these timepoints for one donor and no difference in killing was observed with or without TIGIT blockade (559±18% ratio vs. 525±24% ratio at 24 h, 689±17% ratio vs. 629±37% ratio at 48 h, and 751±14% ratio vs. 697±25% ratio at 72 h) (FIG. 17B).

TABLE 1
TIGIT ligands are expressed in several lung cancer cell lines. Lung cancer cell
lines A549, NCI-H358, NCI-H1299, and NCI-H1975 were stained with TIGIT ligand
specific antibodies and compared to isotype controls to determine ligand expression
by flow cytometry. Data are presented as percent of cancer cells that are ligand
positive (%) and Mean Fluorescent Intensity (MFI) averaged from two different passages.
TIGIT Ligand Expression in Lung Cancer Cell Lines

| | PVR | PVR | PVRL2 | PVRL2 | PVRL4 | PVRL4 |
|---|---|---|---|---|---|---|
| | (%) | (MFI, ×10²) | (%) | (MFI, ×10²) | (%) | (MFI, ×10²) |
| A549 | 100 | 2356 ± 11 | 100 | 324 ± 13 | 1 | 27 ± 6 |
| NCI-H358 | 100 | 2319 ± 6 | 100 | 173 ± 0.2 | 97 | 598 ± 1 |
| NCI-H1299 | 100 | 3863 ± 2 | 100 | 770 ± 15 | 2 ± 1 | 43 ± 6 |
| NCI-H1975 | 100 | 4150 ± 71 | 100 | 129 ± 11 | 28 | 108 ± 5 |

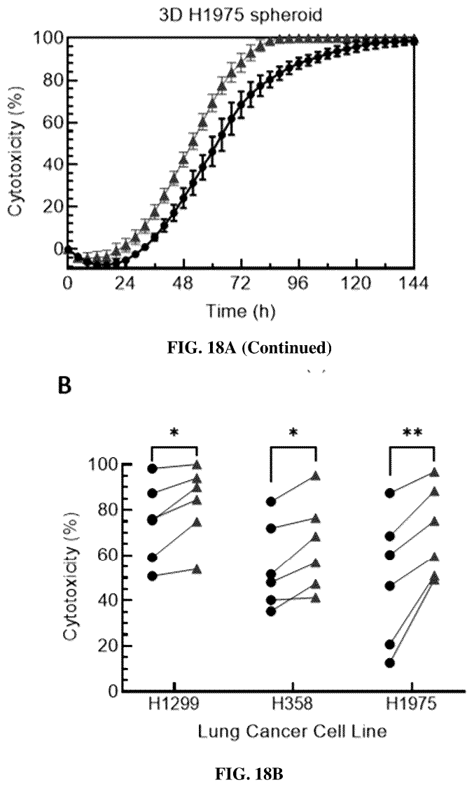

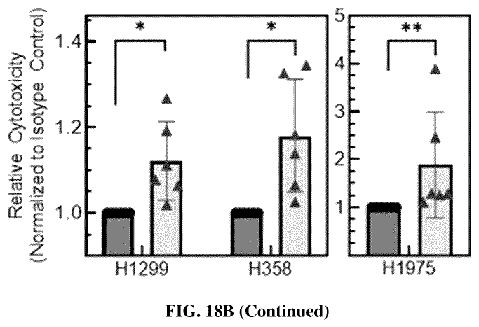

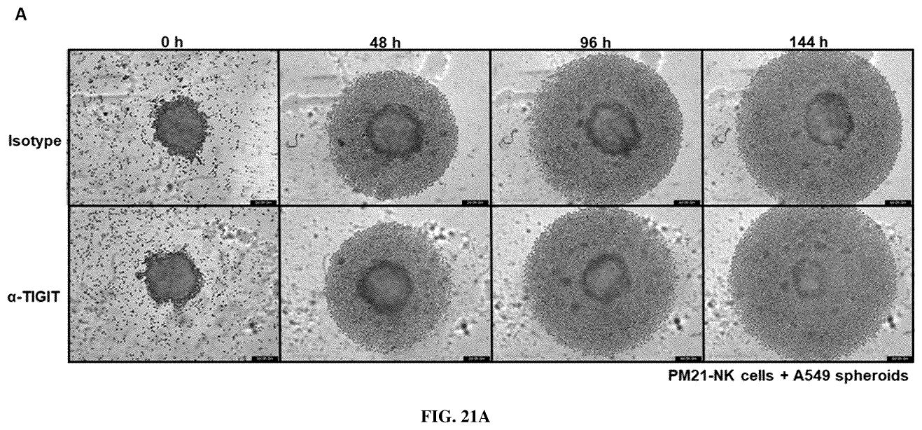

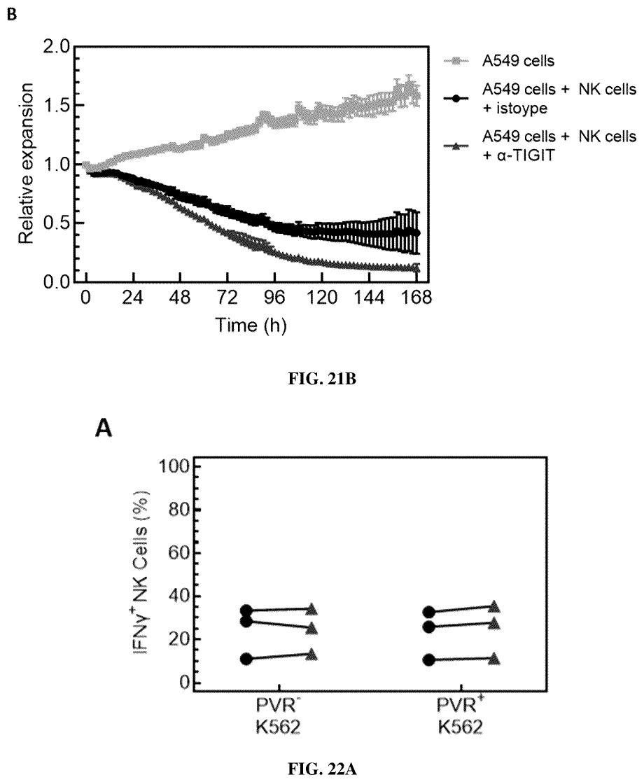

To determine if blockade of TIGIT affects PM21-NK cell cytotoxicity during exposure to lung cancer spheroids that better mimic the cancer environment, PM21-NK cells were cocultured with A549 cell spheroids and their killing over time, as well as NK cell phenotype at the end of the co-culture, were assessed. PM21-NK cells were exposed to the tumor spheroid for 7 days in the presence of anti-TIGIT or isotype control antibodies. Representative images at several time points show increased killing of A549 spheroids in the presence of anti-TIGIT, apparent by the smaller size of resulting spheroids (FIG. 21A) and reduced relative expansion (FIG. 21B). Cytotoxicity curves over time were determined and representative curves from one donor and summary cytotoxicity data from multiple donors are shown in FIG. 17C. TIGIT blockade enhanced PM21-NK cell cytotoxicity against A549 spheroids at 72 h by an average of 20% across donors (p<0.0001, 1:1 PM21-NK cells:A549 cells, N=6) (FIG. 17C). Expression of multiple activating and inhibitory receptors was evaluated on unexposed control PM21-NK cells and PM21-NK cells from 3 donors after exposure to A549 spheroid in presence or absence of anti-TIGIT antibody. Tumor exposure alone or with TIGIT blockade did not change the frequency of NK cells expressing inhibitory receptors TIM-3, NKG2A, LAG-3, PD-1 or CD96 compared to isotype control or unexposed NK cells (FIG. 17D). Similarly, there was no difference in expression of activating receptor CD16, NKG2D, NKp30, NKp46 and DNAM-1 after tumor exposure with or without TIGIT blockade, although there was a trend toward decreased expression of NKG2D upon spheroid exposure particularly with isotype control (FIG. 17D). To confirm that the enhancement in NK cell cytotoxicity against lung cancer spheroids upon TIGIT blockade is not isolated to a single cell line, testing of the anti-TIGIT antibody was also performed in NCI-H1299, NCI-H358, and NCI-1075 lung cancer cell lines. These cell lines also highly express PVR and PVRL2 while H358 also expresses PVRL4 among the TIGIT ligands tested (Table 1). PM21-NK cell cytotoxicity against spheroids of these cell lines was determined using a live-cell imaging assay. Each cell line had different rates at which the target cells were killed, but all demonstrated enhanced cytotoxicity upon TIGIT blockade (FIG. 18A). While there was donor-dependent variability in the extent of killing, TIGIT blockade increased cytotoxicity in donor-matched comparisons against all cell lines tested (NCI-H1299 p=0.02, NCI-H358 p=0.01, NCI-1975 p=0.005) and enhanced killing of H1299 by 12% on average, H358 by 18%, and H1975 by a striking 88% (FIG. 18B). Collectively, these findings indicate that in 3D spheroid models, TIGIT blockade enhances cytotoxicity of PM21-NK cells against lung cancer cells without a change in the phenotype of the NK cells.

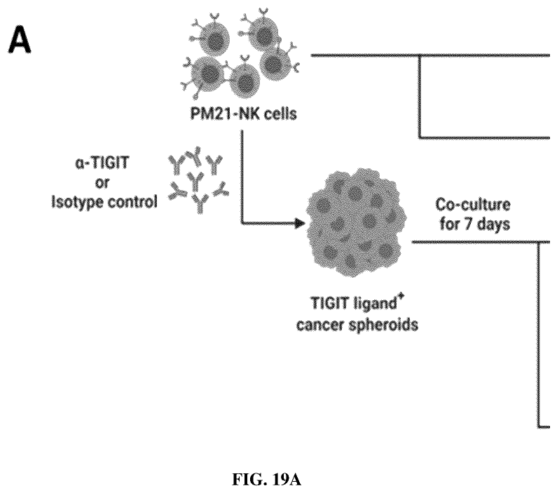

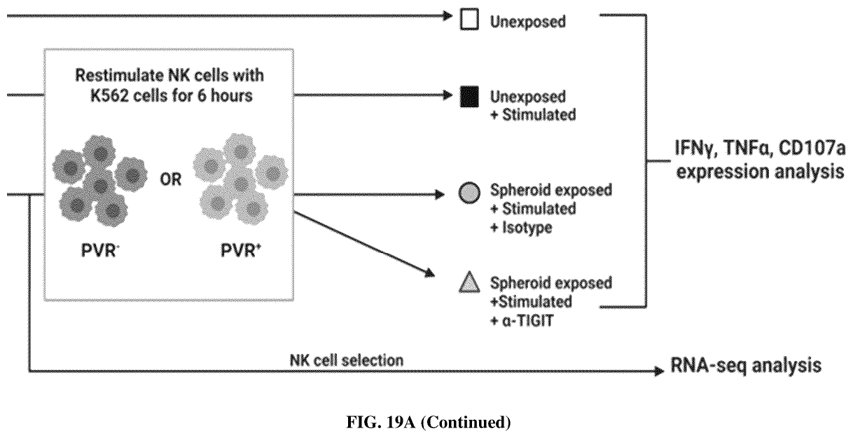

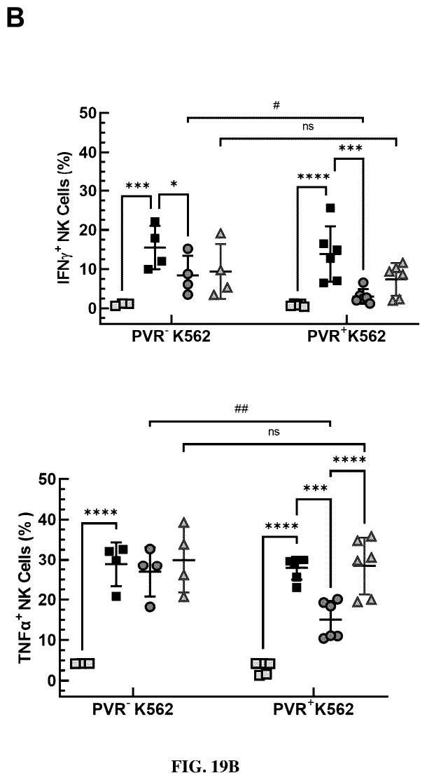

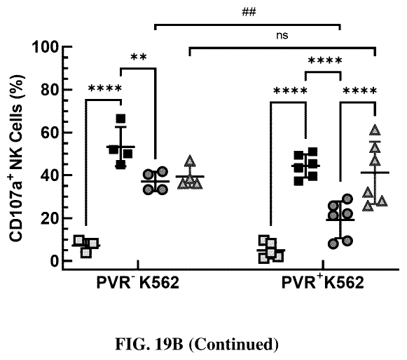

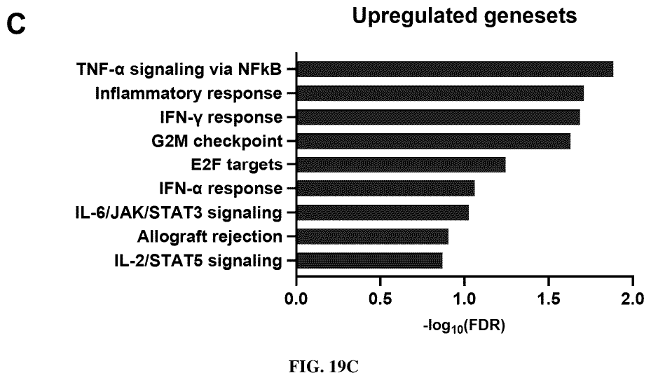

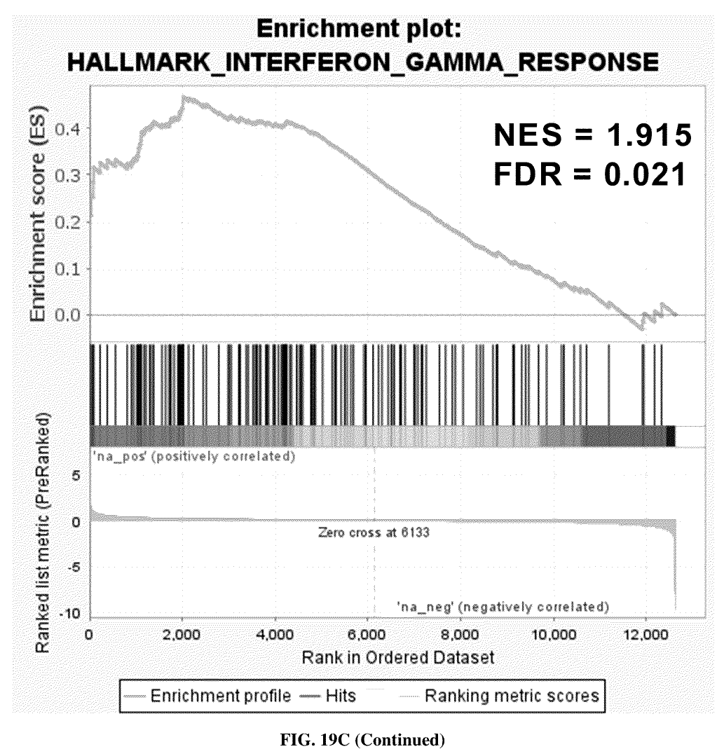

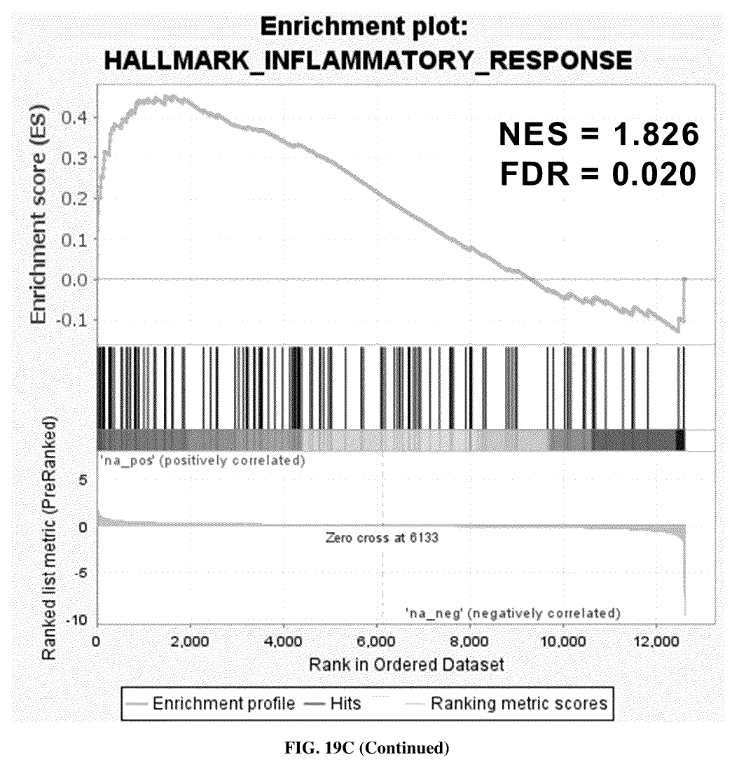

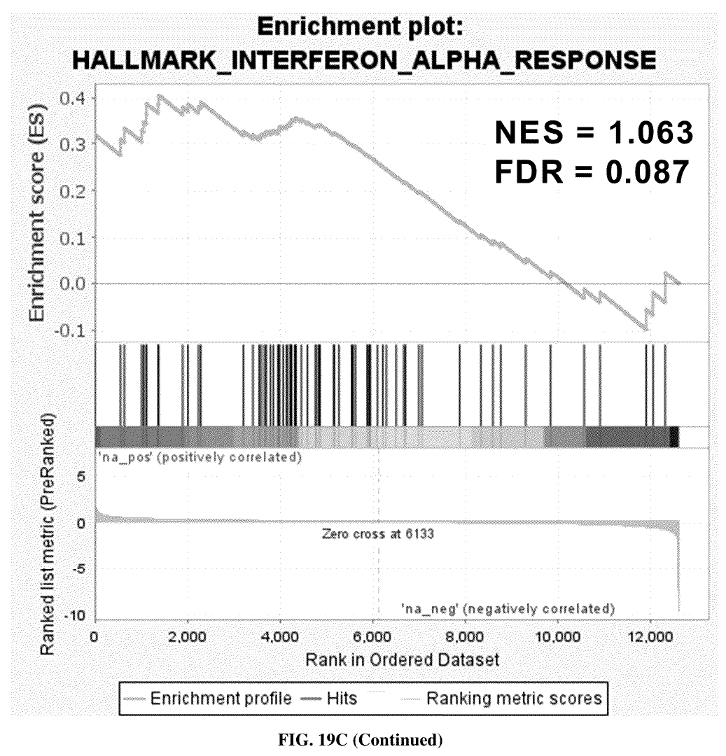

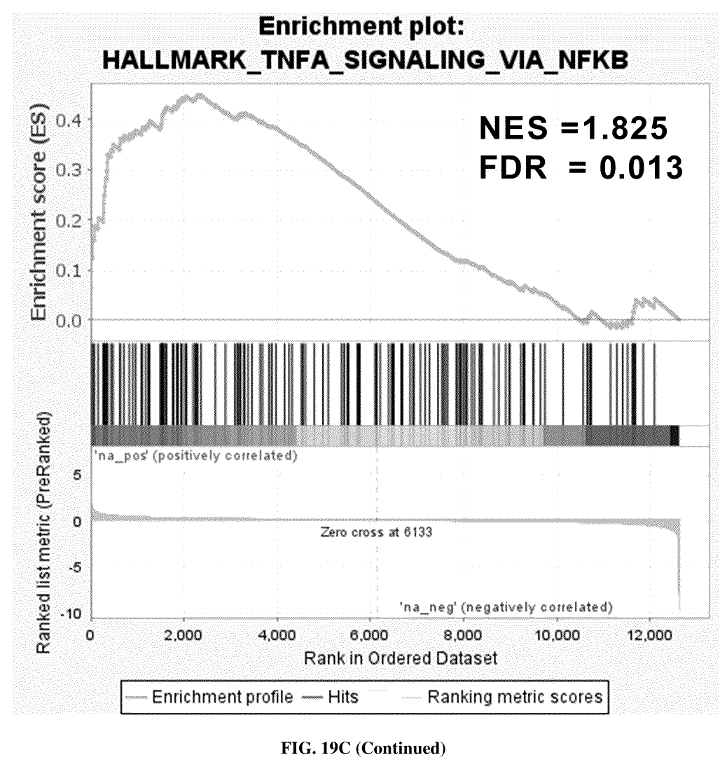

TIGIT blockade preserves PM21-NK cell effector function against PVR positive cancer cells after co-culture with cancer cell spheroids. NK cell exhaustion can occur in the context of the tumor microenvironment whereby long-term exposure to tumors can lead to decreased effector function, altered phenotype, or decreased killing. The mechanisms leading to NK cell exhaustion are not well defined, however recent studies have shown a role for exacerbated inhibitory receptor signaling. Previous studies have reported that chronic inhibitory signaling promotes exhaustion of cytotoxic immune cells and TIGIT has been associated with NK cell exhaustion in tumor-bearing mouse models and cancer patients. To determine if TIGIT blockade can alleviate signs of exhaustion in PM21-NK cells upon exposure to tumor spheroids, an in vitro exhaustion model was developed. PM21-NK cells were first co-cultured with A549 spheroids for 7 days either in the presence of anti-TIGIT or isotype control antibodies. NK cells were then stimulated with either PVR⁻ or PVR⁺ K562 cells and production of effector cytokines and degranulation were assessed. Unexposed, unstimulated PM21-NK cells were used as a negative control while unexposed PM21-NK cells, stimulated with either PVR⁺ K562 or PVR⁻ K562 were used as positive controls. A schematic of the in vitro exhaustion model is shown in FIG. 19A.

When unexposed PM21-NK cells were stimulated with K562 cells, the percentage of NK cells expressing IFNγ increased from less than 1% to 15±5% (p=0.0002) when stimulated with PVR⁻ K562 cells and 14±7% (p<0.0001) with PVR⁺ K562 cells (FIG. 19B). Restimulation with K562 cell after A549 spheroid co-culture, resulted in lower percentage of PM21-NK cells expressing IFNγ (8±5% for PVR⁻; p=0.03 and 4±3% with PVR⁺ cells; p=0.0002) (FIG. 19B) and the decrease was greater when PVR⁺ cells were used (p=0.04). TIGIT blockade during co-culture with A549 spheroids did not mitigate the tumor induced decrease in IFNγ expression after restimulation with PVR⁻ cells with still only 9±7% of cells expressing IFNγ. In contrast, restimulation with PVR⁺ K562 cells, increased IFNγ expression in NK cells from 3±2% with isotype control to 7±4% with anti-TIGIT, although did not reach significance (p=0.1) (FIG. 19B). Additionally, there was no longer a significant difference in IFNγ expression between PVR⁻ or PVR⁺ K562 cell restimulation when anti-TIGIT antibodies were present in the initial tumor co-culture, indicating the initial difference was TIGIT dependent. Thus, tumor exposure resulted in close to 80% loss in the IFNγ generation capacity with about 30% of this loss being dependent on TIGIT/PVR engagement.

TNFα generation was also negatively impacted by tumor exposure, with most of the decrease being driven by the TIGIT/PVR axis. Stimulation of unexposed PM21-NK cells with K562 cells resulted in an increase in the frequency of TNFα+NK cells as compared to unstimulated cells (4±1% vs. 29±5% upon PVR⁻ K562 cell stimulation; p<0.0001 and 4% vs. 28±3% with PVR⁺ K562 cells; p<0.0001) (FIG. 19B). Compared to unexposed cells, A549 tumor exposure either in the presence of isotype control or anti-TIGIT antibodies did not result in change of the frequency of TNFα+NK cells induced in response to PVR⁻ K562 cell restimulation (27±6% in the presence of isotype control and 30±8% with anti-TIGIT antibodies) (FIG. 19B). However, restimulation with PVR⁺ K562 cells resulted in a lower frequency of TNFα+NK cells after co-cultures with isotype control antibodies as compared to PVR⁻ K562 cell restimulation (27±6% with PVR⁻ cells vs. 15±5% with PVR⁺ cells; p=0.008), or as compared to unexposed NK cells stimulated with PVR⁺ K562 cells (28±3% in unexposed vs. 15±5% for spheroid exposed; p=0.0001) (FIG. 19B). Blocking of TIGIT during spheroid co-culture prevented the decrease in the frequency of TNFα+NK cells, with 28±7% of NK cells expressing TNFα after restimulation with PVR⁺ K562 cells; frequencies that were comparable to unexposed stimulated NK cells (28±3%) and to exposed NK cells stimulated with PVR⁻ K562 cell (27±6%) (FIG. 19B). Thus, blocking TIGIT fully restored the TNFα production capacity of NK cells.

TIGIT blockade also restored most of the ability of PM21-NK cells to degranulate upon PVR⁺ cell restimulation post-tumor co-culture. Surface CD107a expression, a marker for degranulation, increased in unexposed PM21-NK cells upon stimulation with K562 cells where the frequencies of degranulating, CD107a⁺ NK cells increased from 6±3% in unstimulated to 53±9% (p<0.0001) when stimulated with PVR⁻ K562 cells and to 44±5% (p<0.0001) after PVR+K562 cell stimulation (FIG. 19B). Tumor co-culture in the presence of isotype control antibodies decreased the frequency of CD107a⁺ NK cells upon restimulation with K562 cells (37±5% for PVR⁻; p=0.004 and 19±8% PVR⁺; p<0.0001) with a larger decrease with PVR+K562 cell compared PVR⁻ cells (p=0.005). TIGIT blockade during tumor co-culture only restored CD107a expression for PVR⁺ K562 restimulated NK cells; resulting in an increase in the frequency of CD107a⁺ NK cells as compared to isotype control conditions (41% with TIGIT blockade vs. 19% with isotype control; p<0.0001), resulting in comparable frequencies of CD107a⁺ NK cells to those observed for PVR⁻ K562 restimulated NK cells, which remained unchanged after TIGIT blockade (41% with PVR⁺ cell restimulation vs. 39% PVR⁻ cell restimulation).

In summary, evidence of NK cell exhaustion upon tumor was observed resulting in decreased frequencies of NK cells producing IFNγ, TNFα, and degranulating, after re-stimulation with K562 cells compared to stimulation of unexposed NK cells with K562 cells and these decreases were greater when restimulated with PVR⁺ K562 cells. TIGIT blockade restored the ability of NK cells to produce IFNγ, TNFα, and degranulate CD107a upon restimulation with PVR⁺ cell in tumor-exposed PM21-NK cells back to levels comparable to re-stimulation with PVR⁻ K562 cells or to those observed for unexposed NK cells stimulated with PVR⁺ K562 cells.

In order to determine if the protective effect of TIGIT blockade on the effector functions of PM21-NK cells post-exposure occurs on a transcriptional level, NK cells were selected after co-culture with A549 spheroids for 7 days and RNA extracted for sequencing and transcriptomic analysis (schematic depicted in FIG. 19A). Gene set enrichment analysis revealed that TIGIT blockade upregulated hallmark gene sets including TNFα signaling via NFkB, inflammatory response, IFNγ response and IFNα response gene sets (FIG. 19C). These enrichments in the transcriptome indicate a more activated state (57) of PM21-NK cells upon TIGIT blockade after A549 co-culture. All together, these observations from functional and transcriptomic analysis demonstrate that TIGIT blockade restores PM21-NK cell anti-tumor functions against PVR positive cancer cells after long-term exposure to cancer cell spheroids.

Ex vivo expanded-NK cells have emerged as a promising cancer immunotherapy platform due to their broad anti-tumor function. Previous clinical studies demonstrated that ex vivo expanded-NK cells and CAR-NK cells are safe as cancer therapy with no induction of GVHD. However, NK cells express multiple inhibitory receptors, such as TIGIT, which can be exploited by cancer cells in the tumor microenvironment to suppress NK cell anti-tumor activities and may limit the efficacy of NK cell-based cancer immunotherapy. Thus, understanding inhibitory receptor signaling and developing therapeutic blockade strategies may further improve anti-tumor responses.

Previous studies showed that TIGIT signaling regulates multiple steps of the cancer immunity cycle and TIGIT blockade significantly reduces tumor growth and survival in preclinical studies. TIGIT⁺ NK cells exhibit more exhaustive phenotypes in several cancers. and blockade of TIGIT improves NK cell anti-tumor activity against cancer cells. Studies on the effects of TIGIT blockade have been thus far limited to preclinical mouse models and mechanistic in vitro studies characterizing TIGIT blockade in human NK cells are limited. In this study, the effect of blockade of TIGIT signaling in ex vivo expanded PM21-NK cells was characterized and tested in a novel in vitro model for NK cell exhaustion.

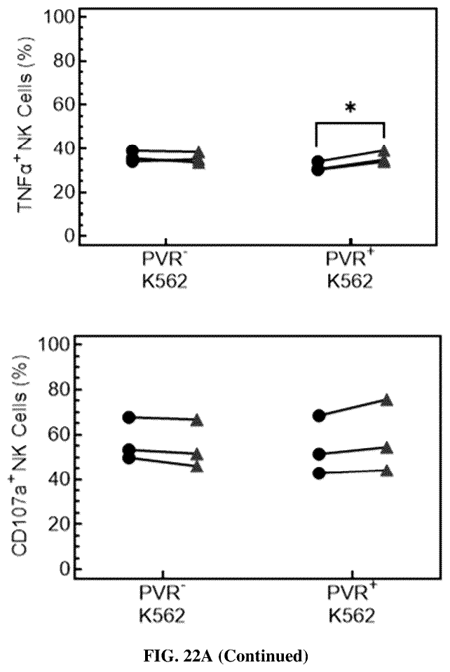

TIGIT was found to be highly expressed on the surface of PM21-NK cells. Not only PM21-NK cells, but also NK cell activated with IL-2 or IL-12/15/18 upregulate TIGIT expression. This indicates that TIGIT is a marker of NK cell activation. Previous studies reported similar findings where IL-15 stimulation increased TIGIT expression on NK cells. Phenotypic analysis revealed that TIGIT⁺ PM21-NK cells express higher level of other inhibitory and activating receptors further supporting that TIGIT expression is a consequence of NK cell activation through expansion. Surprisingly, TIGIT blockade had no effect on PM21-NK cell cytotoxicity in short-term assays. This indicates that DNAM-1-PVR driven activation overpowers TIGIT inhibitory signaling in PM21-NK cells in the short-term. To better mimic chronic stimulation of TIGIT in tumor environment and assess its blockade, cytotoxicity assays were carried out using tumor spheroids. TIGIT blockade improved PM21-NK cell cytotoxicity against multiple lung cancer spheroids. This indicates that during long-term exposure to tumor spheroid, TIGIT signaling can inhibit NK cell-mediated killing. To further characterize the effect of TIGIT signaling on the effector functions of NK cells after long-term exposure to tumor, an in vitro exhaustion model was developed. After exposure to A549 tumor spheroids for 7 days with anti-TIGIT or isotype control antibodies, PM21-NK cells were re-challenged with either PVR⁻ or PVR⁺ K562 cells to discern the contribution of PVR/TIGIT axis to the decline of each of the effector functions. From this model, signs of NK cell exhaustion were evident by decreased expression of effector cytokines IFNγ and TNFα, and reduced degranulation, as measured by surface CD107a expression, upon re-exposure and were to a different degree a consequence of chronic TIGIT engagement indicating that TIGIT is a major but not the only contributor to NK cell exhaustion. Decrease in TNFα expression upon restimulation was only observed when tumor-exposed NK cells were re-challenged with PVR⁺ cells and the effect was fully mitigated with TIGIT blockade. This indicates the decrease in TNFα expression observed in this model is principally TIGIT driven. In contrast, IFNγ and CD107a expression in tumor-exposed NK cells showed decreased expression in response to both PVR⁻ and PVR⁺ restimulation, indicating additional mechanisms during tumor exposure led to the deficit in IFNγ production and degranulation. TIGIT blockade, however, fully restored expression levels to those comparable to the response to PVR⁻ cells. Of note, TIGIT blockade in unexposed NK cells, had no effect on IFNγ, TNFα, or CD107a expression upon stimulation with either PVR⁻ or PVR⁺ K562 cells (FIG. 22A). No effect on the cytotoxicity against either PVR⁻ or PVR⁺ K562 cells was observed either (FIG. 22B). The fact restorative effects were only seen in the long-term exposure model when the NK cells were restimulated with PVR⁺ cancer cells indicates that sensitization to TIGIT engagement occurs during tumor spheroid exposure.

Although decrease in effector function upon long term exposure was observed PM21-NK cells still retained some cytotoxic function with 50% fewer cells degranulating and producing TNFα upon restimulation with PVR⁺ K562 cells as compared to unexposed PM21-NK cells. In summary, this demonstrated that TIGIT blockade is an effective approach to alleviate TIGIT⁻driven exhaustion in NK cells exposed to tumor and can preserve effector functions against PVR⁺ cancer cells. Transcriptomics analysis of NK cells after exposure to the A549 spheroids confirmed TIGIT blockade upregulated gene sets involved in inflammatory responses and TNFα signaling and indicated a more activated state of these NK cells.

Previous studies showed that cancer cells acquire resistance to PD-1 signaling blockade and may upregulate TIGIT on T cells. This finding indicates that it is important to examine phenotypic changes after the TIGIT blockade of PM21-NK cells to find alternative receptors involved in further resistance. Interestingly, there were no significant changes of activating and inhibitory receptors on PM21-NK cells upon TIGIT blockade in long-term spheroid models. These observations indicate that, unlike PD-1 signaling, blockade of TIGIT signaling may be less prone to acquire alternative resistance mechanisms. Studies in multiple mouse models indicated that TIGIT blockade can prevent both NK and T cell exhaustion. The efficacy of anti-TIGIT antibody was dependent on NK cells and NK cells enhanced T cell anti-tumor activity. Thus, adoptive NK cells may further improve the efficacy of anti-TIGIT antibodies by boosting overall T cell immune response against cancer cells in cancer patients frequently lacking NK cell compartment.

In summary, this study provides insight into the molecular mechanisms of TIGIT blockade in ex vivo expanded-human NK cells. Anti-TIGIT antibodies can significantly improve the efficacy of PM21-NK cell tumor immunity and adoptive PM21-NK cells and anti-TIGIT antibodies can be used as a combination therapy against cancer, such as, lung tumors.

### Example 7. TIGIT KO NK Cells have Enhanced Cytotoxicity Against Lung Cancer Cells and are Resistant to Fratricide in Combination with Therapeutic TIGIT Antibodies

TIGIT blockade is promising new approach to cancer treatment and is currently undergoing late-stage clinical development in combination with PD-1/PD-L1 blockade. Preclinical studies indicated involvement of NK cells in anti-tumor response of TIGIT antibodies. Furthermore, murine studies evidenced that inclusion of ADCC-competent Fc in therapeutic antibodies targeting TIGIT was critical for anti-tumor response likely through depletion of TIGIT+ Tregs and/or myeloid cell activation via Fcγ engagement. Thus, most of therapeutic anti-TIGIT antibodies currently in development have Fc that are competent for and some further optimized to engage ADCC. Early clinical studies with Tiragolumab (Fc competent, Roche) showed that TIGIT significantly increases response rates and duration of response as compared to anti-PD-L1 alone (Atezolizumab) but only in patients with tumors having high frequency of PD-L1 positive cells (>50%). Although early results were highly encouraging, Tiragolumab failed in phase 3 studies in NSCLC and SCLC.

The majority of activated NK cells express high levels of TIGIT upon activation. Activated NK cells respond to tumors, secrete IFNγ which leads to induction of PD-L1 on targeted tumors. Thus, presence of activated NK cells through TIGIT blockade and induction of PD-L1 should lead to enhanced response to PD-(L)1/TIGIT blockade combo. Yet, binding of ADCC competent antibodies to TIGIT, which is highly expressed on activated NK cells can also lead to fratricide and NK cell depletion. Thus knock-out of TIGIT in NK cells prior to adoptive transfer can increase NK cell killing of tumors as well as prevent NK cell fratricide if used in combination with Fc competent anti-TIGIT therapeutics.

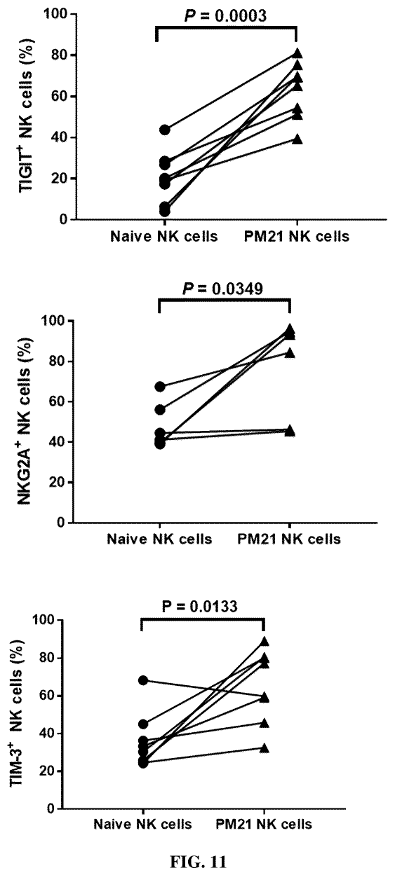

Evaluate the effect of Fc-competent TIGIT antibodies on NK cell fratricide. Previous studies examined that the efficacy of anti-TIGIT antibodies is through Fc-dependent depletion of TIGIT expressing Treg cells. Since PM21 NK cells highly express TIGIT, whether Fc-competent anti-TIGIT antibodies induce PM21 NK cell fratricide was tested. In this experiment, PM21 NK cells and TIGIT KO NK cells were incubated with Fc-competent anti-TIGIT antibodies (experimental), an anti-TIGIT antibody that is not Fc-competent, and with Fc-competent anti-CD38 (positive control) and absolute live NK cells were determined to understand the fratricide effect. PM21 NK cells alone can be used as negative control. FIGS. 9 and 10 show that Fc-competent (humanized) TIGIT antibodies (Tiragolumab) can induce moderate fratricide of PM21 NK cells but not for TIGIT KO PM21 NK cells.

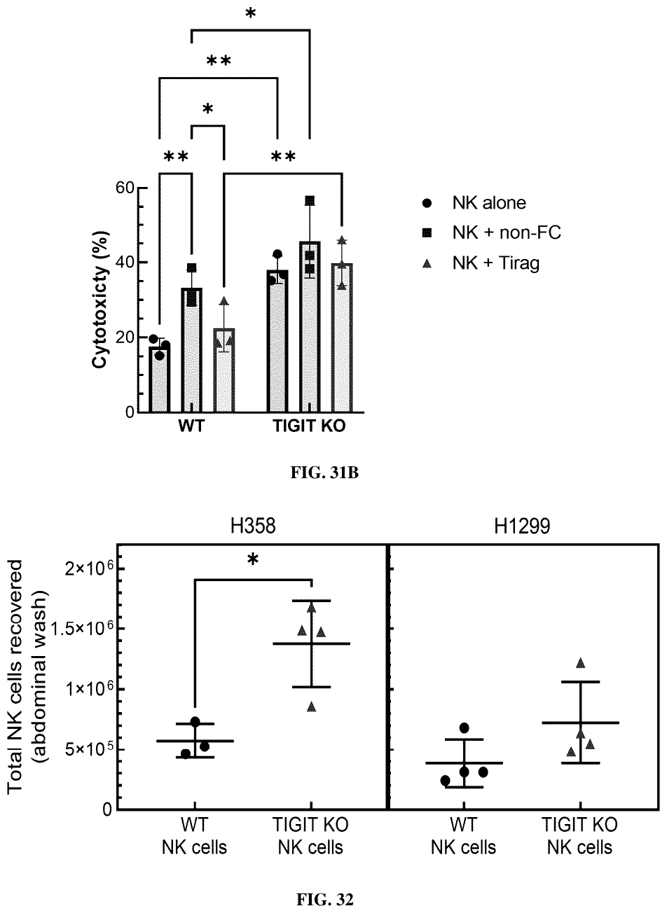

As shown in FIG. 31A, WT PM21-NK cells and TIGIT KO PM21-NK cells were co-cultured with non-Fc competent anti-TIGIT and Fc competent anti-TIGIT antibody (Tiragolumab) for 24 hours. NK cells were stained with Dioc6 and Draq7 to count viable NK cells. Tiragolumab induced significant fratricide against WT PM21-NK cells but not against TIGIT KO PM21-NK cells (N=3 donors, each in triplicate). Data are presented as scatter plots or bar graphs with error bars representing standard deviation. Statistical significance was determined by multiple unpaired t-tests. P values are shown as * if p<0.05, ** if p<0.01, * if p<0.001, **** if p<0.0001. FIG. 31B) Expanded WT PM21-NK cells and TIGIT KO PM21-NK cells (3333 NK cells/well) were co-cultured with A549 spheroids with or without non-Fc competent anti-TIGIT antibody or Fc-competent anti-TIGIT antibody (Tiragolumab) for 7 days. NK cell cytotoxicity was determined by kinetic live-cell imaging. Summary plot shows that WT PM21-NK cells show significantly lower cytotoxicity against A549 spheroids in the presence of Fc-competent anti-TIGIT antibody (solid red triangle) compared to non-Fc competent anti-TIGIT antibody (solid blue square), there was no significant change for TIGIT KO PM21-NK cells in the presence of non-Fc and Fc competent anti-TIGIT antibody (N=3 donors, each in triplicate). Data are presented as scatter plots or bar graphs with error bars representing standard deviation. Statistical significance was determined by multiple unpaired t-tests. P values are shown as * if p<0.05, ** if p<0.01, *** if p<0.001, **** if p<0.0001.

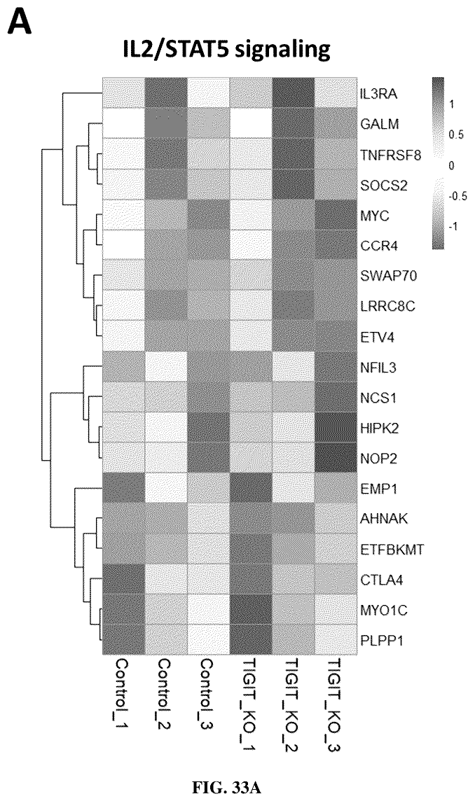

To test whether TIGIG KO NK cells exhibited improved in vivo persistence, 1×10⁶ of lung cancer cell line NCI-H358-GFP-luc or NCI-H1299-GFP-luc were injected to the intraperitoneal (i.p.) cavity of NSG (NOD-scid IL-2Rγnull) female mice and allowed to seed for 3 days. Mice were then treated with WT or TIGIT KO NK cells from one donor, injected i.p. along with IL-2 (25,000 U, 3×/week). Mice were euthanized 12 days after NK cell injection. Abdominal washes were analyzed by flow cytometry, gating on hCD45+ cells. The percentages of hNK (CD3—,CD56+) was determined in the hCD45+ population. More NK cells were recovered from mice treated with TIGIT KO NK cells (red triangles) compared to WT NK cells (black circles) from mice injected with either H358 or H1299 lung tumor cells, with H358-bearing mice reaching statistical significance (FIG. 32). Statistical significance was determined by unpaired t-test. P values are shown as * if p<0.05.

### REFERENCES

1. Shimasaki N, Jain A, Campana D. N K cells for cancer immunotherapy. Nat Rev Drug Discov. 2020; 1-19.
2. Xie G, Dong H, Liang Y, Ham J D, Rizwan R, Chen J. CAR-N K cells: A promising cellular immunotherapy for cancer. EBioMedicine. 2020; 59.
3. Liu E, Tong Y, Dotti G, Shaim H, Savoldo B, Mukherjee M, et al. Cord blood N K cells engineered to express IL-15 and a CD19-targeted CAR show long-term persistence and potent antitumor activity. Leukemia. 2018; 32(2):520-31.
4. Liu E, Marin D, Banerjee P, Macapinlac H A, Thompson P, Basar R, et al. Use of CAR-Transduced Natural Killer Cells in CD19-Positive Lymphoid Tumors. N Engl J Med. 2020; 382(6):545-53.
5. Vivier E, Tomasello E, Baratin M, Walzer T, Ugolini S. Functions of natural killer cells. Nat Immunol. 2008; 9(5):503-10.
6. Zhang Q, Bi J, Zheng X, Chen Y, Wang H, Wu W, et al. Blockade of the checkpoint receptor TIGIT prevents N K cell exhaustion and elicits potent anti-tumor immunity. Nat Immunol. 2018; 19(7):723-32.
7. Kamiya T, Seow S V, Wong D, Robinson M, Campana D. Blocking expression of inhibitory receptor NKG2A overcomes tumor resistance to N K cells. J Clin Invest. 2019; 129(5):2094-106.
8. Bi J, Tian Z. N K cell exhaustion. Front Immunol. 2017; 8(Jun.).
9. Da Silva I P, Gallois A, Jimenez-Baranda S, Khan S, Anderson A C, Kuchroo V K, et al. Reversal of N K-cell exhaustion in advanced melanoma by Tim-3 blockade. Cancer Immunol Res. 2014; 2(5):410-22.
10. Harjunpää H, Guillerey C. TIGIT as an emerging immune checkpoint. Clin Exp Immunol. 2020; 200(2):108-19.
11. Manieri N A, Chiang E Y, Grogan J L. TIGIT: A Key Inhibitor of the Cancer Immunity Cycle. Trends Immunol [Internet]. 2017 Jan. 1; 38(1):20-8. Available from: https://doi.org/10.1016/j.it.2016.10.002
12. Chauvin J M, Zarour H M. TIGIT in cancer immunotherapy. J Immunother Cancer. 2020; 8(2):1-7.
13. Zhang Q, Bi J, Zheng X, Chen Y, Wang H, Wu W, et al. Blockade of the checkpoint receptor TIGIT prevents N K cell exhaustion and elicits potent anti-tumor immunity. Nat Immunol [Internet]. 2018; 19(7):723-32.
14. Rodriguez-Abreu D, Johnson M L, Hussein M A, Cobo M, Patel A J, Secen N M, et al. Primary analysis of a randomized, double-blind, phase II study of the anti-TIGIT antibody tiragolumab (tira) plus atezolizumab (atezo) versus placebo plus atezo as first-line (1L) treatment in patients with P D-L1-selected NSCLC (CITYSCAPE). J Clin Oncol [Internet]. 2020 May 20; 38(15_suppl):9503. Available from: https://doi.org/10.1200/JCO.2020.38.15_suppl.9503
15. Yang F, Zhao L, Wei Z, Yang Y, Liu J, Li Y, et al. A Cross-Species Reactive TIGIT-Blocking Antibody Fc Dependently Confers Potent Antitumor Effects. J Immunol. 2020; 205(8):2156-68.
16. Preillon J, Cuende J, Rabolli V, Garnero L, Mercier M, Wald N, et al. Restoration of t-cell effector function, depletion of tregs, and direct killing of tumor cells: The multiple mechanisms of action of α-tigit antagonist antibodies. Mol Cancer Ther. 2021; 20(1):121-31.
17. Abel A M, Yang C, Thakar M S, Malarkannan S. Natural killer cells: Development, maturation, and clinical utilization. Front Immunol. 2018; 9(Aug.):1-23.
18. Daher M, Rezvani K. Outlook for new car-based therapies with a focus on car nk cells: What lies beyond car-engineered t cells in the race against cancer. Cancer Discov. 2021; 11(1):45-58.
19. Shimasaki N, Jain A, Campana D. N K cells for cancer immunotherapy. Nat Rev Drug Discov [Internet]. Available from: http://dx.doi.org/10.1038/s41573-019-0052-1
20. Lanier L L. Up on the tightrope: Natural killer cell activation and inhibition. Nat Immunol. 2008; 9(5):495-502.
21. Wang W, Erbe A K, Hank J A, Morris Z S, Sondel P M. N K cell-mediated antibody-dependent cellular cytotoxicity in cancer immunotherapy. Front Immunol. 2015; 6(Jul.).
22. Sanchez-Correa B, Valhondo I, Hassouneh F, Lopez-Sejas N, Pera A, Bergua J M, et al.

DNAM-1 and the TIGIT/PVRIG/TACTILE axis: Novel immune checkpoints for natural killer cell-based cancer immunotherapy. Vol. 11, Cancers. 2019. p. 1-15.
23. Selvan S R, Dowling J P. “adherent” versus Other Isolation Strategies for Expanding Purified, Potent, and Activated Human N K Cells for Cancer Immunotherapy. Biomed Res Int. 2015; 2015(September).
24. Oyer J L, Pandey V, Igarashi R Y, Somanchi S S, Zakari A, Solh M, et al. Natural killer cells stimulated with PM21 particles expand and biodistribute in vivo: Clinical implications for cancer treatment. Cytotherapy. 2016; 18(5):653-63.
25. Oyer J L, Gitto S B, Altomare D A, Copik A J. P D-L1 blockade enhances anti-tumor efficacy of N K cells. Oncoimmunology [Internet]. 2018; 7(11):1-11.
26. Han J H, Cai M, Grein J, Perera S, Wang H, Bigler M, et al. Effective Anti-tumor Response by TIGIT Blockade Associated With FcγR Engagement and Myeloid Cell Activation. Front Immunol. 2020; 11 (October):1-14.
27. Dolgin E. Antibody engineers seek optimal drug targeting TIGIT checkpoint. Nat Biotechnol. 2020; 38(9):1007-9.
28. Wu J, Mishra H K, Walcheck B. Role of ADAM17 as a regulatory checkpoint of CD16A in N K cells and as a potential target for cancer immunotherapy. J Leukoc Biol. 2019; 105(6):1297-303.
29. Bruhns P. Properties of mouse and human IgG receptors and their contribution to disease models. Blood. 2012; 119(24):5640-9.
30. Walcheck B, Wu J. iNK-CD64/16A cells: a promising approach for ADCC? Expert Opin Biol Ther [Internet]. 2019; 19(12):1229-32.

SEQUENCE

SEQ ID NO: 1 (polypeptide sequence for TIGIT)
MRWCLLLIWAQGLRQAPLASGMMTGTIETTGNISAEKGGSIILQCHLSSTTAQVTQVNW
EQQDQLLAICNADLGWHISPSFKDRVAPGPGLGLTLQSLTVNDTGEYFCIYHTYPDGTY
TGRIFLEVLESSVAEHGARFQIPLLGAMAATLVVICTAVIVVVALTRKKKALRIHSVEGD
LRRKSAGQEEWSPSAPSPPGSCVQAEAAPAGLCGEQRGEDCAELHDYFNVLSYRSLGN
CSFFTETG
SEQ ID NO: 2 (polypeptide sequence for PVRIG)
MRTEAQVPALQPPEPGLEGAMGHRTLVLPWVLLTLCVTAGTPEVWVQVRMEATELSSF
TIRCGFLGSGSISLVTVSWGGPNGAGGTTLAVLHPERGIRQWAPARQARWETQSSISLIL
EGSGASSPCANTTFCCKFASFPEGSWEACGSLPPSSDPGLSAPPTPAPILRADLAGILGVS
GVLLFGCVYLLHLLRRHKHRPAPRLQPSRTSPQAPRARAWAPSQASQAALHVPYATINT
SCRPATLDTAHPHGGPSWWASLPTHAAHRPQGPAAWASTPIPARGSFVSVENGLYAQA
GERPPHTGPGLTLFPDPRGPRAMEGPLGVR
SEQ ID NO: 3 (polypeptide sequence for CD96)
MEKKWKYCAVYYIIQIHFVKGVWEKTVNTEENVYATLGSDVNLTCQTQTVGFFVQMQ
WSKVTNKIDLIA VYHPQYGFYCAYGRPCESLVTFTETPENGSKWTLHLRNMSCSVSGR
YECMLVLYPEGIQTKIYNLLIQTHVTADEWNSNHTIEIEINQTLEIPCFQNSSSKISSEFTY
AWSVENSSTDSWVLLSKGIKEDNGTQETLISQNHLISNSTLLKDRVKLGTDYRLHLSPV
QIFDDGRKFSCHIRVGPNKILRSSTTVKVFAKPEIPVIVENNSTDVLVERRFTCLLKNVFP
KANITWFIDGSFLHDEKEGIYITNEERKGKDGFLELKSVLTRVHSNKPAQSDNLTIWCM
ALSPVPGNKVWNISSEKITFLLGSEISSTDPPLSVTESTLDTQPSPASSVSPARYPATSSVTL
VDVSALRPNTTPQPSNSSMTTRGFNYPWTSSGTDTKKSVSRIPSETYSSSPSGAGSTLHD
NVFTSTARAFSEVPTTANGSTKTNHVHITGIVVNKPKDGMSWPVIVAALLFCCMILFGL
GVRKWCQYQKEIMERPPPFKPPPPPIKYTCIQEPNESDLPYHEMETL
SEQ ID NO: 4 (polypeptide sequence for DNAM-1)
MDYPTLLLALLHVYRALCEEVLWHTSVPFAENMSLECVYPSMGILTQVEWFKIGTQQD
SIAIFSPTHGMVIRKPYAERVYFLNSTMASNNMTLFFRNASEDDVGYYSCSLYTYPQGT
WQKVIQVVQSDSFEAAVPSNSHIVSEPGKNVTLTCQPQMTWPVQAVRWEKIQPRQIDLL
TYCNLVHGRNFTSKFPRQIVSNCSHGRWSVIVIPDVTVSDSGLYRCYLQASAGENETFV
MRLTVAEGKTDNQYTLFVAGGTVLLLLFVISITTIIVIFLNRRRRRERRDLFTESWDTQK
APNNYRSPISTSQPTNQSMDDTREDIYVNYPTFSRRPKTRV
SEQ ID NO: 5 (polypeptide sequence for LAG3)
MWEAQFLGLLFLQPLWVAPVKPLQPGAEVPVVWAQEGAPAQLPCSPTIPLQDLSLLRR
AGVTWQHQPDSGPPAAAPGHPLAPGPHPAAPSSWGPRPRRYTVLSVGPGGLRSGRLPL
QPRVQLDERGRQRGDFSLWLRPARRADAGEYRAAVHLRDRALSCRLRLRLGQASMTA
SPPGSLRASDWVILNCSFSRPDRPASVHWFRNRGQGRVPVRESPHHHLAESFLFLPQVSP
MDSGPWGCILTYRDGFNVSIMYNLTVLGLEPPTPLTVYAGAGSRVGLPCRLPAGVGTRS
FLTAKWTPPGGGPDLLVTGDNGDFTLRLEDVSQAQAGTYTCHIHLQEQQLNATVTLAII
TVTPKSFGSPGSLGKLLCEVTPVSGQERFVWSSLDTPSQRSFSGPWLEAQEAQLLSQPW
QCQLYQGERLLGAAVYFTELSSPGAQRSGRAPGALPAGHLLLFLILGVLSLLLLVTGAF
GFHLWRRQWRPRRFSALEQGIHPPQAQSKIEELEQEPEPEPEPEPEPEPEPEPEQ
SEQ ID NO: 6 (polypeptide sequence for PD-1)
MQIPQAPWPVVWAVLQLGWRPGWFLDSPDRPWNPPTFSPALLVVTEGDNATFTCSFSN
TSESFVLNWYRMSPSNQTDKLAAFPEDRSQPGQDCRFRVTQLPNGRDFHMSVVRARRN
DSGTYLCGAISLAPKAQIKESLRAELRVTERRAEVPTAHPSPSPRPAGQFQTLVVGVVGG
LLGSLVLLVWVLAVICSRAARGTIGARRTGQPLKEDPSAVPVFSVDYGELDFQWREKTP
EPPVPCVPEQTEYATIVFPSGMGTSSPARRGSADGPRSAQPLRPEDGHCSWPL
SEQ ID NO: 7 (polypeptide sequence for CTLA-4)
MACLGFQRHKAQLNLATRTWPCTLLFFLLFIPVFCKAMHVAQPAVVLASSRGIASFVCE
YASPGKATEVRVTVLRQADSQVTEVCAATYMMGNELTFLDDSICTGTSSGNQVNLTIQ
GLRAMDTGLYICKVELMYPPPYYLGIGNGTQIYVIDPEPCPDSDFLLWILAAVSSGLFFY
SFLLTAVSLSKMLKKRSPLTTGVYVKMPPTEPECEKQFQPYFIPIN
SEQ ID NO: 8 (polypeptide sequence for TIM-3)
MFSHLPFDCVLLLLLLLLTRSSEVEYRAEVGQNAYLPCFYTPAAPGNLVPVCWGKGACP
VFECGNVVLRTDERDVNYWTSRYWLNGDFRKGDVSLTIENVTLADSGIYCCRIQIPGIM
NDEKFNLKLVIKPAKVTPAPTRQRDFTAAFPRMLTTRGHGPAETQTLGSLPDINLTQIST
LANELRDSRLANDLRDSGATIRIGIYIGAGICAGLALALIFGALIFKWYSHSKEKIQNLSLI
SLANLPPSGLANAVAEGIRSEENIYTIEENVYEVEEPNEYYCYVSSRQQPSQPLGCRFAM
P
SEQ ID NO: 9 (polypeptide sequence for NKG2A)
MDNQGVIYSDLNLPPNPKRQQRKPKGNKNSILATEQEITYAELNLQKASQDFQGNDKT
YHCKDLPSAPEKLIVGILGIICLILMASVVTIVVIPSTLIQRHNNSSLNTRTQKARHCGHCP
EEWITYSNSCYYIGKERRTWEESLLACTSKNSSLLSIDNEEEMKFLSIISPSSWIGVFRNSS
HHPWVTMNGLAFKHEIKDSDNAELNCAVLQVNRLKSAQCGSSIIYHCKHKL
SEQ ID NO: 10 (polynucleotide sequence for TIGIT, RefSeq NM_173799)
ACATCTGCTTCCTGTAGGCCCTCTGGGCAGAAGCATGCGCTGGTGTCTCCTCCTGATC
TGGGCCCAGGGGCTGAGGCAGGCTCCCCTCGCCTCAGGAATGATGACAGGCACAATA
GAAACAACGGGGAACATTTCTGCAGAGAAAGGTGGCTCTATCATCTTACAATGTCAC
CTCTCCTCCACCACGGCACAAGTGACCCAGGTCAACTGGGAGCAGCAGGACCAGCT
TCTGGCCATTTGTAATGCTGACTTGGGGTGGCACATCTCCCCATCCTTCAAGGATCGA
GTGGCCCCAGGTCCCGGCCTGGGCCTCACCCTCCAGTCGCTGACCGTGAACGATACA
GGGGAGTACTTCTGCATCTATCACACCTACCCTGATGGGACGTACACTGGGAGAATCT
TCCTGGAGGTCCTAGAAAGCTCAGTGGCTGAGCACGGTGCCAGGTTCCAGATTCCAT
TGCTTGGAGCCATGGCCGCGACGCTGGTGGTCATCTGCACAGCAGTCATCGTGGTGG
TCGCGTTGACTAGAAAGAAGAAAGCCCTCAGAATCCATTCTGTGGAAGGTGACCTCA
GGAGAAAATCAGCTGGACAGGAGGAATGGAGCCCCAGTGCTCCCTCACCCCCAGGA
AGCTGTGTCCAGGCAGAAGCTGCACCTGCTGGGCTCTGTGGAGAGCAGCGGGGAGA
GGACTGTGCCGAGCTGCATGACTACTTCAATGTCCTGAGTTACAGAAGCCTGGGTAA
CTGCAGCTTCTTCACAGAGACTGGTTAGCAACCAGAGGCATCTTCTGGAAGATACAC
TTTTGTCTTTGCTATTATAGATGAATATATAAGCAGCTGTACTCTCCATCAGTGCTGCGT
GTGTGTGTGTGTGTGTATGTGTGTGTGTGTTCAGTTGAGTGAATAAATGTCATCCTCTT
CTCCATCTTCATTTCCTTGGCCTTTTCGTTCTATTCCATTTTGCATTATGGCAGGCCTAG
GGTGAGTAACGTGGATCTTGATCATAAATGCAAAATTAAAAAATATCTTGACCTGGTT
TTAAATCTGGCAGTTTGAGCAGATCCTATGTCTCTGAGAGACACATTCCTCATAATGG
CCAGCATTTTGGGCTACAAGGTTTTGTGGTTGATGATGAGGATGGCATGACTGCAGAG
CCATCCTCATCTCATTTTTTCACGTCATTTTCAGTAACTTTCACTCATTCAAAGGCAGG
TTATAAGTAAGTCCTGGTAGCAGCCTCTATGGGGAGATTTGAGAGTGACTAAATCTTG
GTATCTGCCCTCAAGAACTTACAGTTAAATGGGGAGACAATGTTGTCATGAAAAGGT
ATTATAGTAAGGAGAGAAGGAGACATACACAGGCCTTCAGGAAGAGACGACAGTTT
GGGGTGAGGTAGTTGGCATAGGCTTATCTGTGATGAAGTGGCCTGGGAGCACCAAGG
GGATGTTGAGGCTAGTCTGGGAGGAGCAGGAGTTTTGTCTAGGGAACTTGTAGGAAA
TTCTTGGAGCTGAAAGTCCCACAAAGAAGGCCCTGGCACCAAGGGAGTCAGCAAAC
TTCAGATTTTATTCTCTGGGCAGGCATTTCAAGTTTCCTTTTGCTGTGACATACTCATC
CATTAGACAGCCTGATACAGGCCTGTAGCCTCTTCCGGCCGTGTGTGCTGGGGAAGC
CCCAGGAAACGCACATGCCCACACAGGGAGCCAAGTCGTAGCATTTGGGCCTTGATC
TACCTTTTCTGCATCAATACACTCTTGAGCCTTTGAAAAAAGAACGTTTCCCACTAAA
AAGAAAATGTGGATTTTTAAAATAGGGACTCTTCCTAGGGGAAAAAGGGGGGCTGGG
AGTGATAGAGGGTTTAAAAAATAAACACCTTCAAACTAACTTCTTCGAACCCTTTTAT
TCACTCCCTGACGACTTTGTGCTGGGGTTGGGGTAACTGAACCGCTTATTTCTGTTTA
ATTGCATTCAGGCTGGATCTTAGAAGACTTTTATCCTTCCACCATCTCTCTCAGAGGA
ATGAGCGGGGAGGTTGGATTTACTGGTGACTGATTTTCTTTCATGGGCCAAGGAACTG
AAAGAGAATGTG
SEQ ID NO: 11 (sequence for gRNA that targets TIGIT)
ACCCTGATGGGACGTACACT

What is claimed is:

1. An engineered NK cell which is suppressed in the expression of T Cell Immunoreceptor with Ig And ITIM Domains (TIGIT).

2. The engineered NK cell of claim 1, wherein the expression of TIGIT is suppressed by a deletion of a TIGIT gene or a fragment thereof.

3. The engineered NK cell of claim 1 or 2, wherein the expression of TIGIT is suppressed using a method comprising introducing into the NK cell a CRISPR/Cas endonuclease (Cas)9 system with a CRISPR/Cas guide RNA, wherein the guide RNA targets the TIGIT gene or a fragment thereof.

4. The engineered NK cell of claim 1, wherein the expression of TIGIT is suppressed by a siRNA or a shRNA that targets a TIGIT polynucleotide.

5. The engineered NK cell of any one of claims 1-4, wherein the engineered NK cell is suppressed in the expression of an inhibitory receptor selected from the group consisting of poliovirus receptor-related immunoglobulin domain-containing (PVRIG), CD96, lymphocyte activating 3 (LAG3), TIM-3, NKG2A, PD-1, and CTLA-4.

6. The engineered NK cell of any one of claims 1-5, wherein the NK cell is a primary NK cell or a NK cell line.

7. The engineered NK cell of any one of claims 1-6, wherein the NK cell is an expanded NK cell or a non-expanded NK cell.

8. The engineered NK cell of claim 7, wherein the expanded NK cell is exposed in vitro or ex vivo to an NK cell expanding composition.

9. The engineered NK cell of claim 8, wherein the NK cell expanding composition comprises a feeder cell, an engineered PM particle, or an exosome.

10. The engineered NK cell of claim 9, wherein the feeder cell or engineered particle comprises an Fc domain bound to an external surface thereof.

11. The engineered NK cell of any one of claims 8-10, wherein the NK cell expanding composition further comprises an NK cell effector agent.

12. The engineered NK cell of claim 11, wherein the NK cell effector agent comprises IL-21 and/or 41BBL.

13. A method of treating, decreasing, inhibiting, reducing, ameliorating, and/or preventing a cancer, metastasis, or an infectious disease in a subject comprising administering to the subject a therapeutically effective amount of the engineered NK cell of any one of claim 1-12.

14. The method of claim 13, further comprising administering to the subject a therapeutically effective amount of a TIGIT inhibitor.

15. The method of claim 14, wherein the TIGIT inhibitor is an anti-TIGIT antibody.

16. The method of claim 15, wherein the anti-TIGIT antibody comprises a fragment crystallizable region (Fc region) that binds to an Fc receptor.

17. The method of claim 15, wherein the anti-TIGIT antibody lacks a Fc region or comprises a Fc region having a reduced affinity to an Fc receptor relative to a reference control.

18. The method of any one of claims 13-17, further comprising administering to subject a therapeutically effective amount of a checkpoint blockade.

19. The method of claim 18, wherein the checkpoint blockade comprises a PD-1 inhibitor, a PD-L1 inhibitor, a PD-L2 inhibitor, or a CTLA-4 inhibitor.

20. A method of treating, decreasing, inhibiting, reducing, ameliorating, and/or preventing a cancer, metastasis, or an infectious disease in a subject comprising administering to the subject a therapeutically effective amount of an engineered NK cell, wherein the engineered NK cell is suppressed in the expression of T Cell Immunoreceptor with Ig And ITIM Domains (TIGIT).

21. The method of claim 20, wherein the expression of TIGIT is suppressed by a deletion of a TIGIT gene or a fragment thereof.

22. The method of claim 20 or 21, wherein the expression of TIGIT is suppressed using a method comprising introducing into the NK cell a CRISPR/Cas endonuclease (Cas)9 system with a CRISPR/Cas guide RNA, wherein the guide RNA targets the TIGIT gene or a fragment thereof.

23. The method of claim 22, wherein the expression of TIGIT is suppressed by a siRNA or a shRNA that targets a TIGIT polynucleotide.

24. The method of any one of claims 20-23, wherein the engineered NK cell is suppressed in the expression of an inhibitory receptor selected from the group consisting of poliovirus receptor-related immunoglobulin domain-containing (PVRIG), CD96, lymphocyte activating 3 (LAG3), TIM-3, NKG2A, PD-1, and CTLA-4.

25. The method of any one of claims 20-24, wherein the NK cell is a primary NK cell or a NK cell line.

26. The method of any one of claims 20-25, wherein the NK cell is an expanded NK cell or a non-expanded NK cell.

27. The method of claim 26, wherein the expanded NK cell is exposed in vitro or ex vivo to an NK cell expanding composition.

28. The method of claim 27, wherein the NK cell expanding composition comprises a feeder cell, an engineered PM particle, or an exosome.

29. The method of claim 28, wherein the feeder cell or engineered particle comprises an Fc domain bound to an external surface thereof.

30. The method of any one of claims 27-29, wherein the NK cell expanding composition further comprises an NK cell effector agent.

31. The method of claim 30, wherein the NK cell effector agent comprises IL-21 and/or 41BBL.

32. The method of any one of claims 20-31, further comprising administering to the subject a therapeutically effective amount of a TIGIT inhibitor.

33. The method of claim 32, wherein the TIGIT inhibitor is an anti-TIGIT antibody.

34. The method of claim 33, wherein the anti-TIGIT antibody comprises a fragment crystallizable region (Fc region) that binds to an Fc receptor.

35. The method of claim 34, wherein the anti-TIGIT antibody lacks a Fc region or comprises a Fc region having a reduced affinity to an Fc receptor relative to a reference control.

36. The method of any one of claims 20-35, further comprising administering to subject a therapeutically effective amount of a checkpoint blockade.

37. The method of claim 36, wherein the checkpoint blockade comprises a PD-1 inhibitor, a PD-L1 inhibitor, a PD-L2 inhibitor, or a CTLA-4 inhibitor.

38. An in vitro or ex vivo method for reactivating an NK cell, reversing NK cell exhaustion, and/or enhancing NK cell function, the method comprising suppressing the expression of T Cell Immunoreceptor with Ig And ITIM Domains (TIGIT) of the NK cell or incubating the NK cell with a TIGIT inhibitor.

39. The method of claim 38, wherein the expression of TIGIT is suppressed by a deletion of a TIGIT gene or a fragment thereof.

40. The method of claim 38 or 39, wherein the expression of TIGIT is suppressed using a method comprising introducing into the NK cell a CRISPR/Cas endonuclease (Cas)9 system with a CRISPR/Cas guide RNA, wherein the guide RNA targets the TIGIT gene or a fragment thereof.

41. The method of claim 40, wherein the expression of TIGIT is suppressed by a siRNA or a shRNA that targets a TIGIT polynucleotide.

42. The method of any one of claims 38-41, wherein the engineered NK cell is suppressed in the expression of an inhibitory receptor selected from the group consisting of poliovirus receptor-related immunoglobulin domain-containing (PVRIG), CD96, lymphocyte activating 3 (LAG3), TIM-3, NKG2A, PD-1, and CTLA-4.

43. The method of any one of claims 38-42, wherein the NK cell is a primary NK cell or a NK cell line.

44. The method of any one of claims 38-42, wherein the NK cell is an expanded NK cell or a non-expanded NK cell.

45. The method of claim 44, wherein the expanded NK cell is exposed in vitro to an NK cell expanding composition.

46. The method of claim 45, wherein the NK cell expanding composition comprises a feeder cell, an engineered PM particle, or an exosome.

47. The method of claim 46, wherein the feeder cell or engineered particle comprises an Fc domain bound to an external surface thereof.

48. The method of any one of claims 45-47, wherein the NK cell expanding composition further comprises an NK cell effector agent.

49. The method of claim 48, wherein the NK cell effector agent comprises IL-21 and/or 41BBL.

50. The method of any one of claims 38-49, further comprising incubating the NK cell with a TIGIT inhibitor.

51. The method of claim 38 or 50, wherein the TIGIT inhibitor is an anti-TIGIT antibody.

52. The method of claim 51, wherein the anti-TIGIT antibody comprises a fragment crystallizable region (Fc region) that binds to an Fc receptor.

53. The method of claim 51, wherein the anti-TIGIT antibody lacks a Fc region or comprises a Fc region having a reduced affinity to an Fc receptor relative to a reference control.

54. The method of any one of claims 38-53, further comprising incubating the NK cell with a checkpoint blockade.

55. The method of claim 54, wherein the checkpoint blockade comprises a PD-1 inhibitor, a PD-L1 inhibitor, a PD-L2 inhibitor, or a CTLA-4 inhibitor.

56. A method of treating, decreasing, inhibiting, reducing, ameliorating, and/or preventing a cancer, metastasis, or an infectious disease in a subject comprising administering to the subject a therapeutically effective amount of an NK cell prepared by the method of any one of claims 38-55.

57. A method of treating, decreasing, inhibiting, reducing, ameliorating, and/or preventing a cancer, metastasis, or an infectious disease in a subject comprising administering to the subject a therapeutically effective amount of an NK cell and a therapeutically effective amount of a T Cell Immunoreceptor with Ig And ITIM Domains (TIGIT) inhibitor.

58. The method of claim 57, wherein the NK cell is a primary NK cell or a NK cell line.

59. The method of claim 57 or 58, wherein the NK cell is an expanded NK cell.

60. The method of claim 59, wherein the expanded NK cell is exposed in vitro or ex vivo to an NK cell expanding composition.

61. The method of claim 60, wherein the NK cell expanding composition comprises a feeder cell, an engineered PM particle, or an exosome.

62. The method of claim 61, wherein the feeder cell or engineered particle comprises an Fc domain bound to an external surface thereof.

63. The method of any one of claims 60-62, wherein the NK cell expanding composition further comprises an NK cell effector agent.

64. The method of claim 63, wherein the NK cell effector agent comprises IL-21 and/or 41BBL.

65. The method of any one of claims 57-64, wherein the TIGIT inhibitor is an anti-TIGIT antibody.

66. The method of claim 65, wherein the anti-TIGIT antibody comprises a fragment crystallizable region (Fc region) that binds to an Fc receptor.

67. The method of claim 65, wherein the anti-TIGIT antibody lacks a Fc region or comprises a Fc region having a reduced affinity to an Fc receptor relative to a reference control.

68. The method of any one of claims 57-67, further comprising administering to subject a therapeutically effective amount of a checkpoint blockade.

69. The method of claim 68, wherein the checkpoint blockade comprises a PD-1 inhibitor, a PD-L1 inhibitor, a PD-L2 inhibitor, or a CTLA-4 inhibitor.

70. A method of treating, decreasing, inhibiting, reducing, ameliorating, and/or preventing a cancer, metastasis, or an infectious disease in a subject comprising administering to the subject a therapeutically effective amount of an engineered NK cell, wherein the engineered NK cell is suppressed in the expression of T Cell Immunoreceptor with Ig And ITIM Domains (TIGIT) and wherein the engineered NK cell is incubated in vitro or ex vivo with a TIGIT inhibitor prior to the administering step.

71. The method of claim 70, wherein the expression of TIGIT is suppressed by a deletion of a TIGIT gene or a fragment thereof.

72. The method of claim 70 or 71, wherein the expression of TIGIT is suppressed using a method comprising introducing into the NK cell a CRISPR/Cas endonuclease (Cas)9 system with a CRISPR/Cas guide RNA, wherein the guide RNA targets the TIGIT gene or a fragment thereof.

73. The method of claim 72, wherein the expression of TIGIT is suppressed by a siRNA or a shRNA that targets a TIGIT polynucleotide.

74. The method of any one of claims 70-73, wherein the engineered NK cell is suppressed in the expression of an inhibitory receptor selected from the group consisting of poliovirus receptor-related immunoglobulin domain-containing (PVRIG), CD96, lymphocyte activating 3 (LAG3), TIM-3, NKG2A, PD-1, and CTLA-4.

75. The method of any one of claims 70-74, wherein the NK cell is a primary NK cell or a NK cell line.

76. The method of any one of claims 70-75, wherein the NK cell is an expanded NK cell or a non-expanded NK cell.

77. The method of claim 76, wherein the expanded NK cell is exposed in vitro or ex vivo to an NK cell expanding composition.

78. The method of claim 77, wherein the NK cell expanding composition comprises a feeder cell, an engineered PM particle, or an exosome.

79. The method of claim 78, wherein the feeder cell or engineered particle comprises an Fc domain bound to an external surface thereof.

80. The method of any one of claims 77-79, wherein the NK cell expanding composition further comprises an NK cell effector agent.

81. The method of claim 80, wherein the NK cell effector agent comprises IL-21 and/or 41BBL.

82. The method of any one of claims 70-81, further comprising administering to the subject a therapeutically effective amount of a TIGIT inhibitor.

83. The method of any one of claims 70-82, wherein the TIGIT inhibitor is an anti-TIGIT antibody.

84. The method of claim 83, wherein the anti-TIGIT antibody comprises a fragment crystallizable region (Fc region) that binds to an Fc receptor.

85. The method of claim 84, wherein the anti-TIGIT antibody lacks a Fc region or comprises a Fc region having a reduced affinity to an Fc receptor relative to a reference control.

86. The method of any one of claims 70-85, further comprising administering to subject a therapeutically effective amount of a checkpoint blockade.

87. The method of claim 86, wherein the checkpoint blockade comprises a PD-1 inhibitor, a PD-L1 inhibitor, a PD-L2 inhibitor, or a CTLA-4 inhibitor.


SEQUENCE LISTING (11 sequences)

SEQ ID NO: 1 - protein, 244 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 2 - protein, 326 aa, synthetic construct
MRTEAQVPALQPPEPGLEGAMGHRTLVLPWVLLTLCVTAGTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLAGILGVSGVLLFGCVYLLHLLRRHKHRPAPRLQPSRTSPQAPRARAWAPSQASQAALHVPYATINTSCRPATLDTAHPHGGPSWWASLPTHAAHRPQGPAAWASTPIPARGSFVSVENGLYAQAGERPPHTGPGLTLFPDPRGPRAMEGPLGVR

SEQ ID NO: 3 - protein, 585 aa, synthetic construct
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 4 - protein, 336 aa, synthetic construct
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 5 - protein, 524 aa, synthetic construct
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQ

SEQ ID NO: 6 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 7 - protein, 223 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 8 - protein, 301 aa, synthetic construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 9 - protein, 233 aa, synthetic construct
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 10 - DNA, 2083 bp, synthetic construct
  source 1..2083: mol_type = other DNA
acatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgtactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaaccgcttatttctgtttaattgcattcaggctggatcttagaagacttttatccttccaccatctctctcagaggaatgagcggggaggttggatttactggtgactgattttctttcatgggccaaggaactgaaagagaatgtg

SEQ ID NO: 11 - DNA, 20 bp, synthetic construct
  source 1..20: mol_type = other DNA
accctgatgggacgtacact